(19) United States
(12) Patent Application Publication
(10) Pub. No.: US 2024/0192225 A1
(43) Pub. Date: Jun. 13, 2024

(54) METHOD FOR EPITOPE BINNING OF NOVEL MONOCLONAL ANTIBODIES

(71) Applicant: 10x Genomics, Inc., Pleasanton, CA (US)

(72) Inventors: Wyatt James MCDONNELL, Pleasanton, CA (US); Michael John Terry STUBBINGTON, Counterslip Bristol (GB); Ravi RAMENANI, Pleasanton, CA (US)

(21) Appl. No.: 18/453,735

(22) Filed: Aug. 22, 2023

Related U.S. Application Data
(63) Continuation of application No. PCT/US2022/017329, filed on Feb. 22, 2022.
(60) Provisional application No. 63/152,558, filed on Feb. 23, 2021.
(60) Provisional application No. 63/152,571, filed on Feb. 23, 2021.

(51) Int. Cl. G01N 33/68; C12N 15/10

(52) U.S. Cl. CPC G01N 33/6878; C12N 15/1065; G01N 33/6854

(57) ABSTRACT

The present disclosure generally relates to methods and systems for obtaining epitope specify of candidate antibodies against one or more known antibodies. Further, the present disclosure also provides systems and methods for epitope binning.

### CROSS-REFERENCE TO RELATED APPLICATIONS

This application claims the benefit of U.S. Provisional Patent Application Nos. 63/152,558, filed Feb. 23, 2021, and 63/152,571, filed Feb. 23, 2021, which are incorporated herein by reference in their entireties.

### FIELD OF DISCLOSURE

The present disclosure generally relates to methods and systems for obtaining epitope specify of candidate antibodies against one or more known antibodies. Further, the present disclosure also provides systems and methods for epitope binning.

### BACKGROUND

In developing therapeutic monoclonal antibodies (mAbs), the selection of appropriate affinity, specificity, and biophysical properties is essential. A mAb's epitope generally correlates with its functional activity. Early-stage therapeutic antibody discovery efforts often generate large panels of mAbs per target. Therefore, it is helpful to organize mAbs into epitope families or “bins.” Epitope binning is used to characterize the binding of mAbs to an antigen. An epitope is a part of an antigenic or immunogenic determinant in a molecule such as an antigen, e.g., a part in or fragment of the molecule that is recognized by the immune system. A single antigen can have multiple epitopes, and specific antibodies can have specificity and affinity for binding to a particular epitope. In epitope binning, mAbs specific to the same target protein are typically tested pairwise against all mAbs in a set to assess whether they block one another's binding to a specific site of the antigen or not. Monoclonal antibodies that target similar epitopes often share a similar function. Thus, identifying an epitope bin with functional activity can significantly narrow down potential leads. With the high cost of developing a therapeutic mAb, the ability to identify a few high quality leads with relevant epitopes early in the discovery process is tremendously advantageous. Epitope binning usually takes significant time, sample volume, and manpower for experimental setup and data analysis. This has limited its application to small numbers of samples. Further, mAbs in different epitope bin can be combined as therapeutic cocktails.

The present disclosure provides methods and systems to rapidly identify a region of interest and set(s) of epitopes targeted by any antibody-based therapy, including monoclonal antibody, multi-specific antibody-based methods, and chimeric antigen receptors, etc. This enables pharmaceutical companies to screen their existing candidates potentially in a fraction of the time required to screen these antibodies via existing methods, while returning the same information at much higher throughput.

### SUMMARY

Provided herein, among others, is a method comprising: (a) obtaining a first epitope specificity of a first plurality of cells and a second epitope specificity of a second plurality of cells by contacting an antigen with the first plurality of cells and the second plurality of cells, wherein the antigen is labeled with a reporter oligonucleotide comprising a reporter sequence, and wherein the first plurality of cells is engineered to express a known antibody and the second plurality of cells is engineered to express a candidate antibody; (b) generating, i) a first reduced dimension representation of the first epitope specificity of the first plurality of cells expressing the known antibody; and ii) a second reduced dimension representation of the second epitope specificity of the second plurality of cells expressing the candidate antibody; (c) determining, based at least on the first reduced dimension representation and the second reduced dimension representation, whether the second plurality of cells exhibit a novel epitope specificity or an epitope specificity associated with the first plurality of cells.

In another embodiment, a method is provided. The method comprises (a) obtaining a first epitope specificity of a first plurality of cells and a second epitope specificity of a second plurality of cells by contacting an antigen with the first plurality of cells and the second plurality of cells, wherein the antigen is labeled with a reporter oligonucleotide comprising a reporter sequence, and wherein the first plurality of cells is engineered to express a known antibody and the second plurality of cells is engineered to express a candidate antibody; and (b) applying, by at least one data processor, one or more data analysis techniques to determine whether the second plurality of cells exhibit a novel epitope specificity or an epitope specificity associated with the first plurality of cells.

In some embodiments, the obtaining the first epitope specificity and the second epitope specificity further comprises: (i) generating a plurality of single cell suspensions, wherein each of the plurality of single cell suspensions comprises one of either the first plurality of cells or the second plurality of cells bound to or not bound to the antigen; (ii) generating a library of barcoded nucleic acid molecules from the first plurality of cells and the second plurality of cells, wherein the library of barcoded nucleic acid molecules comprises the reporter sequence or complement thereof and sequences corresponding to immune receptors; and (iii) sequencing the library of barcoded nucleic acid molecules.

In some embodiments, the obtaining the first epitope specificity and the second epitope specificity further comprises: (i) generating a plurality of single cell suspensions, wherein each of the plurality of single cell suspensions comprises one of either the first plurality of cells or the second plurality of cells bound to or not bound to the antigen; (ii) generating a library of barcoded nucleic acid molecules from the first plurality of cells and the second plurality of cells, wherein the library of barcoded nucleic acid molecules comprises the reporter sequence or complement thereof and sequences corresponding to immune receptors; and (iii) sequencing the library of barcoded nucleic acid molecules.

In some embodiments, (i) comprises: following the contacting the antigen with the first plurality of cells and the second plurality of cells, partitioning the first plurality of cells and the second plurality of cells into a plurality of partitions, wherein a partition of the plurality of partitions comprises a cell of the first plurality of cells or the second plurality of cells bound to the antigen, and a plurality of nucleic acid barcode molecules wherein a first nucleic acid barcode molecule of the plurality of nucleic acid barcode molecules comprises a partition barcode sequence.

In some embodiments, (ii) generating a library of barcoded nucleic acid molecules from the first plurality of cells and the second plurality of cells comprises a. in the partition, coupling the first nucleic acid barcode molecule to the reporter oligonucleotide, and b. using the reporter oligonucleotide coupled to the first nucleic acid barcode molecule to generate a first barcoded nucleic acid molecule comprising the reporter sequence or a reverse complement thereof and the partition barcode sequence or a reverse complement thereof.

In some embodiments, a second nucleic acid barcode molecule of the plurality of nucleic acid barcode molecules comprises the partition barcode sequence, and (ii) generating a library of barcoded nucleic acid molecules from the first plurality of cells and the second plurality of cells further comprises a. in the partition, coupling the second nucleic acid barcode molecule to a nucleic acid analyte of the cell bound to the antigen, the nucleic acid analyte comprising a sequence of an immune receptor expressed by the cell bound to the antigen, or a reverse complement thereof, and b. using the nucleic acid analyte of the cell bound to the antigen coupled to the second nucleic acid barcode molecule to generate a second barcoded nucleic acid molecule comprising the sequence of the immune receptor expressed by the cell bound to the antigen, or a reverse complement thereof.

In some embodiments, the plurality of nucleic acid barcode molecules is attached to a bead, and the partition barcode sequence identifies the bead. In some embodiments, the first nucleic acid barcode molecule comprises a first capture sequence configured to couple to the reporter oligonucleotide. In some embodiments, the reporter oligonucleotide further comprises a capture handle sequence complementary to the first capture sequence. In some embodiments, the second nucleic acid barcode molecule further comprises a second capture sequence configured to couple to the nucleic acid analyte of the cell bound to the antigen. In some embodiments, the nucleic acid analyte is an mRNA analyte or a cDNA molecule generated from the mRNA analyte. In some embodiments, the second capture sequence is a template switch oligonucleotide sequence. In some embodiments, the first capture sequence and the second capture sequence are identical. In some embodiments, the first capture sequence and the second capture sequence are different. In some embodiments, the partition is a droplet. In some embodiments, the partition is a well.

In some embodiments, the method further comprises contacting the first plurality of cells with a first cell group labeling agent comprising a first cell group reporter oligonucleotide, the first cell group reporter oligonucleotide comprising a first cell group reporter sequence that identifies the first plurality of cells, and contacting the second plurality of cells with a second cell group labeling agent, the second cell group labeling agent comprising a second cell group reporter oligonucleotide comprising a second cell group reporter sequence that identifies the second plurality of cells.

In some embodiments, a third nucleic acid barcode molecule of the plurality of nucleic acid barcode molecules comprises the partition barcode sequence, and (ii) generating a library of barcoded nucleic acid molecules from the first plurality of cells and the second plurality of cells comprises a. in the partition, coupling the third nucleic acid barcode molecule to the first cell group reporter oligonucleotide or to the second cell group reporter oligonucleotide, and b. using the third nucleic acid barcode molecule coupled to the first cell group reporter oligonucleotide or to the second cell group reporter oligonucleotide to generate a third barcoded nucleic acid molecule comprising the first cell group reporter sequence or the second cell group reporter sequence, or a reverse complement thereof, and the partition barcode sequence or a reverse complement thereof.

In some embodiments, the method further comprises determining a sequence of the first barcoded nucleic acid molecule or a derivative thereof, the second barcoded nucleic acid molecule or a derivative thereof, and/or the third barcoded nucleic acid molecule or a derivative thereof.

In some embodiments, the first reduced dimension representation is generated by decomposing a first matrix including a first dataset indicating the first epitope specificity of the first plurality of cells, and wherein the second reduced dimension representation is generated by decomposing a second matrix including a second dataset indicating the second epitope specificity of the second plurality of cells. In some embodiments, the first matrix and/or the second matrix are decomposed by applying a principle component analysis (PCA), a neighborhood component analysis, a linear discriminant analysis, and/or a non-negative matrix factorization.

In some embodiments, each of the first matrix and the second matrix includes a row corresponding to the antigen, wherein each column in the first matrix corresponds to one of the first plurality of cells expressing the known antibody, and wherein each column in the second matrix corresponds to one of the second plurality of cells expressing the candidate antibody.

In some embodiments, each element in the first matrix comprises a count of each of the first plurality of cells bound to the antigen, and wherein each element in the second matrix comprises a count of each of the second plurality of cells bound to the antigen.

In some embodiments, the first reduced dimension representation and the second reduced dimension representation are generated by embedding a reduced dimensional space.

In some embodiments, the reduced dimensional space is embedded by applying a t-distributed stochastic neighbor embedding (t-SNE), a uniform manifold approximation and projection (UMAP), and/or a generalized linear model principle component analysis (GLMPCA).

In some embodiments, the first reduced dimension representation and the second reduced dimension representation are generated by applying a graph-based embedding and reduction technique.

In some embodiments, the reporter oligonucleotide comprises an additional functional sequence. In certain embodiments, the additional functional sequence is selected from at least one functional sequence, at least one common barcode, at least one capture sequence, and at least one UMI.

In some embodiments, the antigen is selected from the group consisting of a protein, a viral-like particle, and a nanoparticle. In certain embodiments, the antigen is a protein. In some embodiments, the antigen comprises a point mutation. In some embodiments, the point mutation alters the epitope specificity of the known antibody.

In some embodiments, the first reduced dimension representation includes a first cluster of cells corresponding to the first plurality of cells, and wherein the second reduced dimension representation includes a second cluster of cells corresponding to the second plurality of cells.

In some embodiments, whether the second plurality of cells exhibit the novel epitope specificity or the epitope specificity associated with the first plurality of cells is determined based at least on a distribution of the second cluster of cells relative to the first cluster of cells.

In some embodiments, the method provided herein further comprises applying a clustering algorithm to validate the distribution of the second cluster of cells relative to the first cluster of cells.

In some embodiments, the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

In some embodiments, the second plurality of cells are determined to exhibit the epitope specificity of the first plurality of cells based at least on the second cluster of cells being within a threshold distance of the first cluster of cells.

In some embodiments, the second plurality of cells are determined to exhibit the novel epitope specificity based at least on the second cluster of cells being more than a threshold distance away from the first cluster of cells.

In some embodiments, at least one of the first dataset and the second dataset include one or more negative control antigens.

In some embodiments, the method provided herein further comprises (i) determining, for the first plurality of cells expressing the known antibody, a first plurality of scores representative of an epitope binding property of each of the first plurality of cells; (ii) determining, for the second plurality of cells expressing the candidate antibody, a second plurality of scores representative of the epitope binding property of each of the second plurality of cells; (iii) generating a first distribution of the first plurality of scores and a second distribution of the second plurality of scores; and (iv) determining, based at least on a comparison between the first distribution and the second distribution, whether the second plurality of cells expressing the candidate antibody exhibits the novel epitope specificity or a same epitope specificity as one or more of the first plurality of cells.

In some embodiments, the comparison between the first distribution and the second distribution is performed by applying a Kolmogorov-Smirnov test and/or an outlier detection algorithm.

In some embodiments, the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

Further provided herein is a system comprising at least one data processor; and at least one memory storing instructions, which when executed by the at least one data processor, result in operations comprising: generating, based at least on a first dataset indicating a first epitope specificity of a first plurality of cells expressing a known antibody, a first reduced dimension representation of the first dataset; generating, based at least on a second dataset indicating a second epitope specificity of a second plurality of cells expressing a candidate antibody, a second reduced dimension representation of the second dataset; and determining, based at least on the first reduced dimension representation and the second reduced dimension representation, whether the second plurality of cells exhibit a novel epitope specificity or an epitope specificity of the epitope bin associated with the first plurality of cells.

In some embodiments, the first reduced dimension representation is generated by decomposing a first matrix comprising the first dataset, and wherein the second reduced dimension representation is generated by decomposing a second matrix comprising the second dataset. In some embodiments, the first matrix and/or the second matrix are decomposed by applying a principle component analysis (PCA), a neighborhood component analysis, a linear discriminant analysis, and/or a non-negative matrix factorization. In some embodiments, the first reduced dimension representation and the second reduced dimension representation is generated by embedding a reduced dimensional space. In some embodiments, the reduced dimensional space is embedded by applying a t-distributed stochastic neighbor embedding (t-SNE), a uniform manifold approximation and projection (UMAP), and/or a generalized linear model principle component analysis (GLMPCA).

In some embodiments, each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

In some embodiments, each element in the first matrix comprises a count of each of the first plurality of cells bound to the antigen, and wherein each element in the second matrix comprises a count of each of the second plurality of cells bound to the antigen.

In some embodiments, the antigen is labeled with a reporter oligonucleotide.

In some embodiments, the reporter oligonucleotide comprises at least one functional sequence, at least one common barcode, at least one capture sequence, and at least one UMI.

In some embodiments, the antigen is selected from the group consisting of a protein, a viral-like particle, and a nanoparticle. In certain embodiments, the antigen is a protein.

In some embodiments, the antigen comprises a point mutation. In certain embodiments, the point mutation alters the epitope specificity of the known antibody.

In some embodiments, the first reduced dimension representation and the second reduced dimension representation are generated by applying a graph-based embedding and reduction technique.

In some embodiments, at least one of the first dataset and the second dataset include one or more negative control antigens.

In some embodiments, the first reduced dimension representation includes a first cluster of cells corresponding to the first plurality of cells, and wherein the second reduced dimension representation includes a second cluster of cells corresponding to the second plurality of cells. In some embodiments, whether the second plurality of cells exhibit the novel epitope specificity or the epitope specificity associated with the first plurality of cells is determined based at least on a distribution of the second cluster of cells relative to the first cluster of cells.

In some embodiments, the system provided herein further comprises applying a clustering algorithm to validate the distribution of the second cluster of cells relative to the first cluster of cells.

In some embodiments, the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

In some embodiments, the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

In some embodiments, the system provided herein further comprises determining, for the first plurality of cells expressing the known antibody, a first plurality of scores representative of an epitope binding property of each of the first plurality of cells; determining, for the second plurality of cells expressing the candidate antibody, a second plurality of scores representative of the epitope binding property of each of the second plurality of cells; generating a first distribution of the first plurality of scores and a second distribution of the second plurality of scores; and determining, based at least on a comparison between the first distribution and the second distribution, whether the second plurality of cells expressing the candidate antibody exhibits the novel epitope specificity or a same epitope specificity as one or more of the first plurality of cells.

In some embodiments, the comparison between the first distribution and the second distribution is performed by applying a Kolmogorov-Smirnov test and/or an outlier detection algorithm.

In some embodiments, the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

In some embodiments, the first dataset and the second dataset are generated by at least: engineering the first plurality of cells to express the known antibody and the second plurality of cells to express the candidate antibody; incubating the antigen with the first plurality of cells and the second plurality of cells, wherein the antigen is labeled with the reporter oligonucleotide; generating a plurality of single cell suspensions, wherein each of the plurality of single cell suspensions comprises one of either the first plurality of cells or the second plurality of cells bound to or not bound to the antigen; and generating a library of barcoded nucleic acid molecules from the first plurality of cells and the second plurality of cells, wherein the library of barcoded nucleic acid molecules comprises the reporter sequence or complement thereof and sequences corresponding to immune receptors; and sequencing the library of barcoded nucleic acid molecules.

Also provided herein is an antibody identified by the system disclosure herein. In some embodiments, the antibody is identified based at least on the second cluster of cells exhibiting the novel epitope specificity or the epitope specificity of the epitope bin associated with the first cluster of cells.

In some embodiments, the antibody is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody. In certain embodiments, the antibody is an oligoclonal antibody.

The present disclosure also provides a computer-implemented method. In some embodiments, the computer-implemented method comprises generating, based at least on a first dataset indicating a first epitope specificity of a first plurality of cells expressing a known antibody, a first reduced dimension representation of the first dataset; generating, based at least on a second dataset indicating a second epitope specificity of a second plurality of cells expressing a candidate antibody, a second reduced dimension representation of the second dataset; and determining, based at least on the first reduced dimension representation and the second reduced dimension representation, whether the second plurality of cells exhibit a novel epitope specificity or an epitope specificity of the epitope bin associated with the first plurality of cells.

In some embodiments, the first reduced dimension representation is generated by decomposing a first matrix comprising the first dataset, and wherein the second reduced dimension representation is generated by decomposing a second matrix comprising the second dataset.

In some embodiments, the first matrix and/or the second matrix are decomposed by applying a principle component analysis (PCA), a neighborhood component analysis, a linear discriminant analysis, and/or a non-negative matrix factorization.

In some embodiments, the first reduced dimension representation and the second reduced dimension representation is generated by embedding a reduced dimensional space.

In some embodiments, the reduced dimensional space is embedded by applying a t-distributed stochastic neighbor embedding (t-SNE), a uniform manifold approximation and projection (UMAP), and/or a generalized linear model principle component analysis (GLMPCA).

In some embodiments, each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

In some embodiments, each element in the first matrix comprises a count of each of the first plurality of cells bound to the antigen, and wherein each element in the second matrix comprises a count of each of the second plurality of cells bound to the antigen. In some embodiments, the antigen is labeled with a reporter oligonucleotide. In some embodiments, the reporter oligonucleotide comprises at least one functional sequence, at least one common barcode, at least one capture sequence, and at least one UMI.

In some embodiments, the antigen is selected from the group consisting of a protein, a viral-like particle, and a nanoparticle. In certain embodiments, the antigen is a protein.

In some embodiments, the antigen comprises a point mutation. In certain embodiments, the point mutation alters the epitope specificity of the known antibody.

In some embodiments, the first reduced dimension representation and the second reduced dimension representation are generated by applying a graph-based embedding and reduction technique.

In some embodiments, at least one of the first dataset and the second dataset include one or more negative control antigens. In some embodiments, the first reduced dimension representation includes a first cluster of cells corresponding to the first plurality of cells, and wherein the second reduced dimension representation includes a second cluster of cells corresponding to the second plurality of cells.

In some embodiments, whether the second plurality of cells exhibit the novel epitope specificity or the epitope specificity associated with the first plurality of cells is determined based at least on a distribution of the second cluster of cells relative to the first cluster of cells.

In some embodiments, the computer-implemented method further comprises applying a clustering algorithm to validate the distribution of the second cluster of cells relative to the first cluster of cells.

In some embodiments, the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

In some embodiments, the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

In some embodiments, the computer-implemented method further comprises determining, for the first plurality of cells expressing the known antibody, a first plurality of scores representative of an epitope binding property of each of the first plurality of cells; determining, for the second plurality of cells expressing the candidate antibody, a second plurality of scores representative of the epitope binding property of each of the second plurality of cells; generating a first distribution of the first plurality of scores and a second distribution of the second plurality of scores; and determining, based at least on a comparison between the first distribution and the second distribution, whether the second plurality of cells expressing the candidate antibody exhibits the novel epitope specificity or a same epitope specificity as one or more of the first plurality of cells.

In some embodiments, the comparison between the first distribution and the second distribution is performed by applying a Kolmogorov-Smirnov test and/or an outlier detection algorithm.

In some embodiments, the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

In some embodiments, the first dataset and the second dataset are generated by at least: engineering the first plurality of cells to express the known antibody and the second plurality of cells to express the candidate antibody; incubating the antigen with the first plurality of cells and the second plurality of cells, wherein the antigen is labeled with the reporter oligonucleotide; generating a plurality of single cell suspensions, wherein each of the plurality of single cell suspensions comprises one of either the first plurality of cells or the second plurality of cells bound to or not bound to the antigen; and generating a library of barcoded nucleic acid molecules from the first plurality of cells and the second plurality of cells, wherein the library of barcoded nucleic acid molecules comprises the reporter sequence or complement thereof and sequences corresponding to immune receptors; and sequencing the library of barcoded nucleic acid molecules.

In additional embodiments, the present disclosure provides an antibody identified by the system provided herein. In some embodiments, the antibody is identified based at least on the second cluster of cells exhibiting the novel epitope specificity or the epitope specificity of the epitope bin associated with the first cluster of cells. In some embodiments, the antibody is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody. In certain embodiments, the antibody is an oligoclonal antibody.

Further provided herein, among others, is a non-transitory computer readable medium storing instructions, which when executed by at least one data processor, result in operations comprising: generating, based at least on a first dataset indicating a first epitope specificity of a first plurality of cells expressing a known antibody, a first reduced dimension representation of the first dataset; generating, based at least on a second dataset indicating a second epitope specificity of a second plurality of cells expressing a candidate antibody, a second reduced dimension representation of the second dataset; and determining, based at least on the first reduced dimension representation and the second reduced dimension representation, whether the second plurality of cells exhibit a novel epitope specificity or an epitope specificity of the epitope bin associated with the first plurality of cells. Also provided herein is a method that includes (a) generating a plurality of single cell suspensions, wherein each of the plurality of single cell suspensions comprises one of either a first plurality of cells or a second plurality of cells bound to or not bound to the antigen; (b) generating a library of barcoded nucleic acid molecules from the first plurality of cells and the second plurality of cells, wherein the library of barcoded nucleic acid molecules comprises a reporter sequence or complement thereof and sequences corresponding to immune receptors; (c) sequencing the library of barcoded nucleic acid molecules; (d) obtaining a first epitope specificity of the first plurality of cells and a second epitope specificity of the second plurality of cells by contacting the antigen with the first plurality of cells and the second plurality of cells, wherein the antigen is labeled with a reporter oligonucleotide comprising the reporter sequence, and wherein the first plurality of cells is engineered to express a known antibody and the second plurality of cells is engineered to express a candidate antibody; (e) generating, i) a first reduced dimension representation of the first epitope specificity of the first plurality of cells expressing the known antibody; and ii) a second reduced dimension representation of the second epitope specificity of the second plurality of cells expressing the candidate antibody. Based at least on the first reduced dimension representation and the second reduced dimension representation, it is determined whether the second plurality of cells exhibit a novel epitope specificity or an epitope specificity associated with the first plurality of cells.

In some embodiments, generating a plurality of single cell suspensions comprises: contacting the antigen with the first plurality of cells and the second plurality of cells, and partitioning the first plurality of cells and the second plurality of cells into a plurality of partitions, wherein a partition of the plurality of partitions comprises a cell of the first plurality of cells or the second plurality of cells bound or not bound to the antigen, and a plurality of nucleic acid barcode molecules wherein a first nucleic acid barcode molecule of the plurality of nucleic acid barcode molecules comprises a partition barcode sequence.

In some embodiments, generating a library of barcoded nucleic acid molecules from the first plurality of cells and the second plurality of cells comprises a. in the partition, coupling the first nucleic acid barcode molecule to the reporter oligonucleotide, and b. using the reporter oligonucleotide coupled to the first nucleic acid barcode molecule to generate a first barcoded nucleic acid molecule comprising the reporter sequence or a reverse complement thereof and the partition barcode sequence or a reverse complement thereof.

In some embodiments, a second nucleic acid barcode molecule of the plurality of nucleic acid barcode molecules comprises the partition barcode sequence, and generating a library of barcoded nucleic acid molecules from the first plurality of cells and the second plurality of cells further comprises a. in the partition, coupling the second nucleic acid barcode molecule to a nucleic acid analyte of the cell bound to the antigen, the nucleic acid analyte comprising a sequence of an immune receptor expressed by the cell bound to the antigen, or a reverse complement thereof, and b. using the nucleic acid analyte of the cell bound to the antigen coupled to the second nucleic acid barcode molecule to generate a second barcoded nucleic acid molecule comprising the sequence of the immune receptor expressed by the cell bound to the antigen, or a reverse complement thereof.

In some embodiments, the plurality of nucleic acid barcode molecules is attached to a bead, and the partition barcode sequence identifies the bead. In some embodiments, the first nucleic acid barcode molecule comprises a first capture sequence configured to couple to the reporter oligonucleotide. In some embodiments, the reporter oligonucleotide further comprises a capture handle sequence complementary to the first capture sequence. In some embodiments, the second nucleic acid barcode molecule further comprises a second capture sequence configured to couple to the nucleic acid analyte of the cell bound to the antigen. In some embodiments, the nucleic acid analyte is an mRNA analyte or a cDNA molecule generated from the mRNA analyte. In some embodiments, the second capture sequence is a template switch oligonucleotide sequence. In some embodiments, the first capture sequence and the second capture sequence are identical. In some embodiments, the first capture sequence and the second capture sequence are different. In some embodiments, the partition is a droplet. In some embodiments, the partition is a well.

In some embodiments, the method further comprises contacting the first plurality of cells with a first cell group labeling agent comprising a first cell group reporter oligonucleotide, the first cell group reporter oligonucleotide comprising a first cell group reporter sequence that identifies the first plurality of cells, and contacting the second plurality of cells with a second cell group labeling agent, the second cell group labeling agent comprising a second cell group reporter oligonucleotide comprising a second cell group reporter sequence that identifies the second plurality of cells.

In some embodiments, a third nucleic acid barcode molecule of the plurality of nucleic acid barcode molecules comprises the partition barcode sequence, and (ii) generating a library of barcoded nucleic acid molecules from the first plurality of cells and the second plurality of cells comprises a. in the partition, coupling the third nucleic acid barcode molecule to the first cell group reporter oligonucleotide or to the second cell group reporter oligonucleotide, and b. using the third nucleic acid barcode molecule coupled to the first cell group reporter oligonucleotide or to the second cell group reporter oligonucleotide to generate a third barcoded nucleic acid molecule comprising the first cell group reporter sequence or the second cell group reporter sequence, or a reverse complement thereof, and the partition barcode sequence or a reverse complement thereof.

In some embodiments, the method further comprises determining a sequence of the first barcoded nucleic acid molecule or a derivative thereof, the second barcoded nucleic acid molecule or a derivative thereof, and/or the third barcoded nucleic acid molecule or a derivative thereof.

### BRIEF DESCRIPTION OF THE DRAWINGS

FIG. 1 shows an exemplary microfluidic channel structure for partitioning individual biological particles.

FIG. 2 shows an exemplary microfluidic channel structure for delivering barcode carrying beads to droplets.

FIG. 3 shows an exemplary microfluidic channel structure for the controlled partitioning of beads into discrete droplets.

FIG. 4 illustrates an example of partition-specific barcode molecules carrying bead.

FIG. 5 schematically illustrates an example microwell array.

FIG. 6 schematically illustrates an example workflow for processing nucleic acid molecules.

FIG. 7 illustrates another example of partition-specific barcode molecules carrying bead.

FIG. 8A shows an example of a reporter oligonucleotide conjugated to an antigen (or an antibody) hybridized to a corresponding partition-specific barcode molecule conjugated to a bead.

FIG. 8B shows an example of molecules that can be derived from a cell (such as RNA molecules) that can be processed to append a partition-specific barcode sequence to the molecule.

FIG. 8C shows an example of a partition-specific barcode molecule conjugated to a bead, wherein the partition-specific barcode molecule hybridizes to an mRNA molecule.

FIG. 9 shows an example of a reporter oligonucleotide conjugated to an antigen (including a protein (910), an antibody (920), or an WIC multimer (930)).

FIG. 10 illustrates an example of partition-specific barcode molecules carrying bead.

FIG. 11 depicts a system diagram illustrating an example of an analysis system, in accordance with some example embodiments.

FIG. 12A depicts an example of data representative of an epitope specificity of known antibodies and candidate antibodies, in accordance with some example embodiments.

FIG. 12B depicts an example of a count matrix, in accordance with some example embodiments.

FIG. 12C depicts an example of a visualization an epitope specificity of various antibody expressing cells, in accordance with some example embodiments.

FIG. 12D depicts another example of a visualization of an epitope specificity of various cells, in accordance with some example embodiments.

FIG. 13 depicts a block diagram illustrating an example of a computer system, in accordance with some example embodiments.

FIG. 14 depicts a flowchart illustrating an example of a process for determining epitope specificity, in accordance with some example embodiments.

### DETAILED DESCRIPTION OF THE DISCLOSURE

The present disclosure generally relates to methods and systems for identifying epitopes targeted by any antibody-based therapy. In one aspect, the present disclosure relates to a method for determining whether a second set cells exhibits a novel epitope specificity or an epitope specificity associated with a set of cells. In another aspect, the present disclosure generally relates to methods and systems for obtaining epitope specify of candidate antibodies against one or more known antibodies. Further, the present disclosure also provides systems and methods for epitope binning.

The following description and examples illustrate embodiments of the present disclosure in detail. It is to be understood that the present disclosure is not limited to the particular embodiments described herein and as such can vary. Those of skill in the art will recognize that there are variations and modifications of the present disclosure, which are encompassed within its scope.

Unless defined otherwise, all technical and scientific terms used herein have the same meaning as commonly understood by one of ordinary skill in the art to which the disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time,” as used herein, can refer to a response time of less than about 1 second, a tenth of a second, a hundredth of a second, a millisecond, or less. The response time can be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals can include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic materials from a subject, which can be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together can constitute a human genome.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

As used herein, the term “barcoded nucleic acid molecule” generally refers to a nucleic acid molecule that results from, for example, the processing of a partition-specific barcode molecule with a reporter oligonucleotide. For example, in the methods and systems described herein, hybridization and reverse transcription of a reporter oligonucleotide or a messenger RNA (mRNA) molecule of a cell with a partition-specific barcode molecule results in a barcoded nucleic acid molecule that has a sequence corresponding to the nucleic acid sequence of the reporter oligonucleotide or a complement thereof or mRNA or a complement thereof and the partition-specific barcode or a complement thereof. A barcoded nucleic acid molecule can serve as a template, such as a template polynucleotide, that can be further processed (e.g., amplified) and sequenced to obtain the target nucleic acid sequence. For example, in the methods and systems described herein, a barcoded nucleic acid molecule can be further processed (e.g., amplified) and sequenced to obtain the nucleic acid sequence of the mRNA.

The term “sample,” as used herein, generally refers to a biological sample of a subject. The biological sample can include any number of macromolecules, for example, cellular macromolecules. The sample can be a cell sample. The sample can be a cell line or cell culture sample. The sample can include one or more cells. The sample can include one or more microbes. The biological sample can be a nucleic acid sample or protein sample. The biological sample can also be a carbohydrate sample or a lipid sample. The biological sample can be derived from another sample. The sample can be a tissue sample, such as a biopsy, core biopsy, needle aspirate, or fine needle aspirate. The sample can be a fluid sample, such as a blood sample, urine sample, or saliva sample. The sample can be a skin sample. The sample can be a cheek swab. The sample can be a plasma or serum sample. The sample can be a cell-free or cell free sample. A cell-free sample can include extracellular polynucleotides. Extracellular polynucleotides can be isolated from a bodily sample that can be selected from the group consisting of blood, plasma, serum, urine, saliva, mucosal excretions, sputum, stool, and tears.

The term “biological particle,” as used herein, generally refers to a discrete biological system derived from a biological sample. The biological particle can be a macromolecule. The biological particle can be a small molecule. The biological particle can be a virus. The biological particle can be a cell or derivative of a cell. The biological particle can be an organelle. The biological particle can be a rare cell from a population of cells. The biological particle can be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle can be a constituent of a cell. The biological particle can be or can include DNA, RNA, organelles, proteins, or any combination thereof. The biological particle can be or can include a matrix (e.g., a gel or polymer matrix) comprising a cell or one or more constituents from a cell (e.g., cell bead), such as DNA, RNA, organelles, proteins, or any combination thereof, from the cell. The biological particle can be obtained from a tissue of a subject. The biological particle can be a hardened cell. Such hardened cell may or may not include a cell wall or cell membrane. The biological particle can include one or more constituents of a cell, but may not include other constituents of the cell. An example of such constituents is a nucleus or an organelle. A cell can be a live cell. The live cell can be cultured, for example, when enclosed in a gel or polymer matrix, or cultured when comprising a gel or polymer matrix. In some embodiments, the biological particle is a cell engineered to express an antigen binding molecule, such as an antibody or an antigen binding fragment thereof. In some embodiments, the antibody is a known antibody. In some embodiments, the antibody is a candidate antibody. In some embodiments, the biological particle is a cell expressing a known antibody or a candidate antibody, bound or unbound by an antigen.

The term “sequencing,” as used herein, generally refers to methods and technologies for determining the sequence of nucleotide bases in one or more polynucleotides (e.g., barcoded nucleic acid molecules). The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing can be performed using nucleic acid amplification, polymerase chain reaction (PCR) (e.g., digital PCR, quantitative PCR, or real time PCR), or isothermal amplification. Such systems can provide a plurality of raw genetic data corresponding to the genetic information of a subject (e.g., human), as generated by the systems from a sample provided by the subject. In some examples, such systems provide sequencing reads (also “reads” herein). A read can include a string of nucleic acid bases corresponding to a sequence of e.g., a barcoded nucleic acid molecule that has been sequenced. In some situations, systems and methods provided herein can be used with proteomic information.

The term “macromolecular constituent,” as used herein, generally refers to a macromolecule contained within or from a biological particle. The macromolecular constituent can include a nucleic acid. In some cases, the biological particle can be a macromolecule. The macromolecular constituent can include DNA. The macromolecular constituent can include RNA. The RNA can be coding or non-coding. The RNA can be messenger RNA (mRNA), ribosomal RNA (rRNA) or transfer RNA (tRNA), for example. The RNA can be a transcript. The RNA can be small RNA that are less than 200 nucleic acid bases in length, or large RNA that are greater than 200 nucleic acid bases in length. Small RNAs can include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA can be double-stranded RNA or single-stranded RNA. The RNA can be circular RNA. The macromolecular constituent can include a protein. The macromolecular constituent can include a peptide. The macromolecular constituent can include a polypeptide.

The term “molecular tag,” as used herein, generally refers to a molecule capable of binding to a macromolecular constituent. The molecular tag can bind to the macromolecular constituent with high affinity. The molecular tag can bind to the macromolecular constituent with high specificity. The molecular tag can include a nucleotide sequence. The molecular tag can include a nucleic acid sequence. The nucleic acid sequence can be at least a portion or an entirety of the molecular tag. The molecular tag can be a nucleic acid molecule or can be part of a nucleic acid molecule. The molecular tag can be an oligonucleotide or a polypeptide. The molecular tag can include a DNA aptamer. The molecular tag can be or include a primer. The molecular tag can be, or include, a protein. The molecular tag can include a polypeptide. The molecular tag can be a barcode.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

The term “microwell,” as used herein, generally refers to a well with a volume of less than 1 mL. Microwells may be made in various volumes, depending on the application. For example, microwells may be made in a size appropriate to accommodate any of the partition volumes described herein.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods for Obtaining Epitope Specificity

One aspect of the present disclosure generally relates to methods and systems for obtaining epitope specify of candidate antibodies against one or more known antibodies. In some embodiments, the methods comprise labeling of an antigen (such as protein, viral-like particle, or nanoparticle, etc.) with a reporter oligonucleotide. In some embodiments, the methods comprise engineering cells, such as Ramos and other B cell lines, to express antibodies with known antigen specificity and/or known epitope specificity. In some embodiments, the methods comprise mixing of two types of engineered cells, such as engineered B cells: the engineered cells expressing the known antibodies and the engineered cells expressing candidate antibodies. In some embodiments, the methods comprise partitioning single cell suspensions. In some embodiments, control cells are spiked in or control sequences with known specificity are evaluated in light of an individual's immune history. In some embodiments, the methods comprise contacting of the resulting single cell suspension with the target antigen(s) of interest, including point mutants of the antigen labeled with unique reporter oligonucleotides and negative control antigens or antigens that are not expected to be bound by any of the cells in solution. Control antigens can also be antigens that are non-overlapping or partially overlapping (i.e. sufficient structural separation to make recognition by one antibody impossible).

In some embodiments, the methods comprise preparing nucleic acid libraries corresponding to target antigen(s) of interest. These nucleic acid libraries comprise barcoded nucleic acid molecules which comprise reporter sequences from reporter oligonucleotides that were conjugated to the target antigens. The nucleic acid libraries may further comprise sequences corresponding to immune receptors, including V(D)J/B cell receptors. In further embodiments, the methods comprise mathematical analysis to identify similarities and differences between feature vectors of known antibody B cells from candidate antibody B cells.

In some embodiments, the methods provided herein comprise obtaining a first epitope specificity of a first plurality of cells and a second epitope specificity of a second plurality of cells. In some embodiments, the epitope specificities are obtained by contacting an antigen with the first plurality of cells and the second plurality of cells. In some embodiments, the methods further comprise engineering the first plurality of cells to express a first antibody and the second plurality of cells to express a second antibody. In some other embodiments, the first antibody is a known antibody. In other embodiments, the second antibody is a candidate antibody.

As used herein, a candidate antibody generally refers to an antibody of which the epitope binding specificity is unknown. In some embodiments, the epitope binding specificity of the candidate antibody can be determined by the methods and systems provided herein. As used herein, a known antibody generally refers to an antibody of which the epitope binding specificity is known.

In other embodiments, the first plurality of cells is engineered to express the first antibody (e.g., a known antibody) and the second plurality of cells are engineered to express the second antibody (e.g., a candidate antibody). Methods for engineering cell to express a target gene or protein are generally known in the art. Exemplary methods for genetic engineering include, without limitations, plasmid or vector mediated gene delivery, CRISPR-mediated genome editing, transcription activator-like effector nucleases (TALEN), and Zinc-finger nucleases (ZFNs).

### Antigens

An antigen encompassed by the present disclosure can include, but is not limited to, a protein, a peptide, an antibody (or an epitope binding fragment thereof), a lipophilic moiety (such as cholesterol), a cell, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, a viral-like particle, a nanoparticle, or any combination thereof. In some specific embodiments, the antigen comprises a protein, a viral-like particle, and a nanoparticle. In an exemplary embodiment, the antigen is a protein. In some embodiments, the antigen is a cell. In some embodiments, the antigen is a cell expressing a target. In some embodiments, the antigen is a target expressed by a cell.

An antigen can be a molecule that can have affinity to an antigen binding molecule. For example, an antigen can have affinity to an antibody or antigen binding fragment thereof. In some embodiments, when contacted with an antigen binding molecule, the antigen can bind to the antigen binding molecule. In some embodiments, an antigen can be a biomolecule, such as a biologic therapeutic molecule. Examples of biologic therapeutic molecules can be, for example, a drug-reactive antibody or anti-drug antibody that is produced from a living organism or that contains one or more components of a living organism. A biologic therapeutic molecule can be derived from a human, animal, or microorganism using biotechnology techniques. Examples of biologic therapeutic molecules can include, for example, an immunological molecule (e.g. an antibody (such as a monoclonal antibodies), a fusion protein, a protein product of a gene therapy, a peptide, or other biologic molecule.

In some embodiments, an antigen can be an antibody or antigen binding fragment thereof. In some embodiments, an antigen can be an antibody-drug conjugate. In some embodiments, an antigen can be a therapeutic antibody or antigen binding fragment thereof (e.g., a monoclonal antibody).

### Epitope Modification

In some embodiments, the methods provided herein can include modifying an epitope of the antigen. In some embodiments, the antigen comprises one or more point mutations. In some embodiments, the modification, e.g., the one or more point mutations, can change the epitope specificity of the first antibody (e.g., a known antibody). In some embodiments, an epitope can be modified to reduce the affinity of the antigen binding molecule for the antigen. In some such cases, an epitope can be modified to partially or fully prevent binding of the antigen binding molecule to the antigen.

In some embodiments, modification of an epitope can be based on the identity of the antigen binding molecule or the epitope. In some cases, an epitope determined, for example by epitope mapping as provided herein, can be modified. After modification of the epitope, the modified antigen can be contacted with the antigen binding molecule, and affinity between the modified antigen and antigen binding molecule can be determined. In some embodiments, the affinity between the modified antigen and antigen binding molecule can be significantly reduced or enhanced, or physiologically negligible.

Epitope modification can include one or more of: a modification (e.g., insertion, deletion, or mutation) of the amino acid sequence of the epitope, a post-translational modification to the epitope (e.g., glycosylation, ubiquitination, phosphorylation, myristoylation, palmitoylation, isoprenylation, farnesylation, geranylgeranylation, glipyatyon, lipoylation, attachment of a flavin moiety, attachment of heme C, phosphopantetheinylation, retinylidene Schiff base formation, diphthamide formation, ethanolamide phosphoglycerol attachment, hypusine formation, beta-Lysine addition, acylation, alkylation, amidation, amide bond formation, arginylation, polyglutamylation, polyglycylation, butyrylation, gamma-carboxylation, malonylation, hydroxylation, iodination, nucleotide addition, phosphate ester or phosphoramidate formation, adenylation, uridylylation, propionylation, pyroglutamate formation, S-glutathionylation, S-nitrosylation, S-sulfenylation, S-sulfinylation, succinylation, sulfation, glycation, carbamylation, carbonylation, spontaneous isopeptide bond formation, biotinylation, carbamylation, oxidation, pegylation, ISGylation, SUMOylation, neddylation, pupylation, citrullination, deamidation, eliminylation, formation of a disulfide bridge, proteolytic cleavage, isoaspartate formation, racemization, protein splicing, or a combination thereof), or binding of a molecule to the epitope to block the antigen binding molecule from binding the epitope. Epitope modification can further include chemical alterations, e.g., chemical alterations, of one or more amino acids of the epitope, truncations, deletions, insertions, point substitutions. Further, epitope modification can include fragments of an antigen. Additionally, epitopes can also be modified through multimerization-based masking. For example, monomeric, dimeric, trimeric, tetrameric, pentameric, and heptameric versions of the same antigen each have different residues presented in the final molecule.

In some embodiments, one or more antigens having one or more modified epitopes (e.g., antigen variants) can be conjugated to reporter oligonucleotides and used as antigens in methods provided herein. In some embodiments, antigens having one or more modified epitopes can have reduced affinity for the antigen binding molecule. In some embodiments, antigens having one or more modified epitopes can have no affinity for the antigen binding molecule. In other embodiments, antigens having one or more modified epitopes can have increased affinity for the antigen binding molecule.

Labeling Antigens with Reporter Oligonucleotide

In the methods and systems described herein, one or more antigens capable of binding to or otherwise coupling to the known antibodies or the candidate antibodies can be used to characterize the epitope specificity of the candidate antibodies. In some instances, the epitope specificity is characterized by cell surface features of the cells expressing the known antibodies and/or the candidate antibodies. In some instances, cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof.

The term “barcode,” as used herein, generally refers to a label, or identifier, that conveys or is capable of conveying information about an analyte. A barcode can be part of an analyte. A barcode can be independent of an analyte. A barcode can be attached to an analyte (e.g., a reporter oligonucleotide). A barcode can be a combination of identifiers in addition to an endogenous characteristic of the analyte (e.g., size of the analyte or end sequence(s)). A barcode can be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can facilitate identification and/or quantification of individual sequencing-reads.

A labelling agent (e.g., an antigen) can contain (e.g., is attached to) a reporter oligonucleotide that is indicative of the cell surface feature to which the binding group binds. For example, the reporter oligonucleotide can contain a reporter barcode (e.g., a barcode sequence) that permits identification of the labelling agent. For example, an agent (e.g., antigen) that is specific to one type of cell feature (e.g., a first cell surface feature) can have coupled thereto a first reporter oligonucleotide, while an antigen (e.g., antigen) that is specific to a different cell feature (e.g., a second cell surface feature) can have a different reporter oligonucleotide coupled thereto. For a description of exemplary methods of labeling agents (e.g., antigens) with reporter oligonucleotides containing reporter barcodes (e.g., barcode sequences), and methods of use, see, e.g., U.S. Pat. No. 10,550,429; U.S. Pat. Pub. 20190177800; and U.S. Pat. Pub. 20190367969, which are each incorporated by reference herein in their entirety.

In a particular example, a library of potential cell feature labelling agents (e.g., antigens) can be provided associated with reporter oligonucleotides, e.g., where a different reporter oligonucleotide sequence is associated with each labelling agent (e.g., antigen) capable of binding to a specific cell feature. In some other aspects, different members of the library can be characterized by the presence of a different reporter oligonucleotide sequence label, e.g., an antibody capable of binding to a first type of protein may have associated with it a first known oligonucleotide sequence, while an antibody capable of binding to a second protein (e.g., different than the first protein) may have a second known oligonucleotide sequence associated with it.

The cells can be incubated with the library of labelling agents (e.g., antigens) conjugated with reporter oligonucleotides that can represent labelling agents (e.g., antigens) to a broad panel of different cell features, e.g., receptors, proteins, etc., and which include their associated reporter oligonucleotides. Unbound labelling agents (e.g., antigens) can be washed from the cells. The cells can then be co-partitioned (e.g., into droplets or wells) along with partition-specific barcode molecules (e.g., attached to a bead, such as a gel bead). As a result, the partitions can include the cell or cells, as well as the bound labelling agents (e.g., antigens) and their known, associated reporter oligonucleotides with reporter barcodes (e.g., barcode sequences) and partition-specific barcode molecules with partition-specific barcodes.

In other instances, e.g., to facilitate sample multiplexing, an antigen that is specific to a particular cell feature can have a first plurality of the antigen (e.g., an antibody or lipophilic moiety) coupled to a first reporter oligonucleotide with a first reporter barcode (e.g., a barcode sequence) and a second plurality of the antigen coupled to a second reporter oligonucleotide with a second reporter barcode (e.g., a barcode sequence). In this way, different samples or groups can be independently processed and subsequently combined together for pooled analysis (e.g., partition-based barcoding as described elsewhere herein). See, e.g., U.S. Pat. Pub. 20190323088, which is hereby incorporated by reference its entirety.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some aspects, these reporter oligonucleotides with reporter barcodes (e.g., barcode sequences) can contain nucleic acid barcode sequences that permit identification of the labelling agent (e.g., antigen) which the reporter oligonucleotide is coupled to. The selection of oligonucleotides with barcodes as a reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the oligonucleotides to the labelling agents (e.g., antigens) can be achieved through any of a variety of direct or indirect, covalent or non-covalent associations or attachments. For example, oligonucleotides can be covalently attached to a portion of an agent (e.g., antigen) (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labelling agents (e.g., antigens). Commercially available kits, such as those from Thunderlink and Abcam, and techniques common in the art can be used to couple reporter oligonucleotides with reporter barcodes (e.g., barcode sequences) to labelling agents (e.g., antigens) as appropriate. In some example, a labelling agent (e.g., antigen) is indirectly (e.g., via hybridization) coupled to a partition-specific barcode molecule. For instance, the labelling agent (e.g., antigen) can be directly coupled (e.g., covalently bound) to a reporter oligonucleotide that comprises a sequence that hybridizes with a sequence of the partition-specific barcode molecules. Hybridization of the partition-specific barcode molecules to the reporter oligonucleotide couples the labelling agent (e.g., antigen) to the partition-specific barcode molecules. In some embodiments, the reporter oligonucleotides are releasable from the labelling agent (e.g., antigen), such as upon application of a stimulus. For example, the reporter oligonucleotide can be attached to the labelling agent (e.g., antigen) through a labile bond (e.g., chemically labile, photolabile, thermally labile, etc.) as generally described for releasing molecules from supports elsewhere herein. In some instances, the reporter oligonucleotides and/or the partition-specific barcode molecules described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., antigen) can include a reporter oligonucleotide with a reporter barcode (e.g., a barcode sequence) and a tag. A tag can be an enzyme, a fluorophore, a quantum dot, a covalently or non-covalently attached protein or peptide, a carbohydrate, a radioisotope, a molecule capable of a colorimetric reaction, a magnetic particle, a small molecule, or any other suitable molecule or compound capable of detection. The tag can be conjugated to a labelling agent (or a reporter oligonucleotide with a reporter barcode, such as a barcode sequence) either directly or indirectly (e.g., the tag can be conjugated to a molecule that can bind to the labelling agent or the reporter oligonucleotide).

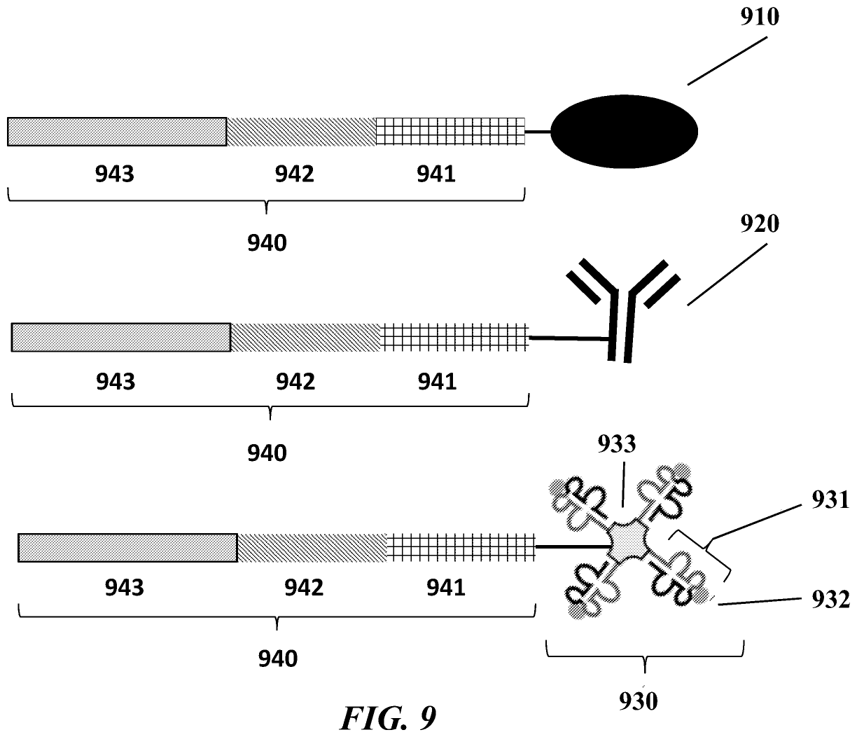

FIG. 9 describes exemplary agents (e.g., antigens) (910, 920, 930) conjugated to a reporter oligonucleotide (940) attached thereto. The labelling agent (e.g., antigen) 910, 920, or 930 is attached (either directly, e.g., covalently attached, or indirectly) to a reporter oligonucleotide 940. A reporter oligonucleotide 940 can contain a reporter barcode sequence 942 that identifies the labelling agent (e.g., antigen) 910, 920, or 930. A reporter oligonucleotide 940 can also contain one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 9, in some instances, reporter oligonucleotide 940 conjugated to a labelling agent (e.g., antigen) (e.g., 910, 920, 930) can include a functional sequence 941 (e.g., an adaptor), a barcode sequence that identifies the labelling agent (e.g., antigen) (e.g., 910, 920, 930), and functional sequence (e.g., adaptor) 943. Capture handle 943 can be configured to hybridize to a complementary sequence (e.g., a capture sequence), such as a complementary sequence (e.g., capture sequence) present on a partition-specific barcode molecules (not shown), such as those described elsewhere herein. A capture handle can include a sequence that is complementary to a capture sequence on a partition-specific barcode molecule. In some instances, a partition-specific barcode molecule is attached to a support (e.g., a bead, such as a gel bead), such as those described elsewhere herein. For example, partition-specific barcode molecules can be attached to the support via a releasable linkage (e.g., comprising a labile bond), such as those described elsewhere herein. In some instances, a reporter oligonucleotide 940 includes one or more additional functional sequences, such as those described above.

In some instances, labelling agent (e.g., antigen) 910 is a protein or polypeptide (e.g., an antigen or prospective antigen) conjugated to reporter oligonucleotide 940. Reporter oligonucleotide 940 contains reporter barcode sequence 942 that identifies protein or polypeptide 910 and can be used to infer the presence of, e.g., a binding partner of protein or polypeptide 910 (e.g., a molecule or compound to which the protein or polypeptide binds). In some instances, 910 is a lipophilic moiety (e.g., cholesterol) comprising reporter oligonucleotide 940, where the lipophilic moiety is selected such that 910 integrates into a membrane of a cell or nucleus. Reporter oligonucleotide 940 contains reporter barcode sequence 942 that identifies lipophilic moiety 910 which in some instances is used to tag cells (e.g., groups of cells, cell samples, etc.) for multiplex analyses as described elsewhere herein.

In some instances, the labelling agent (e.g., antigen) is an antibody 920 (or an epitope binding fragment thereof) including reporter oligonucleotide 940. Reporter oligonucleotide 940 includes reporter barcode sequence 942 that identifies antibody 920 and can be used to infer the presence of, e.g., a target of antibody 920 (e.g., a molecule or compound to which antibody 920 binds).

In other embodiments, labelling agent (e.g., antigen) 930 includes an MHC molecule 931 including peptide 932 and oligonucleotide 940 that identifies peptide 932. In some instances, the MHC molecule is coupled to a support 933. In some instances, support 933 is streptavidin (e.g., MHC molecule 931 can include biotin). In other embodiments, support 933 is a polysaccharide, such as dextran. In some instances, reporter oligonucleotide 940 can be directly or indirectly coupled to MHC labelling agent 930 in any suitable manner, such as to MCH molecule 931, support 933, or peptide 932. In some embodiments, labelling agent 930 includes a plurality of MHC molecules, e.g., is an MHC multimer, which can be coupled to a support (e.g., 933). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labeling of various labelling agents (e.g., antigens), including antibody and MHC-based labelling agents, reporter oligonucleotides, and methods of use, see, e.g., U.S. Pat. No. 10,550,429 and U.S. Pat. Pub. 20190367969, which are each incorporated by reference herein in their entirety.

### Oligonucleotides

An oligonucleotide can be a molecule which can be a chain of nucleotides. Oligonucleotides described herein can include ribonucleic acids. Oligonucleotides described herein can include deoxyribonucleic acids. In some cases, oligonucleotides can be of any sequence, including a user-specified sequence.

In some embodiments, an oligonucleotide can include G, A, T, U, C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. An oligonucleotide can include an LNA, a PNA, a UNA, or a morpholino oligomer, for example. The oligonucleotides used herein can contain natural or non-natural nucleotides or linkages.

An oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, an oligonucleotide can be between 10-30, between 10-50, between 10-between 10-100, between 20-50, between 20-70, between 20-100, between 30-50, between 30-70, between 30-100, between 40-70, between 40-100, between 50-70, between between 60-70, between 60-80, between 60-90, or between 60-100 nucleotides in length. In some cases, an oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, an oligonucleotide can be wholly single stranded. In some cases, an oligonucleotide can be partially double stranded. A partially double stranded region can be at the 3′ end of the oligonucleotide, at the 5′ end of the oligonucleotide, or between the 5′ end and 3′ end of the oligonucleotide. In some cases, there can be more than one double stranded region.

In some cases, an oligonucleotide can have a secondary structure. In some cases, an oligonucleotide can have a tertiary structure. Some oligonucleotides can have a structure such that it can fold on itself (e.g. if one region of the oligonucleotide is complementary to another region of the oligonucleotide) to produce one or more double stranded regions comprising a single strand.

In some cases, a segment of an oligonucleotide able to bind a circular nucleic acid primer can be exposed in a single stranded region of the oligonucleotide or an unfolded region of the oligonucleotide. In some cases, a segment of an oligonucleotide able to bind a circular nucleic acid primer can be in a double stranded or folded region of the oligonucleotide, such that upon melting of the oligonucleotide, such a circular nucleic acid primer can bind.

An oligonucleotide can include a sequence that can be used to isolate or identify the antigen. For example, an oligonucleotide can include a barcode sequence (e.g., a reporter barcode, such as a barcode sequence, and/or a partition-specific barcode). In some embodiments, different reporter oligonucleotides (e.g., reporter oligonucleotides associated with different labelling agents (e.g., antigens) can contain different reporter barcode sequences. A reporter barcode sequence or other feature of a reporter oligonucleotide can be configured to be utilized to identify or isolate the reporter oligonucleotide and/or a labelling agent (e.g., antigen) associated with the reporter oligonucleotide. For example, identification or isolation can be achieved through base pairing, amplification, sequencing, library creation, imaging (e.g., fluorescent imaging of a label conjugated to the oligonucleotide, for example via the barcode), or other methods.

An oligonucleotide can include any nucleic acid based feature provided herein.

In some cases, the nucleic acid molecule (e.g., a partition-specific barcode molecules) can contain one or more functional sequences, for example, for attachment to a sequencing flow cell, such as, for example, a P5 sequence (or a portion thereof) for Illumina® sequencing. In some cases, the nucleic acid molecule or derivative thereof (e.g., partition-specific barcode molecules) can contain one or more additional functional sequences, such as, for example, a P7 sequence (or a portion thereof) for attachment to a sequencing flow cell for Illumina sequencing. In some cases, the nucleic acid molecule (e.g., a partition-specific barcode molecule) can contain a partition-specific barcode sequence. In some cases, the nucleic acid molecule (e.g., a partition-specific barcode molecule) can further include a unique molecular identifier (UMI). In some cases, the nucleic acid molecule (e.g., a partition-specific barcode molecule) can contain an R1 primer sequence for Illumina sequencing. In some cases, the nucleic acid molecule (e.g., a partition-specific barcode molecule) can contain an R2 primer sequence for Illumina sequencing. In some cases, a functional sequence can include a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence can contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such nucleic acid molecules (e.g., partition-specific barcode molecules) and uses thereof, as can be used with compositions, devices, methods and systems of the present disclosure, are provided in U.S. Patent Pub. Nos. 2014/0378345 and 2015/0376609, each of which is entirely incorporated herein by reference.

### Conjugation

A reporter oligonucleotide can be coupled to a labelling agent (e.g., antigen) provided herein. Coupling can include a physical or spatial association between the reporter oligonucleotide and the labelling agent (e.g., antigen). In some aspects, coupling can include conjugating the labelling agent (e.g., antigen) to the reporter oligonucleotide.

A labelling agent (e.g., antigen) (e.g., a therapeutic antibody or antibody drug complex) can be conjugated to a reporter oligonucleotide. The reporter oligonucleotide can be conjugated anywhere along the amino acid chain of the labelling agent (e.g., antigen). In some embodiments, the reporter oligonucleotide can be conjugated to the N terminus, the C terminus, or between the N terminus and the C terminus of the labelling agent (e.g., antigen).

In some embodiments, the reporter oligonucleotide conjugated to the antigen does not interfere with binding of the antigen to an antigen binding molecule. A reporter oligonucleotide can be conjugated away from a site on the antigen that binds to the antigen binding molecule (e.g., an epitope). In some embodiments, such as when the binding site is unknown, a reporter oligonucleotide can be conjugated to different parts of the antigen on different copies of the antigen.

Either end (e.g., the 3′ end or the 5′ end) of the reporter oligonucleotide can be conjugated to the labelling agent (e.g., antigen). In some embodiments, more than one reporter oligonucleotide can be conjugated to the labelling agent (e.g., antigen).

Conjugation of a reporter oligonucleotide to a labelling agent (e.g., antigen) can preserve the tertiary and/or quaternary structure of the labelling agent (e.g., antigen). In some embodiments, the structure of the labelling agent (e.g., antigen) can be completely preserved. In some embodiments, the structure of a binding site (e.g., a site where the labelling agent (e.g., antigen) can bind to an antigen binding molecule such as an antibody) can be preserved. In some embodiments, the location and/or orientation of surface residues of the labelling agent (e.g., antigen) can be preserved.

In some embodiments, the link between a labelling agent (e.g., antigen) and a reporter oligonucleotide can be stable. Stability can be, for example, under physiological conditions (e.g., physiological pH, temperature, etc.), or under conditions of an assay. In some embodiments, such a link can remain stable for at least 1 hour, at least 6 hours, at least 12 hours, at least 1 day, at least 1 week, at least 1 month, at least 1 year, or a range between any two foregoing values.

In some embodiments, the affinity between an antigen and antigen binding molecule can be not compromised by the conjugation of a reporter oligonucleotide to the antigen. In some such embodiments, the presence of the oligonucleotide or the process of conjugating the oligonucleotide to the antigen may not increase or decrease the affinity of the antigen to the antigen binding molecule.

In some embodiments, a reporter oligonucleotide can be conjugated to a labelling agent (e.g., antigen) directly using any suitable chemical moiety on the labelling agent (e.g., antigen). In some embodiments, a reporter oligonucleotide can be conjugated to an labelling agent (e.g., antigen) enzymatically, e.g., by ligation. In some cases, a reporter oligonucleotide can be linked indirectly to a labelling agent (e.g., antigen), for example via a non-covalent interaction such as a biotin/streptavidin interaction or an equivalent thereof, via an aptamer or secondary antibody, or via a protein-protein interaction such as a leucine-zipper tag interaction or the like.

In some embodiments, a reporter oligonucleotide can be conjugated to a labelling agent (e.g., antigen) using click chemistry, or a similar method. Click chemistry can refer to a class of biocompatible small molecule reactions that can facilitate the joining of molecules, such as a reporter oligonucleotide and a labelling agent (e.g., antigen). A click reaction can be a one pot reaction, and in some cases is not disturbed by water. A click reaction can generate minimal byproducts, non-harmful byproducts, or no byproducts. A click reaction can be driven by a large thermodynamic force. In some cases, a click reaction can be driven quickly and/or irreversibly to a high yield of a single reaction product (e.g., a reporter oligonucleotide conjugated to an antigen), and can have high reaction specificity. Click reactions can include but are not limited to [3+2] cycloadditions, thiol-ene reactions, Diels-Alder reactions, inverse electron demand Diels-Alder reactions, [4+1] cycloadditions, nucleophilic substitutions, carbonyl-chemistry-like formation of ureas, or addition reactions to carbon-carbon double bonds (e.g., dihydroxylation).

In some embodiments, a labelling agent (e.g., antigen) can be conjugated to a reporter oligonucleotide by a redox activated chemical tagging (ReACT) reaction. A react reaction can be a chemoselective methionine-bioconjugation that can employ redox reactivity. In some embodiments, for example, oxaziridine-based reagents can enable highly selective, rapid, and robust conjugation. Further description of ReACT chemistry can be found, for example, in (Makishma, Akio. Biochemistry for Materials Science. Elsevier, 2019).

In some embodiments, a labelling agent (e.g., antigen) can be conjugated to a reporter oligonucleotide by a site-specific sortase motif-dependent conjugation. Site-specific sortase motif-dependent conjugation can be a highly specific platform for conjugation that can rely on the specificity of Aortase A for short peptide sequences (e.g., LPXTG and GGG).

Sortase A can be a transpeptidase that can be adopted for site-specific protein modification. A reaction catalyzed by Sortase A can result in the formation of an amide bond between a C terminal sorting motif (e.g., LPXTG, where X can be any amino acid) and an N terminal oligoglycine. Such a conjugation reaction can proceed by first cleaving the peptide bond between the threonine and glycine residues with the sorting motif of Sortase A. Sortase A can be used to conjugate an oligonucleotide to either an N terminus or a C terminus of a labelling agent (e.g., antigen). Sortase A can retain its specificity while accepting a wide range of potential substrates.

In some embodiments, a labelling agent (e.g., antigen) can be conjugated to a reporter oligonucleotide by a site-specific photo-crosslinking-dependent conjugation. For example, such photo-crosslinking dependent conjugation can utilize unnatural amino acids or chemical crosslinking. Such photo-crosslinking can be mediated or directed by a peptide in some cases. For example, a peptide or other photosensitive molecule on the labelling agent (e.g., antigen) can form a covalent bond with a molecule on the oligonucleotide upon activation by a specified wavelength of light. In some embodiments, a peptide or other photosensitive molecule on the reporter oligonucleotide can form a covalent bond with a residue on the labelling agent (e.g., antigen) upon activation by a specified wavelength of light.

In some embodiments, an labelling agent (e.g., antigen) can be conjugated to a reporter oligonucleotide by site-specific conformation-dependent conjugation (e.g., glycan-dependent Fc conjugation or GlyCLICK). Such conjugation can generate a reporter oligonucleotide conjugated labelling agent (e.g., antigen). For example, deglycosylation of the labelling agent (e.g., antigen) can facilitate site specific conjugation using click chemistry techniques. In some embodiment, an labelling agent (e.g., antigen) can be conjugated to a reporter oligonucleotide by nitrilotriacetate conjugation

An oligonucleotide can be conjugated to a constant region of an antigen. For example, an oligonucleotide can be conjugated to a constant region of a heavy chain or a constant region of a light chain of an antigen that is an antibody or antigen binding fragment thereof.

An oligonucleotide can be conjugated to a variable region of an antigen. For example, an oligonucleotide can be conjugated to a variable region of a heavy chain or a variable region of a light chain of an antigen that is an antibody or antigen binding fragment thereof.

The reporter oligonucleotide conjugated labelling agent (e.g., antigen) can include one or more detectable tags. For example, in some instances, the reporter oligonucleotide conjugated labelling agent (e.g., antigen) can include a fluorophore, metal ion, or other detectable tag. The detectable tag can be conjugated to the reporter oligonucleotide, the antigen, or both.

A reporter oligonucleotide conjugated to a labelling agent (e.g., antigen) (e.g., a therapeutic antibody or antibody-drug conjugate) can include a sequence that identifies the labelling agent (e.g., antigen) (e.g., a reporter barcode sequence). In some instances, each labelling agent (e.g., antigen) can be conjugated to a reporter oligonucleotide containing a unique barcode sequence (e.g., a reporter barcode, such as a barcode sequence) that identifies the labelling agent (e.g., antigen) allowing different labelling agents (e.g., antigens) to be distinguished from one another, e.g., in a multiplexed antigen assay. In addition to the reporter barcode sequence, in some embodiments, the reporter oligonucleotide conjugated to the labelling agent (e.g., antigen) (e.g., a therapeutic antibody or antibody-drug conjugate) can include additional sequences that facilitate the processing and identification of the reporter barcode sequence (e.g., through nucleic acid sequencing of barcoded nucleic acid molecules). For example, the reporter oligonucleotide can contain one or more of: an adapter sequence, a primer or primer binding sequence, a sequencing primer or sequencing primer binding sequence (such as an R1 or partial R1 sequence), a unique molecular identifier (UMI), a polynucleotide sequence (such as a poly-A or poly-C sequence), or a sequence configured to bind to the flow cell of a sequencer (such as a P5 or P7, or partial sequences thereof).

### Antigen Binding Molecules

As used herein, the term “antigen binding molecule” refers to a molecule capable of binding an antigen. In some embodiments, an antigen binding molecule can be an antibody or antigen binding fragment thereof. In some embodiments, an antigen binding molecule can be an antibody (such as an ADA) or antigen binding fragment thereof produced by a subject. In some embodiments, the antibody or antigen binding fragment thereof can have affinity to an antigen provided herein. In some embodiments, the antibody or antigen binding fragment thereof can have affinity to an antibody or antibody-based drug, for example an antibody or antibody-based drug that can be administered to a subject. In some embodiments, the antigen binding molecule can have affinity to an antigen that is a biologic or a small molecule. For example, in some embodiments, the antigen binding molecule can have affinity to a component of a vaccine composition.

For example, in some embodiments, the antigen binding molecule can be antibody or antigen binding fragment thereof of which the epitope binding specificity is known, a.k.a., a known antibody. In other embodiments, the antigen binding molecule can be antibody or antigen binding fragment thereof of which the epitope binding specificity is to be determined, a.k.a., a candidate antibody. As used herein, the term “antibody” can refer to an immunoglobulin (Ig), polypeptide, or a protein having a binding domain which is, or is homologous to, an antigen binding domain. The term can further include “antigen binding fragments” and other interchangeable terms for similar binding fragments as described herein. In some embodiments, the antigen binding molecule comprises a known antibody. In other embodiments, the antigen binding molecule comprises a candidate antibody.

In some embodiments, an antigen or an antigen binding molecule (such as the known antibody and/or the candidate antibody provided herein) can be a therapeutic antibody or antigen binding fragment thereof (e.g., a monoclonal antibody). A therapeutic antibody or antigen binding fragment thereof can be a drug candidate or an FDA approved drug or therapeutic, such as a monoclonal antibody that is approved by the FDA for therapeutic use. Non-limiting examples of FDA approved monoclonal antibodies are provided in Table 1.

TABLE 1
FDA Approved Therapeutic Monoclonal Antibodies and other immunotherapies

| Antibody | Brand name | Type | Target |
|---|---|---|---|
| abciximab | ReoPro | chimeric Fab | GPIIb/IIIa |
| adalimumab | Humira | fully human | TNF |
| adalimumab-atto | Amjevita | fully | TNF |
| | | human, biosimilar | |
| ado-trastuzumab emtansine | Kadcyla | humanized, antibody- | HER2 |
| | | drug conjugate | |
| alemtuzumab | Campath, | humanized | CD52 |
| | Lemtrada | | |
| alirocumab | Praluent | fully human | PCSK9 |
| atezolizumab | Tecentriq | humanized | PD-L1 |
| atezolizumab | Tecentriq | humanized | PD-L1 |
| avelumab | Bavencio | fully human | PD-L1 |
| basiliximab | Simulect | chimeric | IL2RA |
| belimumab | Benlysta | fully human | BLyS |
| bevacizumab | Avastin | humanized | VEGF |
| bezlotoxumab | Zinplava | fully human | Clostridium |
| | | | difficile toxin B |
| blinatumomab | Blincyto | mouse, bispecific | CD19 |
| brentuximab vedotin | Adcetris | chimeric, antibody- | CD30 |
| | | drug conjugate | |
| brodalumab | Siliq | chimeric | IL 17RA |
| canakinumab | Ilaris | fully human | IL1B |
| capromab pendetide | ProstaScint | murine, radiolabeled | PSMA |
| certolizumab pegol | Cimzia | humanized | TNF |
| cetuximab | Erbitux | chimeric | EGFR |
| daclizumab | Zenapax | humanized | IL2RA |
| daclizumab | Zinbryta | humanized | IL2R |
| daratumumab | Darzalex | fully human | CD38 |
| denosumab | Prolia, Xgeva | fully human | RANKL |
| dinutuximab | Unituxin | chimeric | GD2 |
| dupilumab | Dupixent | fully human | IL4RA |
| durvalumab | Imfinzi | fully human | PD-L1 |
| eculizumab | Soliris | humanized | Complement |
| | | | component 5 |
| elotuzumab | Empliciti | humanized | SLAMF7 |
| evolocumab | Repatha | fully human | PCSK9 |
| golimumab | Simponi | fully human | TNF |
| golimumab | Simponi Aria | fully human | TNF |
| ibritumomab tiuxetan | Zevalin | murine, | CD20 |
| | | radioimmunotherapy | |
| idarucizumab | Praxbind | humanized Fab | dabigatran |
| infliximab | Remicade | chimeric | TNF alpha |
| infliximab-abda | Renflexis | chimeric, biosimilar | TNF |
| infliximab-dyyb | Inflectra | chimeric, biosimilar | TNF |
| ipilimumab | Yervoy | fully human | CTLA-4 |
| ixekizumab | Taltz | humanized | IL17A |
| mepolizumab | Nucala | humanized | IL5 |
| natalizumab | Tysabri | humanized | alpha-4 integrin |
| necitumumab | Portrazza | fully human | EGFR |
| nivolumab | Opdivo | fully human | PD-1 |
| nivolumab | Opdivo | fully human | PD-1 |
| obiltoxaximab | Anthem | chimeric | Protective |
| | | | antigen of |
| | | | the Anthrax |
| | | | toxin |
| obinutuzumab | Gazyva | humanized | CD20 |
| ocrelizumab | Ocrevus | humanized | CD20 |
| ofatumumab | Arzerra | fully human | CD20 |
| olaratumab | Lartruvo | fully human | PDGFRA |
| omalizumab | Xolair | humanized | IgE |
| palivizumab | Synagis | humanized | F protein |
| | | | of RSV |
| panitumumab | Vectibix | fully human | EGFR |
| pembrolizumab | Keytruda | humanized | PD-1 |
| pertuzumab | Perjeta | humanized | HER2 |
| ramucirumab | Cyramza | fully human | VEGFR2 |
| ranibizumab | Lucentis | humanized | VEGFR1, |
| | | | VEGFR2 |
| raxibacumab | Raxibacumab | fully human | Protective |
| | | | antigen |
| | | | of Bacillus |
| | | | anthracis |
| reslizumab | Cinqair | humanized | IL5 |
| rituximab | Rituxan | chimeric | CD20 |
| secukinumab | Cosentyx | fully human | IL17A |
| siltuximab | Sylvant | chimeric | IL6 |
| tocilizumab | Actemra | humanized | IL6R |
| tocilizumab | Actemra | humanized | IL6R |
| trastuzumab | Herceptin | humanized | HER2 |
| ustekinumab | Stelara | fully human | IL12 |
| ustekinumab | Stelara | fully human | IL12, IL23 |
| vedolizumab | Entyvio | humanized | integrin receptor |
| sarilumab | Kevzara | fully human | IL6R |
| rituximab and hyaluronidase | Rituxan | chimeric, co- | CD20 |
| | Hycela | formulated | |
| guselkumab | Tremfya | fully human | IL23 |
| inotuzumab ozogamicin | Besponsa | humanized, antibody- | CD22 |
| | | drug conjugate | |
| adalimumab-adbm | Cyltezo | fully | TNF |
| | | human, biosimilar | |
| gemtuzumab ozogamicin | Mylotarg | humanized, antibody- | CD33 |
| | | drug conjugate | |
| bevacizumab-awwb | Mvasi | humanized, biosimilar | VEGF |
| benralizumab | Fasenra | humanized | interleukin-5 |
| | | | receptor alpha |
| | | | subunit |
| emicizumab-kxwh | Hemlibra | humanized, bispecific | Factor |
| | | | IXa, Factor X |
| trastuzumab-dkst | Ogivri | humanized, biosimilar | HER2 |
| infliximab-qbtx | Ixifi | chimeric, biosimilar | TNF |
| ibalizumab-uiyk | Trogarzo | humanized | CD4 |
| tildrakizumab-asmn | Ilumya | humanized | IL23 |
| burosumab-twza | Crysvita | fully human | FGF23 |
| erenumab-aooe | Aimovig | fully human | CGRP receptor |

In some embodiments, an antigen or an antigen binding molecule (such as the known antibody and/or the candidate antibody provided herein) can be similar to an FDA approved therapeutic monoclonal antibody. In some such cases, an antigen or an antigen binding molecule (such as the known antibody and/or the candidate antibody provided herein) can have at least 75% identity, at least 80% identity, at least 85% identity, at least 90% identity, or at least 95% identity to an FDA approved therapeutic monoclonal antibody or a range between any two foregoing values. In some embodiments, an antigen or an antigen binding molecule (such as the known antibody and/or the candidate antibody provided herein) can have a heavy chain variable region that is at least 75% identity, at least 80% identity, at least 85% identity, at least 90% identity, or at least 95% identity to the heavy chain variable region of an FDA approved therapeutic monoclonal antibody or a range between any two foregoing values. In some embodiments, an antigen or an antigen binding molecule (such as the known antibody and/or the candidate antibody provided herein) can have a heavy chain constant region that is at least 75% identity, at least 80% identity, at least 85% identity, at least 90% identity, or at least 95% identity to the heavy chain constant region of an FDA approved therapeutic monoclonal antibody or a range between any two foregoing values. In some embodiments, an antigen or an antigen binding molecule (such as the known antibody and/or the candidate antibody provided herein) can have a light chain variable region that is at least 75% identity, at least 80% identity, at least 85% identity, at least 90% identity, or at least 95% identity to a light chain variable region of an FDA approved therapeutic monoclonal antibody or a range between any two foregoing values. In some embodiments, an antigen or an antigen binding molecule (such as the known antibody and/or the candidate antibody provided herein) can have a light chain constant region that is at least 75% identity, at least 80% identity, at least 85% identity, at least 90% identity, or at least 95% identity to the light chain constant region of an FDA approved therapeutic monoclonal antibody or a range between any two foregoing values. In some embodiments, an antigen or an antigen binding molecule (such as the known antibody and/or the candidate antibody provided herein) can be an antibody or antigen binding fragment thereof that has a target that is the same as the target of an FDA approved therapeutic monoclonal antibody.

In some embodiments, the antibody (such as the known antibody and/or the candidate antibody provided herein) is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody. In some specific embodiments, the antibody (such as the known antibody and/or the candidate antibody provided herein) is an oligoclonal antibody. The term “oligoclonal antibody” as used herein generally refers to a collection of antibodies that are derived from a few clones.

### Engineered Cells

In certain embodiments, the methods provided herein involve engineered cells expressing an antigen binding molecule (such as the known antibody and/or the candidate antibody provided herein). In some embodiments, a plurality of cells is engineered to express the same antibody, for example, the same known antibody or the same candidate antibody. In some embodiments, the plurality of cells expressing the same antibody exhibits an epitope specificity for an antigen. In some embodiments, the methods provided herein encompass a first plurality of cells engineered to express a known antibody. In some embodiments, the methods provided herein encompass a second plurality of cells engineered to express a candidate antibody. In some embodiments, the methods provided herein encompass multiple pluralities of cells, each engineered to express a different antibody. In some embodiments, the pluralities of cells are mixed together and contacted with an antigen.

Any cells that can be engineered to express antibodies are encompassed by the present disclosure. In some embodiments, the cells comprise immune cells (e.g., B cells, T cells, and the like). In certain embodiments, immune cells can be isolated from the blood or other biological samples of a subject, such as a human or other animal, that has been immunized or that is suffering from an infection, cancer, an autoimmune condition, or any other diseases to identify a pathogen-, tumor-, and/or disease specific antibody of potential clinical significance. Certain immune cells from immunized subjects make antibodies to one or more target antigens in question and/or one or more unknown antigens. In some embodiments, the cells comprise Ramos cells.

### Generating Single Cell Suspensions

In some embodiments, the methods further comprise generating a plurality of single cell suspensions. In some embodiments, the present disclosure encompasses the compartmentalization, depositing, or partitioning of one or more particles (e.g., cells (including the first plurality of cells, the second plurality of cells), the known antibody, the candidate antibody, beads, reagents, etc.) into discrete compartments or partitions (referred to interchangeably herein as partitions), where each partition maintains separation of its own contents from the contents of other partitions.

The term “partition,” as used herein, generally, refers to a space or volume that can be suitable to contain one or more species or conduct one or more reactions. A partition can be a physical compartment, such as a droplet or well. The partition can isolate space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

A partition can be a droplet in an emulsion. A partition can include one or more other partitions. A partition can include one or more particles. A partition can include one or more types of particles. For example, a partition of the present disclosure can include one or more biological particles and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition may not include a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, can be injected into the droplets previous to, subsequent to, or concurrently with droplet generation, such as via a support (e.g., bead), as described elsewhere herein.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In some instances, a droplet can be formed by creating an emulsion by mixing or agitating immiscible phases. Mixing or agitation can include various agitation techniques, such as vortexing, pipetting, tube flicking, or other agitation techniques. In some cases, mixing or agitation can be performed without using a microfluidic device. In some examples, a droplet can be formed by exposing a mixture to ultrasound or sonication. For example, to partition contents into droplets, a mixture including a first fluid, a second fluid, optionally a surfactant, and the contents can be subject to such agitation techniques to generate a plurality of droplets (first fluid-in-second fluid or second fluid-in-first fluid) including the contents, or subsets thereof. In an example, a mixture includes beads. Upon agitation, the beads in the mixture can limit droplet break-up into droplets smaller than the size of the beads, and a substantially monodisperse population of droplets comprising the beads can result.

In the case of droplets in an emulsion, allocating individual particles to discrete partitions can, in one non-limiting example, be accomplished by introducing a flowing stream of particles in an aqueous fluid into a flowing stream or reservoir of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions can contain at most one biological particle (e.g., bead, DNA, cell or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

### Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

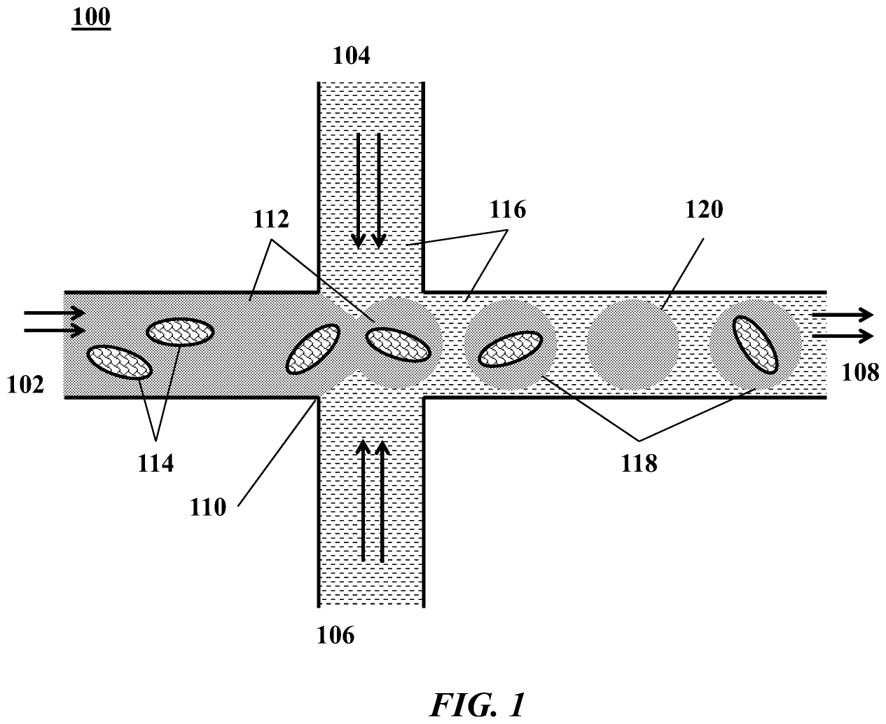

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle 114 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can contain an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, and (2) unoccupied droplets 120, not containing any biological particles 114. Occupied droplets 118 can contain singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions can include more than one biological particle. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles 114) at the partitioning junction 110, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

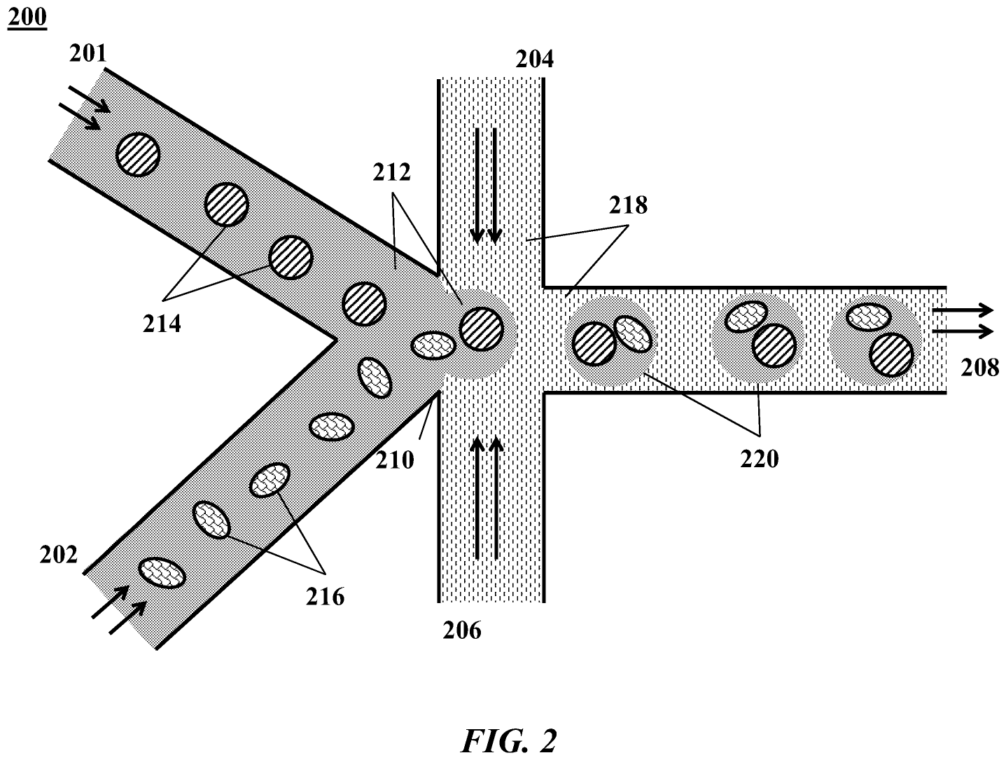

As will be appreciated, the above-described occupancy rates are also applicable to partitions that include both biological particles and additional reagents, including, but not limited to, supports, such as beads (e.g., gel beads) carrying partition-specific barcode molecules (described in relation to FIG. 2). The occupied partitions (e.g., at least about 10%, 20%, 30%, 40%, 50%, 60%, 70%, 80%, 90%, 95%, or 99% of the occupied partitions) can include both a support (e.g., a bead) comprising partition-specific barcode molecules and a biological particle.

In another aspect, in addition to or as an alternative to droplet-based partitioning, biological particles can be encapsulated within a porous matrix in which is entrained one or more individual biological particles or small groups of biological particles. Preparation of supports (e.g., beads) comprising biological particles, e.g., cells, can be performed by a variety of methods. For example, air knife droplet or aerosol generators can be used to dispense droplets of precursor fluids into gelling solutions in order to form beads (e.g., gel beads) that include individual biological particles or small groups of biological particles. Likewise, membrane-based encapsulation systems can be used to generate beads comprising encapsulated biological particles as described herein. Microfluidic systems of the present disclosure, such as that shown in FIG. 1, can be readily used in encapsulating biological particles (e.g., cells) as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 can also include an initiator (not shown) to cause polymerization and/or crosslinking of the polymer precursor to form the porous matrix that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the support, such as through passage of time or upon application of a particular stimulus, that degrades the support sufficiently to allow the biological particles (e.g., cell), or its other contents to be released from the support, such as into a partition (e.g., droplet). See, for example, U.S. Patent Application Publication No. 2014/0378345, which is entirely incorporated herein by reference for all purposes.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 can transport an aqueous fluid 212 that includes a plurality of beads 214 along the channel segment 201 into junction 210. The plurality of beads 214 can be sourced from a suspension of beads. For example, the channel segment 201 can be connected to a reservoir comprising an aqueous suspension of beads 214. The channel segment 202 can transport the aqueous fluid 212 that includes a plurality of biological particles 216 along the channel segment 202 into junction 210. The plurality of biological particles 216 can be sourced from a suspension of biological particles. For example, the channel segment 202 can be connected to a reservoir comprising an aqueous suspension of biological particles 216. In some instances, the aqueous fluid 212 in either the first channel segment 201 or the second channel segment 202, or in both segments, can include one or more reagents, as further described below.

A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 can deliver the discrete droplets to an outlet reservoir fluidly coupled to the channel segment 208, where they may be harvested. The second fluid 218 can contain an oil, such as a fluorinated oil, that includes a fluorosurfactant for stabilizing the resulting droplets, for example, inhibiting subsequent coalescence of the resulting droplets 220.

A discrete droplet that is generated can include an individual biological particle 220. A discrete droplet that is generated can include barcodes (e.g., a reporter barcode, such as a barcode sequence, and a partition-specific barcode) or other reagent carrying bead 214. A discrete droplet generated can include both an individual biological particle and a barcode carrying bead, such as droplets 220. In some instances, a discrete droplet can include more than one individual biological particle or no biological particle. In some instances, a discrete droplet can include more than one bead or no bead. A discrete droplet can be unoccupied (e.g., no beads, no biological particles).

Beneficially, a discrete droplet partitioning a biological particle and a barcode carrying bead can effectively facilitate the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 (FIG. 2) may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

As an alternative, the channel segments 201 and 202 can meet at another junction upstream of the junction 210. At such junction, beads and biological particles can form a mixture that is directed along another channel to the junction 210 to yield droplets 220. The mixture can provide the beads and biological particles in an alternating fashion, such that, for example, a droplet includes a single bead and a single biological particle.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments of the microfluidic devices described elsewhere herein can be coupled to any of a variety of different fluid sources or receiving components, including reservoirs, tubing, manifolds, or fluidic components of other systems. As will be appreciated, the microfluidic channel structures can have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

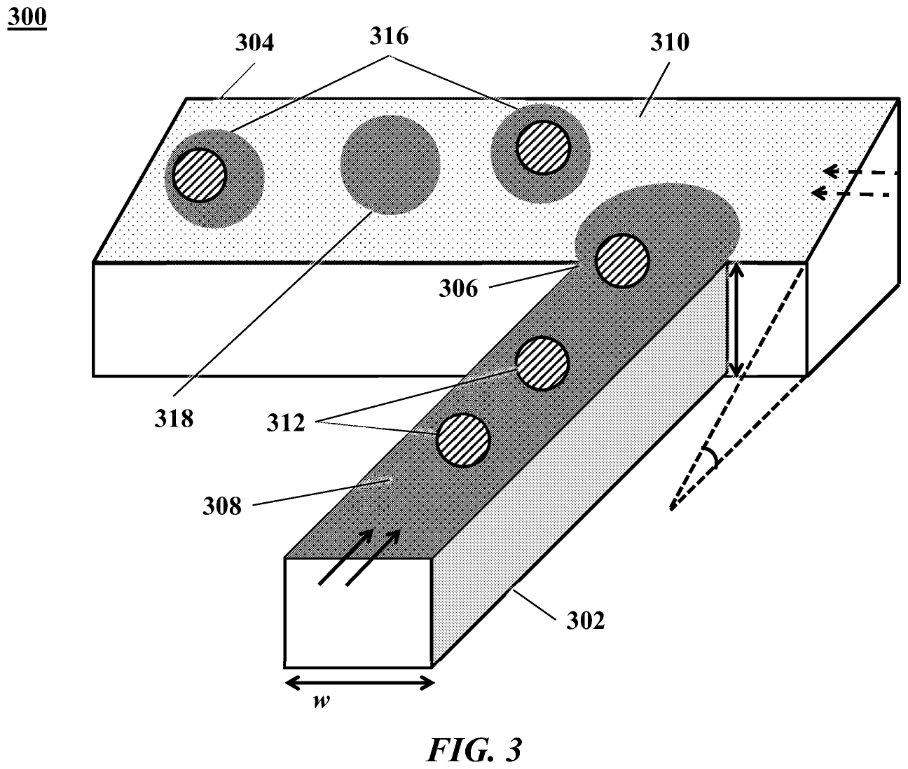

FIG. 3 shows an example of a microfluidic channel structure for the controlled partitioning of beads into discrete droplets. A channel structure 300 can include a channel segment 302 communicating at a channel junction 306 (or intersection) with a reservoir 304. The reservoir 304 can be a chamber. Any reference to “reservoir,” as used herein, can also refer to a “chamber.” In operation, an aqueous fluid 308 that includes suspended beads 312 can be transported along the channel segment 302 into the junction 306 to meet a second fluid 310 that is immiscible with the aqueous fluid 308 in the reservoir 304 to create droplets 316, 318 of the aqueous fluid 308 flowing into the reservoir 304. At the junction 306 where the aqueous fluid 308 and the second fluid 310 meet, droplets can form based on factors such as the hydrodynamic forces at the junction 306, flow rates of the two fluids 308, 310, fluid properties, and certain geometric parameters (e.g., w, ho, a, etc.) of the channel structure 300. A plurality of droplets can be collected in the reservoir 304 by continuously injecting the aqueous fluid 308 from the channel segment 302 through the junction 306.

A discrete droplet generated can include a bead (e.g., as in occupied droplets 316). Alternatively, a discrete droplet generated can include more than one bead. Alternatively, a discrete droplet generated may not include any beads (e.g., as in unoccupied droplet 318). In some instances, a discrete droplet generated can contain one or more biological particles, as described elsewhere herein. In some instances, a discrete droplet generated can contain one or more reagents, as described elsewhere herein.

In some instances, the aqueous fluid 308 can have a substantially uniform concentration or frequency of beads 312. The beads 312 can be introduced into the channel segment 302 from a separate channel (not shown in FIG. 3). The frequency of beads 312 in the channel segment 302 may be controlled by controlling the frequency in which the beads 312 are introduced into the channel segment 302 and/or the relative flow rates of the fluids in the channel segment 302 and the separate channel. In some instances, the beads can be introduced into the channel segment 302 from a plurality of different channels, and the frequency controlled accordingly.

In some instances, the aqueous fluid 308 in the channel segment 302 can contain biological particles (e.g., described with reference to FIGS. 1 and 2). In some instances, the aqueous fluid 308 can have a substantially uniform concentration or frequency of biological particles. As with the beads, the biological particles can be introduced into the channel segment 302 from a separate channel. The frequency or concentration of the biological particles in the aqueous fluid 308 in the channel segment 302 can be controlled by controlling the frequency in which the biological particles are introduced into the channel segment 302 and/or the relative flow rates of the fluids in the channel segment 302 and the separate channel. In some instances, the biological particles can be introduced into the channel segment 302 from a plurality of different channels, and the frequency controlled accordingly. In some instances, a first separate channel can introduce beads and a second separate channel can introduce biological particles into the channel segment 302. The first separate channel introducing the beads can be upstream or downstream of the second separate channel introducing the biological particles.

The second fluid 310 can include an oil, such as a fluorinated oil, that includes a fluorosurfactant for stabilizing the resulting droplets, for example, inhibiting subsequent coalescence of the resulting droplets.

In some instances, the second fluid 310 may not be subjected to and/or directed to any flow in or out of the reservoir 304. For example, the second fluid 310 can be substantially stationary in the reservoir 304. In some instances, the second fluid 310 can be subjected to flow within the reservoir 304, but not in or out of the reservoir 304, such as via application of pressure to the reservoir 304 and/or as affected by the incoming flow of the aqueous fluid 308 at the junction 306. Alternatively, the second fluid 310 can be subjected and/or directed to flow in or out of the reservoir 304. For example, the reservoir 304 can be a channel directing the second fluid 310 from upstream to downstream, transporting the generated droplets.

The channel structure 300 at or near the junction 306 can have certain geometric features that at least partly determine the sizes of the droplets formed by the channel structure 300. The channel segment 302 can have a height, ho and width, w, at or near the junction 306. By way of example, the channel segment 302 can include a rectangular cross-section that leads to a reservoir 304 having a wider cross-section (such as in width or diameter). Alternatively, the cross-section of the channel segment 302 can be other shapes, such as a circular shape, trapezoidal shape, polygonal shape, or any other shapes. The top and bottom walls of the reservoir 304 at or near the junction 306 can be inclined at an expansion angle, a. The expansion angle, a, allows the tongue (portion of the aqueous fluid 308 leaving channel segment 302 at junction 306 and entering the reservoir 304 before droplet formation) to increase in depth and facilitate decrease in curvature of the intermediately formed droplet. Droplet size can decrease with increasing expansion angle. The resulting droplet radius, Rd, can be predicted by the following equation for the aforementioned geometric parameters of h_{θ}, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 1°, 2°, 3°, 4°, 5°, 6°, 7°, 8°, 9°, 10°, 15°, 20°, 25°, 30°, 35°, 40°, 45°, 50°, 55°, 60°, 65°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 308 entering the junction 306 can be between about 0.04 microliters (μL)/minute (min) and about 40 μL/min. In some instances, the flow rate of the aqueous fluid 308 entering the junction 306 can be between about 0.01 microliters (μL)/minute (min) and about 100 μL/min. Alternatively, the flow rate of the aqueous fluid 308 entering the junction 306 can be less than about 0.01 μL/min. Alternatively, the flow rate of the aqueous fluid 308 entering the junction 306 can be greater than about 40 μL/min, such as 45 μL/min, 50 μL/min, 55 μL/min, μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 308 entering the junction 306.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 306) between aqueous fluid 308 channel segments (e.g., channel segment 302) and the reservoir 304. Alternatively or in addition, the throughput of droplet generation can be increased by increasing the flow rate of the aqueous fluid 308 in the channel segment 302.

### Beads and Partition-Specific Barcode Molecules

Nucleic acid barcode molecules (e.g., partition-specific barcode molecules) can be delivered to a partition (e.g., a droplet or well) via a solid support or carrier (e.g., a bead). In some cases, nucleic acid barcode molecules (e.g., partition-specific barcode molecule) are initially associated with the solid support and then released from the solid support upon application of a stimulus, which allows the nucleic acid barcode molecules (e.g., partition-specific barcode molecule) to dissociate or to be released from the solid support. In specific examples, nucleic acid barcode molecules (e.g., partition-specific barcode molecule) are initially associated with the solid support (e.g., bead) and then released from the solid support upon application of a biological stimulus, a chemical stimulus, a thermal stimulus, an electrical stimulus, a magnetic stimulus, and/or a photo stimulus.

A nucleic acid barcode molecule (e.g., partition-specific barcode molecule) can contain a partition-specific barcode sequence and a functional sequence, such as a nucleic acid primer sequence or a template switch oligonucleotide (TSO) sequence.

The solid support can be a bead. A solid support, e.g., a bead, can be porous, non-porous, hollow, solid, semi-solid, and/or a combination thereof. Beads can be solid, semi-solid, semi-fluidic, fluidic, and/or a combination thereof. In some instances, a solid support, e.g., a bead, can be dissolvable, disruptable, and/or degradable. In some cases, a solid support, e.g., a bead, may not be degradable. In some cases, the solid support, e.g., a bead, can be a gel bead. A gel bead can be a hydrogel bead. A gel bead can be formed from molecular precursors, such as a polymeric or monomeric species. A semi-solid support, e.g., a bead, can be a liposomal bead. Solid supports, e.g., beads, can include metals including iron oxide, gold, and silver. In some cases, the solid support, e.g., the bead, can be a silica bead. In some cases, the solid support, e.g., a bead, can be rigid. In other cases, the solid support, e.g., a bead, can be flexible and/or compressible.

The term “bead,” as used herein, generally refers to a particle. The bead can be a solid or semi-solid particle. The bead can be a gel bead. The gel bead can include a polymer matrix (e.g., matrix formed by polymerization or cross-linking). The polymer matrix can include one or more polymers (e.g., polymers having different functional groups or repeat units). Polymers in the polymer matrix can be randomly arranged, such as in random copolymers, and/or have ordered structures, such as in block copolymers. Cross-linking can be via covalent, ionic, or inductive, interactions, or physical entanglement. The bead can be a macromolecule. The bead can be formed of nucleic acid molecules (e.g., partition-specific barcode molecules) bound together. The bead can be formed via covalent or non-covalent assembly of molecules (e.g., macromolecules), such as monomers or polymers. Such polymers or monomers can be natural or synthetic. Such polymers or monomers can be or include, for example, nucleic acid molecules (e.g., DNA or RNA). The bead can be formed of a polymeric material. The bead can be magnetic or non-magnetic. The bead can be rigid. The bead can be flexible and/or compressible. The bead can be disruptable or dissolvable. The bead can be a solid particle (e.g., a metal-based particle including but not limited to iron oxide, gold or silver) covered with a coating comprising one or more polymers. Such coating can be disruptable or dissolvable.

A partition can contain one or more unique identifiers, such as partition-specific barcodes. Partition-specific barcodes can be previously, subsequently or concurrently delivered to the partitions that hold the compartmentalized or partitioned biological particle. For example, partition-specific barcodes can be injected into droplets previous to, subsequent to, or concurrently with droplet generation. The delivery of the partition-specific barcodes to a particular partition allows for the later attribution of the characteristics of the individual biological particle to the particular partition. Partition-specific barcodes can be delivered, for example on a nucleic acid molecule (e.g., a partition-specific barcode molecule), to a partition via any suitable mechanism. Nucleic acid molecules (e.g., partition-specific barcode molecules) can be delivered to a partition via a support (e.g., a bead). A support, in some instances, can include a bead. Beads are described in further detail below.

In some cases, nucleic acid molecules (e.g., partition-specific barcode molecules) can be initially associated with the support and then released from the support. Release of the nucleic acid molecules (e.g., partition-specific barcode molecules) can be passive (e.g., by diffusion out of the support). In addition or alternatively, release from the support can be upon application of a stimulus which allows the nucleic acid nucleic acid molecules (e.g., partition-specific barcode molecules) to dissociate or to be released from the support. Such stimulus can disrupt the support, an interaction that couples the nucleic acid molecules (e.g., partition-specific barcode molecules) to or within the support, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486, each of which is herein entirely incorporated by reference for all purposes.

In some examples, beads, biological particles and droplets may flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988, which is entirely incorporated herein by reference.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μtm.

In certain aspects, beads can be provided as a population or plurality of beads having a relatively monodisperse size distribution. Where it can be desirable to provide relatively consistent amounts of reagents within partitions, maintaining relatively consistent bead characteristics, such as size, can contribute to the overall consistency. In particular, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can contain natural and/or synthetic materials. For example, a bead can contain a natural polymer, a synthetic polymer or both natural and synthetic polymers. Examples of natural polymers can include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some cases, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some cases, the bead can contain individual polymers that may be further polymerized together. In some cases, beads can be generated via polymerization of different precursors, such that they include mixed polymers, co-polymers, and/or block co-polymers. In some cases, the bead can contain covalent or ionic bonds between polymeric precursors (e.g., monomers, oligomers, linear polymers), nucleic acid molecules (e.g., partition-specific molecules), primers, and other entities. In some cases, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some cases, reversible cross-linking can also allow for reversible attachment of a material bound to the surface of a bead. In some cases, a cross-linker can form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., partition-specific molecules). Cystamine (including modified cystamines), for example, is an organic agent comprising a disulfide bond that can be used as a crosslinker agent between individual monomeric or polymeric precursors of a bead. Polyacrylamide can be polymerized in the presence of cystamine or a species comprising cystamine (e.g., a modified cystamine) to generate polyacrylamide gel beads containing disulfide linkages (e.g., chemically degradable beads comprising chemically-reducible cross-linkers). The disulfide linkages can permit the bead to be degraded (or dissolved) upon exposure of the bead to a reducing agent.

In some cases, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some cases, a bead can include an acrydite moiety, which in certain aspects can be used to attach one or more nucleic acid molecules (e.g., partition-specific barcode molecules) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., a partition-specific barcode molecule). Acrydite moieties can be modified with thiol groups capable of forming a disulfide bond or can be modified with groups already containing a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can contain a reactive hydroxyl group that can be used for attachment.

Functionalization of beads for attachment of nucleic acid molecules (e.g., partition-specific barcode molecules) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can contain acrydite moieties, such that when a bead is generated, the bead also contain acrydite moieties. The acrydite moieties can be attached to a nucleic acid molecule (e.g., a partition-specific barcode molecule) that includes one or more functional sequences, such as a UMI sequence, a TSO sequence or a primer sequence (e.g., a poly T sequence, or a nucleic acid primer sequence complementary to a target nucleic acid sequence and/or for amplifying a target nucleic acid sequence, a random primer, or a primer sequence for messenger RNA) that is useful for incorporation into the bead, etc.) and/or one or more partition-specific barcode sequences. The one or more partition-specific barcode sequences can include sequences that are the same for all nucleic acid molecules (e.g., partition-specific barcode molecules) coupled to a given bead and/or sequences that are different across all nucleic acid molecules (e.g., partition-specific barcode molecules) coupled to the given bead. The nucleic acid molecule (e.g., partition-specific barcode molecules) can be incorporated into the bead.

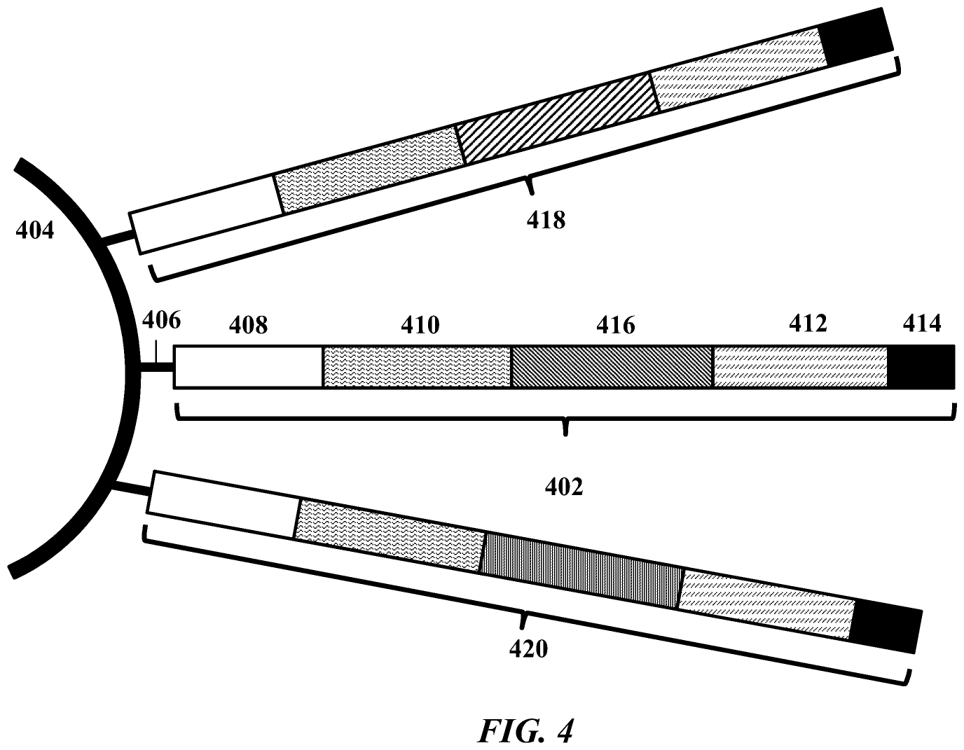

FIG. 4 illustrates an example of a bead carrying partition-specific barcode molecules. A nucleic acid molecule (e.g., a partition-specific barcode molecule) 402 can be coupled to a bead 404 by a releasable linkage 406, such as, for example, a disulfide linker. The same bead 404 can be coupled (e.g., via releasable linkage) to one or more other partition-specific barcode molecules 418, 420. The partition-specific barcode molecule 402 can contain a partition-specific barcode. As noted elsewhere herein, the structure of the partition-specific barcode can contain a number of sequence elements. The partition-specific barcode molecule 402 can contain a functional sequence 408 that can be used in subsequent processing. For example, the functional sequence 408 can include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The partition-specific barcode molecule 402 can contain a partition-specific barcode sequence 410 for use in barcoding the sample (e.g., DNA, RNA, protein, etc.). In some cases, the partition-specific barcode sequence 410 can be bead-specific such that the partition-specific barcode sequence 410 is common to all partition-specific barcode molecules (e.g., including partition-specific barcode molecule 402) coupled to the same bead 404. Alternatively or in addition, the partition-specific barcode sequence 410 can be partition-specific such that the partition-specific barcode sequence 410 is common to all nucleic acid molecules (e.g., partition-specific barcode molecules) coupled to one or more beads that are partitioned into the same partition. The partition-specific barcode molecule 402 can contain a specific priming sequence 412, such as an mRNA specific priming sequence (e.g., poly-T sequence), a targeted priming sequence, and/or a random priming sequence. The partition-specific barcode molecule 402 can contain an anchoring sequence 414 to ensure that sequence 412 hybridizes at the sequence end (e.g., of the mRNA). For example, the anchoring sequence 414 can include a random short sequence of nucleotides, such as a 1-mer, 2-mer, 3-mer or longer sequence, which can ensure that a poly-T segment is more likely to hybridize at the sequence end of the poly-A tail of the mRNA.

The partition-specific barcode molecule 402 can contain a unique molecular identifier (UMI) 416. In some cases, the unique molecular identifier (UMI) sequence 416 can include from about 5 to about 8 nucleotides. Alternatively, the unique molecular identifier (UMI) sequence 416 can include less than about 5 or more than about 8 nucleotides. The unique molecular identifier (UMI) sequence 416 can be a unique sequence that varies across individual partition-specific barcode molecules (e.g., 402, 418, 420, etc.) coupled to a single bead (e.g., bead 404). In some cases, the unique molecular identifier (UMI) sequence 416 can be a random sequence (e.g., such as a random N-mer sequence). For example, the UMI can provide a unique identifier of the starting mRNA molecule that was captured, in order to facilitate quantitation of the number of original expressed RNA. As will be appreciated, although FIG. 4 shows three partition-specific molecules 402, 418, 420 coupled to the surface of the bead 404, an individual bead can be coupled to any number of partition-specific barcode molecules, for example, from one to tens to hundreds of thousands or even millions of partition-specific barcode molecules. The respective barcodes for the individual partition-specific barcode molecule can contain both common sequence segments or relatively common sequence segments (e.g., 408, 410, 412, etc.) and variable or unique sequence segments (e.g., 416) between different individual partition-specific molecules coupled to the same bead.

In operation, a biological particle (e.g., cell, DNA, RNA, etc.) can be co-partitioned along with a barcode bearing bead 404. The partition-specific barcode molecules 402, 418, 420 can be released from the bead 404 in the partition. By way of example, in the context of analyzing sample RNA, the poly-T segment (e.g., 412) of one of the released partition-specific molecules (e.g., 402) can hybridize to the poly-A tail of a mRNA molecule. Reverse transcription can result in a cDNA transcript of the mRNA, but which transcript includes each of the sequence segments 408, 410, 416 of the nucleic acid molecule 402. Because the partition-specific barcode molecule 402 can include an anchoring sequence 414, it will more likely hybridize to and prime reverse transcription at the sequence end of the poly-A tail of the mRNA. Within any given partition, all of the cDNA transcripts of the individual mRNA molecules can include a common barcode sequence segment 410. However, the transcripts made from the different mRNA molecules within a given partition can vary at the unique molecular identifying sequence 412 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the partition-specific barcode molecules into the partition, in some cases, the partition-specific barcode molecule partition-specific barcode molecules bound to the bead (e.g., gel bead) can be used to hybridize and capture the mRNA on the solid phase of the bead, for example, in order to facilitate the separation of the RNA from other cell contents.

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions may be collected, pooled, and subjected to further processing (e.g., reverse transcription, adapter attachment, amplification, clean up, sequencing). In other instances, one or more of the processing methods, e.g., reverse transcription, may occur in the partition. For example, conditions sufficient for barcoding, adapter attachment, reverse transcription, or other nucleic acid processing operations may be provided in the partition and performed prior to clean up and sequencing.

In some instances, a partition-specific barcode molecule can contain a capture sequence configured to bind to a corresponding capture handle sequence. In some embodiments, a capture sequence includes a template switching oligonucleotide (TSO) sequence. In some instances, a bead can be conjugated to a plurality of partition-specific barcode molecules containing different capture sequences configured to bind to different respective corresponding capture handle sequences. For example, a partition-specific barcode molecule can contain a first subset of one or more capture sequences each configured to bind to a first corresponding capture handle sequence, a second subset of one or more capture sequences each configured to bind to a second corresponding capture handle sequence, a third subset of one or more capture sequences each configured to bind to a third corresponding capture handle sequence, and etc. A bead can be conjugated to any number of different partition-specific barcode molecules containing any number of different capture sequences. In some instances, a bead can contain at least 2, 3, 4, 5, 6, 7, 8, 9, 10 or more different capture sequences configured to bind to different respective capture handle sequences, respectively. Alternatively or in addition, a bead can contain at most about 10, 9, 8, 7, 6, 5, 4, 3, or 2 different capture sequences configured to bind to different respective capture handle sequences. In some instances, the different capture sequences can be configured to facilitate analysis of a same type of analyte. In some instances, the different capture sequences can be configured to facilitate analysis of different types of analytes (with the same bead). The capture sequence can be designed to attach to a corresponding capture handle sequence. Beneficially, such corresponding capture sequence can be introduced to, or otherwise induced in, a biological particle (e.g., cell, cell bead, etc.) for performing different assays in various formats (e.g., barcoded antibodies containing the corresponding capture handle sequence, barcoded MHC dextramers containing the corresponding capture handle sequence, barcoded guide RNA molecules containing the corresponding capture handle sequence, etc.), such that the corresponding capture handle sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

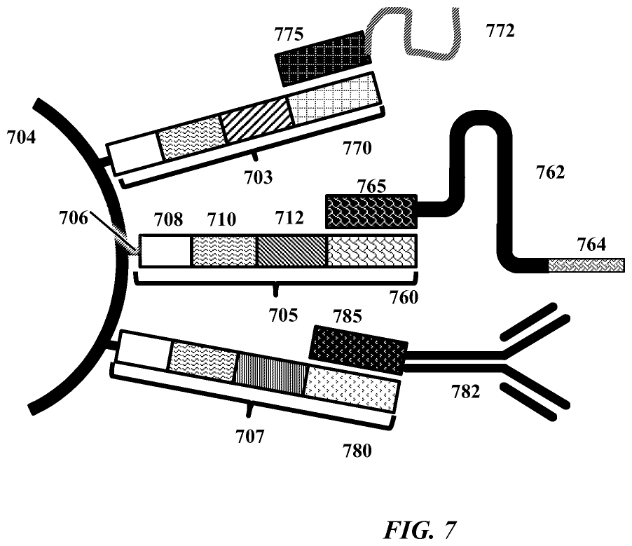

FIG. 7 illustrates a non-limiting example of a barcode carrying bead in accordance with some embodiments of the disclosure. A nucleic acid molecule 705, such as an oligonucleotide, can be coupled to a bead 707 by a releasable linkage 706, such as, for example, a disulfide linker. The nucleic acid molecule 705 can include a first capture sequence 760. The same bead 704 can be coupled, e.g., via releasable linkage, to one or more other nucleic acid molecules 703, 707 including other capture sequences. The nucleic acid molecule 705 can be or include a barcode. As described elsewhere herein, the structure of the barcode can include a number of sequence elements, such as a functional sequence 708 (e.g., flow cell attachment sequence, sequencing primer sequence, etc.), a barcode sequence 710 (e.g., bead-specific sequence common to bead, partition-specific sequence common to partition, etc.), and a unique molecular identifier 712 (e.g., unique sequence within different molecules attached to the bead), or partial sequences thereof. The capture sequence 760 can be configured to attach to a corresponding capture sequence 765 (e.g., capture handle). In some instances, the corresponding capture sequence 765 can be coupled to another molecule that can be an analyte or an intermediary carrier. For example, as illustrated in FIG. 7, the corresponding capture sequence 765 is coupled to a guide RNA molecule 762 including a target sequence 764, wherein the target sequence 764 is configured to attach to the analyte. Another oligonucleotide molecule 707 attached to the bead 704 includes a second capture sequence 780 which is configured to attach to a second corresponding capture sequence (e.g., capture handle) 785. As illustrated in FIG. 7, the second corresponding capture sequence 785 is coupled to an antibody 782. In some cases, the antibody 782 can have binding specificity to an analyte (e.g., surface protein). Alternatively, the antibody 782 cannot have binding specificity. Another oligonucleotide molecule 703 attached to the bead 704 includes a third capture sequence 770 which is configured to attach to a third corresponding capture sequence 775. As illustrated in FIG. 7, the third corresponding capture sequence (e.g., capture handle) 775 is coupled to a molecule 772. The molecule 772 may or may not be configured to target an analyte. The other oligonucleotide molecules 703, 707 can include the other sequences (e.g., functional sequence, barcode sequence, UMI, etc.) described with respect to oligonucleotide molecule 705. While a single oligonucleotide molecule including each capture sequence is illustrated in FIG. 7, it will be appreciated that, for each capture sequence, the bead can include a set of one or more oligonucleotide molecules each including the capture sequence. For example, the bead can include any number of sets of one or more different capture sequences. Alternatively or in addition, the bead 704 can include other capture sequences. Alternatively or in addition, the bead 704 can include fewer types of capture sequences (e.g., two capture sequences). Alternatively or in addition, the bead 704 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

In operation, the partition-specific barcode molecules can be released (e.g., in a partition), as described elsewhere herein. Alternatively, the nucleic acid molecules (e.g., partition-specific barcode molecules) bound to the bead (e.g., gel bead) can be used to hybridize and capture analytes (e.g., one or more types of analytes) on the solid phase of the bead.

In some cases, precursors containing a functional group that is reactive or capable of being activated such that it becomes reactive can be polymerized with other precursors to generate gel beads containing the activated or activatable functional group. The functional group can then be used to attach additional species (e.g., disulfide linkers, primers, other oligonucleotides, etc.) to the gel beads. For example, some precursors containing a carboxylic acid (COOH) group can co-polymerize with other precursors to form a gel bead that also includes a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network can be functionalized with additional species via reduction of some of the disulfide linkages to free thiols (see e.g., U.S. patent Ser. No. 10/323,279, which is incorporated herein by reference in its entirety).

A bead injected or otherwise introduced into a partition may include releasably, cleavably, or reversibly attached barcodes. A bead injected or otherwise introduced into a partition may include activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as nucleic acid barcode molecules (e.g., partition-specific barcode molecules), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a partition-specific barcode molecule) can result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead can be introduced into a partition, such as a droplet of an emulsion or a well, such that the bead degrades within the partition and any associated species (e.g., partition-specific barcode molecules) are released within the droplet when the appropriate stimulus is applied. The free species may interact with other reagents contained in the partition. For example, a polyacrylamide bead comprising cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., partition-specific barcode molecules) can be associated with a bead such that, upon release from the bead, the molecular tag molecules (e.g., partition-specific barcode molecules) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., partition-specific barcode molecule) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties comprising a labile bond are incorporated into a bead, the bead may also include the labile bond. The labile bond may be, for example, useful in reversibly linking (e.g., covalently linking) species (e.g., a partition-specific barcode molecule) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the support (e.g., a bead, such as a gel bead).

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., partition-specific barcode molecules) that are attached to a solid support (e.g., a bead) may include a U-excising element that allows the species to release from the bead. In some cases, the U-excising element may include a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads include the species upon bead formation. In some cases, such species may be added to the gel beads after formation. Such species may include, for example, nucleic acid molecules (e.g., partition-specific molecules), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may include one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may include a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a partition-specific barcode molecule) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that include degradable chemical crosslinkers, such as BAC or cystamine. Degradation of such degradable crosslinkers may be accomplished through a number of mechanisms (see e.g., U.S. patent Ser. No. 10/323,279, which is incorporated herein by reference in its entirety).

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Although FIGS. 1-3 have been described in terms of providing substantially singly occupied partitions, above, in certain cases, it can be desirable to provide multiply occupied partitions, e.g., containing two, three, four or more cells and/or supports (e.g., beads) comprising partition-specific barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional supports (e.g., beads) can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of supports (e.g., beads) from each source, while ensuring a given pairing or combination of such beads into a partition with a given number of biological particles (e.g., one biological particle and one bead per partition).

The partitions described herein may include small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with supports, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may include both unoccupied partitions (e.g., empty partitions) and occupied partitions.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, etc.) while the adjacent microwell can be used to contain a support (e.g., a bead such as a gel bead), droplet, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a support (e.g., a bead such as a gel bead) that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the support or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety

A well can include free reagents and/or reagents encapsulated in, or otherwise coupled to or associated with, supports (e.g., beads), or droplets. In some embodiments, any of the reagents described in this disclosure can be encapsulated in, or otherwise coupled to, a support (e.g., a bead) or a droplet, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a support (e.g., a bead) or droplet that includes a set of reagents that has a similar attribute, for example, a set of enzymes, a set of minerals, a set of oligonucleotides, a mixture of different barcode molecules, or a mixture of identical barcode molecules. In other cases, a support (e.g., a bead) or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different support (e.g., a bead) or droplet, or within a solution within a partition (e.g., microwell) of the system.

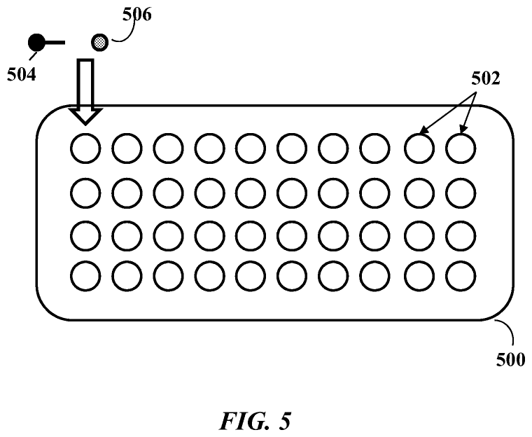

A non-limiting example of a microwell array in accordance with some embodiments of the disclosure is schematically presented in FIG. 5. In this example, the array can be contained within a substrate 500. The substrate 500 includes a plurality of wells 502. The wells 502 can be of any size or shape, and the spacing between the wells, the number of wells per substrate, as well as the density of the wells on the substrate 500 can be modified, depending on the particular application. In one such example application, a sample molecule 506, which can include a cell or cellular components (e.g., nucleic acid molecules) is co-partitioned with a bead 504, which can include a nucleic acid barcode molecule coupled thereto. The wells 502 can be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents can be loaded into a well either sequentially or concurrently. In some cases, reagents are introduced to the device either before or after a particular operation. In some cases, reagents (which can be provided, in certain instances, in supports (e.g., beads) or droplets) are introduced sequentially such that different reactions or operations occur at different steps. The reagents (or supports (e.g., beads) or droplets) can also be loaded at operations interspersed with a reaction or operation step. For example, supports (e.g., beads) (or droplets) including reagents for fragmenting polynucleotides (e.g., restriction enzymes) and/or other enzymes (e.g., transposases, ligases, polymerases, etc.) can be loaded into the well or plurality of wells, followed by loading of supports (e.g., beads) or droplets, including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a support (e.g., a bead such as a gel bead) or droplet. These supports or droplets can be loaded into a partition (e.g., a microwell) before, after, or concurrently with the loading of a cell, such that each cell is contacted with a different support (e.g., bead), or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or support can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecules may be attached to a droplet or bead that has been partitioned into the well. The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. In some instances, the nucleic acid barcode molecules may be releasable from the bead or droplet. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

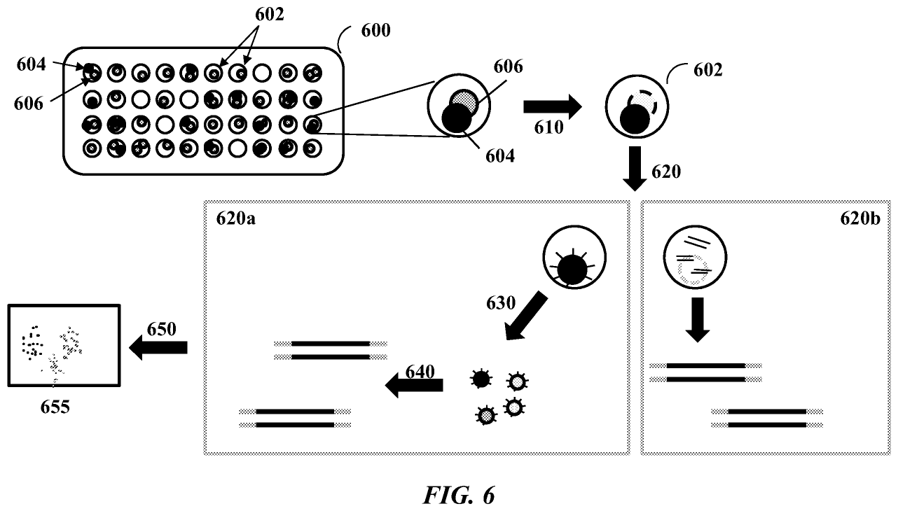

FIG. 6 schematically shows an example workflow for processing nucleic acid molecules within a sample. A substrate 600 including a plurality of microwells 602 can be provided. A sample 606 which can include a cell, cell bead, cellular components or analytes (e.g., proteins and/or nucleic acid molecules) can be co-partitioned, in a plurality of microwells 602, with a plurality of beads 604 including nucleic acid barcode molecules. During a partitioning process, the sample 606 can be processed within the partition. For instance, in the case of live cells, the cell can be subjected to conditions sufficient to lyse the cells and release the analytes contained therein. In process 620, the bead 604 can be further processed. By way of example, processes 620a and 620b schematically illustrate different workflows, depending on the properties of the bead 604.

In 620a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 630, the beads 604 from multiple wells 602 can be collected and pooled. Further processing can be performed in process 640. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

In 620b, the bead includes nucleic acid barcode molecules that are releasably attached thereto, as described below. The bead can degrade or otherwise release the nucleic acid barcode molecules into the well 602; the nucleic acid barcode molecules can then be used to barcode nucleic acid molecules within the well 602. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

### Reagents

In accordance with certain aspects, biological particles (e.g., the cells expressing the known antibody or a candidate antibody, bound or unbound by an antigen) may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below. The methods and systems of the present disclosure may comprise microfluidic devices and methods of use thereof, which may be used for co-partitioning biological particles or biological particles with reagents. Such systems and methods are described in U.S. Patent Publication No. US/20190367997, which is herein incorporated by reference in its entirety for all purposes.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the analyte carriers described above, other reagents can also be co-partitioned with the analyte carriers, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated analyte carriers (e.g., a cell or a nucleus in a polymer matrix), the analyte carriers may be exposed to an appropriate stimulus to release the analyte carriers or their contents from a co-partitioned support (e.g., bead). For example, in some cases, a chemical stimulus may be co-partitioned along with an encapsulated analyte carrier to allow for the degradation of the support and release of the cell or its contents into the larger partition. In some cases, this stimulus may be the same as the stimulus described elsewhere herein for release of nucleic acid molecules (e.g., partition-specific molecules) from their respective support (e.g., bead). In alternative examples, this may be a different and non-overlapping stimulus, in order to allow an encapsulated analyte carrier to be released into a partition at a different time from the release of nucleic acid molecules (e.g., partition-specific barcode molecules) into the same partition. For a description of methods, compositions, and systems for encapsulating cells (also referred to as a “cell bead”), see, e.g., U.S. Pat. No. 10,428,326 and U.S. Pub. 20190100632, which are each incorporated by reference in their entirety.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases may include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos may include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., partition-specific molecules) that include partition-specific barcode sequences that can be attached to or otherwise associated with the nucleic acid contents of individual biological particle, or to other components of the biological particle, and particularly to fragments of those nucleic acids. The nucleic acid molecules (e.g., partition-specific barcode molecules) are partitioned such that as between nucleic acid molecules (e.g., partition-specific barcode molecules) in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., partition-specific molecules). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, e.g., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules (e.g., partition-specific barcode molecules) can also include other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down partition-specific barcode molecules, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, supports, such as beads, are provided that each include large numbers of the above described partition-specific barcode molecules releasably attached to the beads, where all of the nucleic acid molecules (e.g., partition-specific barcode molecules) attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules (e.g., partition-specific barcode molecules) into the partitions, as they are capable of carrying large numbers of nucleic acid molecules (e.g., partition-specific barcode molecules), and may be configured to release those nucleic acid molecules (e.g., partition-specific barcode molecules) upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

Additionally, each bead can be provided with large numbers of nucleic acid (e.g., partition-specific barcode molecules) molecules attached. In particular, the number of molecules of nucleic acid molecules (e.g., partition-specific barcode molecules) including the barcode sequence on an individual bead can be at least about 1,000 nucleic acid molecules (e.g., partition-specific barcode molecules), at least about 5,000 nucleic acid molecules (e.g., partition-specific barcode molecules), at least about 10,000 nucleic acid molecules (e.g., partition-specific barcode molecules), at least about 50,000 nucleic acid molecules (e.g., partition-specific barcode molecules), at least about 100,000 nucleic acid molecules (e.g., partition-specific barcode molecules), at least about 500,000 nucleic acids, at least about 1,000,000 nucleic acid molecules (e.g., partition-specific barcode molecules), at least about nucleic acid molecules (e.g., partition-specific barcode molecules), at least about nucleic acid molecules (e.g., partition-specific barcode molecules), at least about nucleic acid molecules (e.g., partition-specific barcode molecules), at least about 100,000,000 nucleic acid molecules (e.g., partition-specific barcode molecules), at least about 250,000,000 nucleic acid molecules (e.g., partition-specific barcode molecules) and in some cases at least about 1 billion nucleic acid molecules (e.g., partition-specific barcode molecules), or more.

Nucleic acid molecules (e.g., partition-specific barcode molecules) of a given bead can include identical (or common) barcode sequences, different barcode sequences, or a combination of both. Nucleic acid molecules (e.g., partition-specific barcode molecules) of a given bead can include multiple sets of nucleic acid molecules (e.g., partition-specific barcode molecules). Nucleic acid molecules (e.g., partition-specific barcode molecules) of a given set can include identical barcode sequences. The identical barcode sequences can be different from barcode sequences of nucleic acid molecules (e.g., partition-specific barcode molecules) of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules (e.g., partition-specific barcode molecules), at least about 5,000 nucleic acid molecules (e.g., partition-specific barcode molecules), at least about 10,000 nucleic acid molecules (e.g., partition-specific barcode molecules), at least about 50,000 nucleic acid molecules (e.g., partition-specific barcode molecules), at least about 100,000 nucleic acid molecules (e.g., partition-specific barcode molecules), at least about 500,000 nucleic acids, at least about 1,000,000 nucleic acid molecules (e.g., partition-specific barcode molecules), at least about 5,000,000 nucleic acid molecules (e.g., partition-specific barcode molecules), at least about 10,000,000 nucleic acid molecules (e.g., partition-specific barcode molecules), at least about 50,000,000 nucleic acid molecules (e.g., partition-specific barcode molecules), at least about 100,000,000 nucleic acid molecules (e.g., partition-specific barcode molecules), at least about 250,000,000 nucleic acid molecules (e.g., partition-specific barcode molecules) and in some cases at least about 1 billion nucleic acid molecules (e.g., partition-specific barcode molecules).

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., partition-specific barcode molecules) are releasable from the beads upon the application of a particular stimulus to the beads. In some cases, the stimulus may be a photo-stimulus, e.g., through cleavage of a photo-labile linkage that releases the nucleic acid molecules (e.g., partition-specific barcode molecules). In other cases, a thermal stimulus may be used, where elevation of the temperature of the beads environment will result in cleavage of a linkage or other release of the nucleic acid molecules (e.g., partition-specific barcode molecules) from the beads. In still other cases, a chemical stimulus can be used that cleaves a linkage of the nucleic acid molecules (e.g., partition-specific barcode molecules) to the beads, or otherwise results in release of the nucleic acid molecules (e.g., partition-specific barcode molecules) from the beads. In one case, such compositions include the polyacrylamide matrices described above for encapsulation of biological particles, and may be degraded for release of the attached nucleic acid molecules (e.g., partition-specific barcode molecules) through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

In the methods and systems described herein, one or more labelling agents capable of binding to or otherwise coupling to one or more cell features may be used to characterize cells and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

Contacting with Reporter Oligonucleotide Conjugated Antigens

In some embodiments, the methods provided herein comprise obtaining a first epitope specificity of a first plurality of cells and a second epitope specificity of a second plurality of cells. In some embodiments, the epitope specificities are obtained by contacting an antigen with the first plurality of cells and the second plurality of cells. In some embodiments, the cells (e.g., the first plurality of cells and the second plurality of cells) are engineered to express at least one antigen binding molecule (e.g., the known antibody and the candidate antibody).

In some embodiments, contacting a plurality of cells expressing at least one antigen binding molecule with a reporter oligonucleotide conjugated antigen can include contacting the plurality of cells with the antigen such that the antigen can interact with the antigen binding molecule (e.g., the known antibody and the candidate antibody). In some embodiments, upon contacting, the antigen can bind to the antigen binding molecule (e.g., the known antibody and the candidate antibody).

Contacting can occur in a vessel, such as a well, a tube, a bead, a partition, a microfluidic system, or in another vessel. In some embodiments, contacting can occur on a membrane or a column. In some embodiments, contacting can occur on a surface or support, such as on a slide, a plate, or a dish.

Contacting can include exposing the antigen binding molecule (e.g., the known antibody and the candidate antibody) to the antigen under conditions that can allow binding if the antigen binding molecule has affinity for the antigen. In some embodiments, contacting can occur under physiological conditions, or under conditions that approximate physiological conditions.

In some embodiments, contacting can be performed under conditions that do not significantly alter the structure and/or function of the antigen or the antigen binding molecule. In some embodiments, contacting can be performed under conditions that do not significantly alter (e.g., increase or decrease) the affinity of the antigen binding molecule for the antigen. Conditions can include a pH, a temperature, or other conditions.

In some embodiments, contacting can occur in a buffer having a pH. In some embodiments, for example, contacting can occur in a buffer having a pH of about 4.5, about about 5.5, about 6.0, about 6.5, about 7.0, about 7.5, about 8.0, about 8.5, about 9.0, or a range between any two foregoing values.

In some embodiments, contacting can occur at a temperature that can allow protein-protein interactions. In some embodiments, contacting can occur at a temperature of about 10° C., about 15° C., about 20° C., about 25° C., about 30° C., about 35° C., about 40° C., about about 50° C., or a range between any two foregoing values.

In some embodiments, a method can include contacting the composition comprising at least one antigen binding molecule with a plurality of antigens, wherein the antigens can be conjugated to reporter oligonucleotides. In some such cases, an antigen of the plurality of antigens can be conjugated to a reporter oligonucleotide that is different than a reporter second oligonucleotide that is conjugated to a second antigen. In some embodiments, one or more such antigens can bind one or more antigen binding molecules in the composition. In some embodiments, one antigen can bind an antigen binding molecule in the composition more strongly than a second antigen.

In some embodiments, a method can include contacting the plurality of cells expressing at least one antigen binding molecule (e.g., the known antibody and the candidate antibody) with a plurality of variations of an antigen, wherein the variations of antigens can be conjugated to reporter oligonucleotides. In some such cases, a variation of the plurality of variations of an antigen can be conjugated to a reporter oligonucleotide that is different than a second reporter oligonucleotide that is conjugated to a second variation of the antigen.

Variations of an antigen can include, for example, an amino acid mutation (e.g., an insertion, deletion, or point mutation) or differences in glycosylation or other modification to an amino acid. In some embodiments, a variation of an antigen can include a non-natural amino acid. In some embodiments, a variation can, for example, be a variation of a therapeutic (e.g., an antibody or antibody-based drug) that is modified. In some embodiments, a modified therapeutic can be modified to reduce recognition of the therapeutic by the immune system of a subject. In some embodiments, a modified therapeutic can be modified to reduce binding of the therapeutic to an antigen binding molecule, such as an antibody or antigen binding fragment thereof.

### Partitioning Barcoded Antigen Binding Molecules

Antigen binding molecules (e.g., the known antibodies and/or the candidate antibodies) can be analyzed and/or isolated based at least in part on reporter oligonucleotides conjugated to antigens. In some embodiments, such analysis and/or isolation can facilitate identification and/or characterization of an antigen binding molecule that has affinity for the antigen.

In some instances, analyzing and/or isolating an antigen binding molecule bound to an antigen (such as a BCR of a B cell or an anti-drug antibody (ADA) bound to a therapeutic antibody or antibody-drug conjugate) can include hybridizing a reporter oligonucleotide of a reporter oligonucleotide conjugated antigen to a partition-specific barcode molecule to isolate or otherwise separate the antigen binding molecule from other materials (e.g., non-antigen bound materials).

Isolating an antigen binding molecule can include capturing the reporter oligonucleotide. In some embodiments, capturing the reporter oligonucleotide can include annealing the reporter oligonucleotide to a partition-specific barcode molecule. In some embodiments, a partition-specific barcode molecule can include one or more of a partition-specific barcode, a unique molecular identifier, or a template switching oligonucleotide as described elsewhere herein.

In some embodiments, isolating an antigen binding molecule can include pulling down the at least one antigen binding molecule using the partition-specific barcode molecule. In some embodiments, the partition-specific barcode molecule can be attached to a bead, such as those described elsewhere herein. In some embodiments, the partition-specific barcode molecule can be affixed to a slide, affixed to a well, affixed to a tube, or conjugated to a magnetic molecule.

In some embodiments, an antigen binding molecule can be isolated using a pull-down assay. For example, the oligonucleotide can be hybridized to a partition-specific barcode molecule that is conjugated to a protein or a magnetic particle. Upon hybridization, the protein or magnetic particle can be used to separate the antigen binding molecule from other components of the composition, for example by contacting the protein or magnetic particle with a binding partner to the protein or a magnetic field, respectively, and washing away other components of the composition. In some embodiments, for example when an antigen binding molecule is on a cell surface, the cell can be lysed prior to performing such a pull-down assay.

In some embodiments, for example when an antigen binding molecule is on a cell surface, the cell can be lysed or the binding molecule can otherwise be removed from the cell. Such lysis or removal can occur before or after the antigen binding molecule is isolated using a partition. In some embodiments, an antigen binding molecule can be isolated from a composition with the cell (e.g., the cell can remain intact). In some embodiments, an antigen binding molecule can be isolated using a flow cytometry technique.

In some embodiments, an antigen binding molecule can be isolated using a partition. In some cases, the antigen binding molecule bound to the reporter oligonucleotide conjugated antigen can be separated into a partition, for example a bead or another partition provided herein (e.g., droplet or well-based partitioning systems). A partition can include a partition-specific barcode molecule, which can be configured to hybridize to a reporter oligonucleotide conjugated to the antigen.

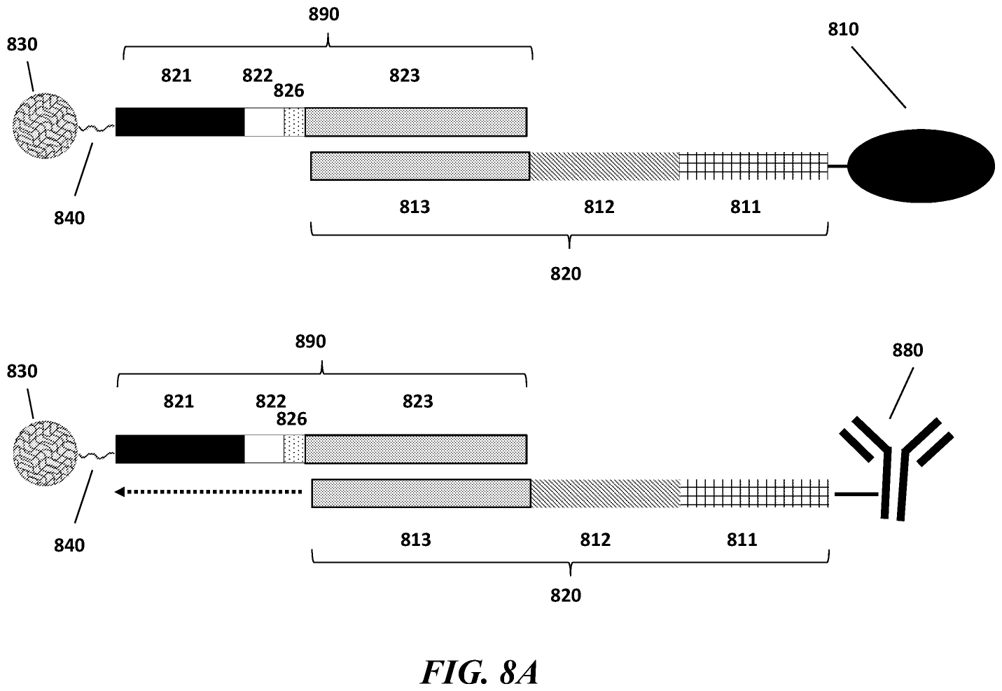

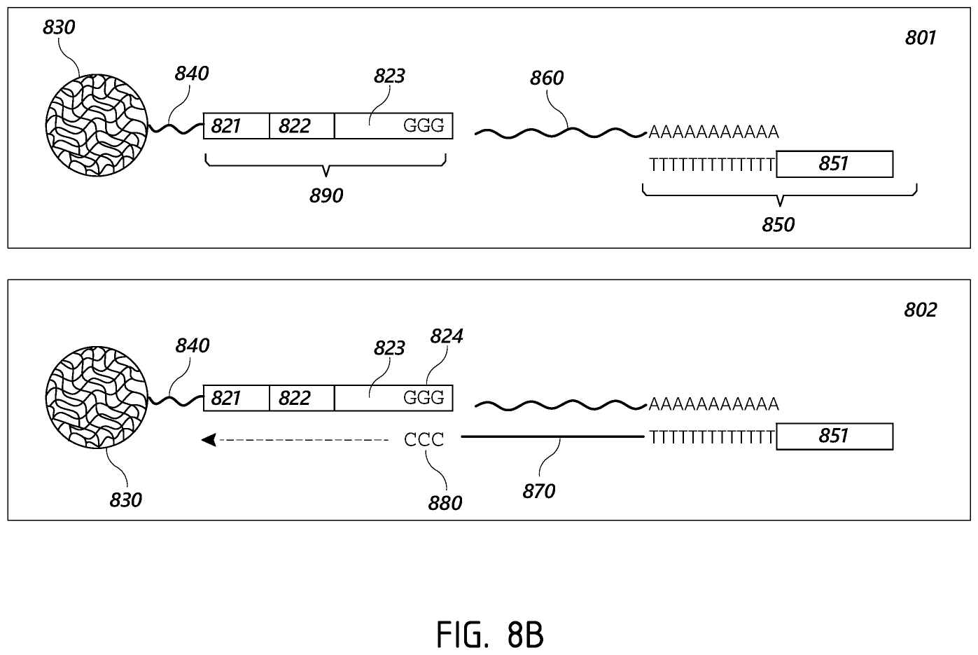

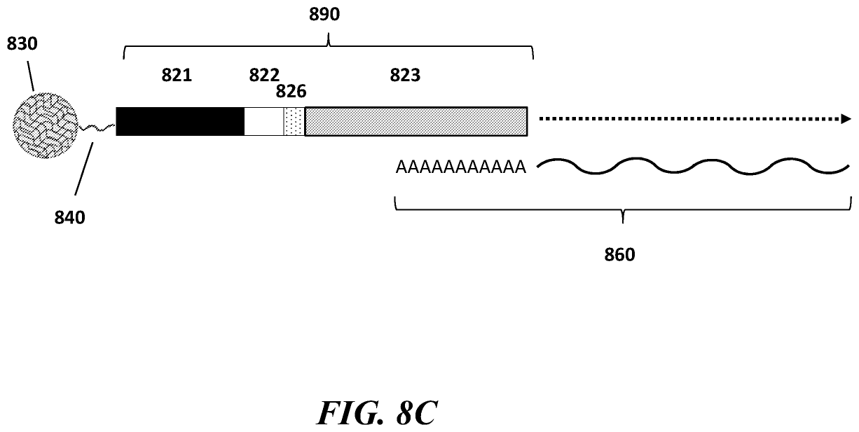

In some instances, analysis of one or more antigen binding molecules (e.g., using the oligonucleotide labeled agents (e.g., antigens) described herein) includes a workflow as generally depicted in FIGS. 8A-8C. For example, in some embodiments, cells are contacted with one or more reporter oligonucleotide 820 conjugated labelling agents (e.g., antigens) 810 (e.g., polypeptide, antibody (880), or pMHC molecule or complex) and optionally further processed prior to barcoding. Optional processing steps can include one or more washing and/or cell sorting steps. In some instances, a cell bound to labelling agent (e.g., antigen) 810 (e.g., polypeptide, antibody (880), or pMHC molecule or complex) conjugated to reporter oligonucleotide 820 and support 830 (e.g., a bead, such as a gel bead) conjugated to partition-specific barcode molecules 890 are partitioned into a partition amongst a plurality of partitions (e.g., a droplet of a droplet emulsion or a well of a micro/nanowell array). In some instances, the partition includes at most a single cell bound to labelling agent 810. In some embodiments, partition-specific barcode molecules 890 is attached to support 830 via a releasable linkage 840 (e.g., comprising a labile bond) as described elsewhere herein.

With continued reference to FIG. 8A, in some instances, oligonucleotide 820 conjugated to labelling agent (e.g., antigen) 810 (e.g., polypeptide, an antibody (880), pMHC molecule such as an MHC multimer, etc.) includes a functional sequence 811 (e.g., an adaptor sequence), a reporter barcode sequence 812 that identifies the labelling agent (e.g., antigen) 810 (e.g., the polypeptide, antibody (880), or peptide of a pMHC molecule or complex), and capture handle 813. Capture handle 813 can be configured to hybridize to a capture sequence, such as capture sequence 823 present on partition-specific barcode molecule 890. A capture handle can include a sequence that is complementary to a capture sequence on a partition-specific barcode molecule. In some instances, partition-specific barcode molecule 890 is attached to a support 830 (e.g., a bead, such as a gel bead), such as those described elsewhere herein. For example, partition-specific barcode molecule 890 can be attached to support 830 via a releasable linkage 840 (e.g., comprising a labile bond), such as those described elsewhere herein. In some instances, reporter oligonucleotide 820 includes one or more additional functional sequences, such as those described above. In some embodiments, the partition-specific barcode molecule 890 includes a UMI (826).

Referring to FIGS. 8B-8C, in some instances, nucleic acid molecules (e.g., partition-specific barcode molecules) derived from a cell (such as RNA molecules) can be similarly processed to append the partition-specific barcode sequence 822 to these molecules or derivatives thereof (e.g., cDNA molecules). For example, referring to FIG. 8B, in some embodiments, primer 850 includes a sequence complementary to a sequence of RNA molecule 860 (such as an RNA encoding for a BCR sequence) from a cell. In some instances, primer 850 includes one or more adapter sequences 851 that are not complementary to RNA molecule 860. In some instances, primer 850 includes a poly-T sequence. In some instances, primer 850 includes a sequence complementary to a target sequence in an RNA molecule. In some instances, primer 850 includes a sequence complementary to a region of an immune molecule, such as the constant region of a TCR or BCR sequence. Primer 850 is hybridized to RNA molecule 860 and cDNA molecule 870 is generated in a reverse transcription reaction. In some instances, the reverse transcriptase enzyme is selected such that several non-templated bases 880 (e.g., a poly-C sequence) are appended to the cDNA. Partition-specific barcode molecule 890 includes a sequence 824 complementary to the non-templated bases, and the reverse transcriptase performs a template switching reaction onto partition-specific barcode molecules 890 to generate a nucleic acid molecule including partition-specific barcode 822 (or a reverse complement thereof) and a sequence of cDNA 870 (or a portion thereof). In another example, referring to FIG. 8C, in some embodiments, partition-specific barcode molecule 890 includes capture sequence 823 complementary to a sequence of RNA molecule 860 from a cell. In some instances, capture sequence 823 includes a sequence specific for an RNA molecule. In some instances, capture sequence 823 includes a poly-T sequence. In some instances, capture sequence 823 includes a sequence specific for an RNA molecule. In some instances, capture sequence 823 includes a sequence complementary to a region of an immune molecule, such as the constant region of a TCR or BCR sequence. Capture sequence 823 can be hybridized to RNA molecule 860 and a cDNA molecule 870 (FIG. 8B) is generated in a reverse transcription reaction, generating a nucleic acid molecule including partition-specific barcode sequence 822 (or a reverse complement thereof) and a sequence of cDNA 870 (FIG. 8B) (or a portion thereof). This nucleic acid molecules (e.g., barcoded nucleic acid molecules) including partition-specific barcode 822 can then be optionally processed as described elsewhere herein, e.g., to amplify the molecules and/or append sequencing platform specific sequences to the fragments. See, e.g., U.S. Pat. Pub. 20180105808, which is hereby incorporated by reference in its entirety. The barcoded nucleic acid molecules, or derivatives generated therefrom, can then be sequenced on a suitable sequencing platform.

Exemplary partition-specific barcode molecules attached to a support (e.g., a bead) is shown in FIG. 10. In some embodiments, partition-specific barcode molecule 1010 can include functional sequence 1011, partition-specific barcode sequence 1012 and capture sequence 1013. Partition-specific barcode molecule 1020 can include functional sequence 1021, partition-specific barcode sequence 1012, and capture sequence 1023, wherein capture sequence 1023 includes a different sequence than capture sequence 1013. In some instances, functional sequence 1011 and functional sequence 1021 include the same sequence. In some instances, functional sequence 1011 and functional sequence 1021 include different sequences. Although support 1050 is shown including partition-specific barcode molecule 1010 and 1020, any suitable number of partition-specific barcode molecules including partition-specific barcode sequence 1012 are contemplated herein. For example, in some embodiments, support 1050 further includes partition-specific barcode molecule 1030. Partition-specific barcode molecule 1030 can include functional sequence 1031, partition-specific barcode sequence 1012 and capture sequence 1033, wherein capture sequence 1033 includes a different sequence than capture sequence 1013 and 1023. In some instances, partition-specific barcode molecules (e.g., 1010, 1020, 1030) include one or more additional functional sequences, such as a UMI or other sequences described herein.

### Library Preparation

In certain embodiments, each of the plurality of single cell suspensions comprises one of either the first plurality of cells or the second plurality of cells bound to or not bound to the antigen. In some embodiments, the first and the second plurality of cells are presented as cell beads. In some embodiments, the antigen is labeled with a reporter oligonucleotide as described herein. In some embodiments, the reporter oligonucleotide comprises a reporter oligonucleotide. In specific embodiments, the reporter oligonucleotide comprises at least one reporter capture handle, at least one reporter barcode (e.g., a barcode sequence), and/or at least one functional sequence as described in detail below. In some embodiments, following the contacting the antigen with the first plurality of cells and the second plurality of cells, the first plurality of cells and the second plurality of cells can be partitioned into a plurality of partitions, wherein a partition of the plurality of partitions includes a cell of the first plurality of cells or the second plurality of cells bound to the antigen, and a plurality of nucleic acid barcode molecules wherein a first nucleic acid barcode molecule of the plurality of nucleic acid barcode molecules includes a partition barcode sequence.

In some embodiments, the methods comprise generating a library of barcoded nucleic acid molecules from the first plurality of cells and the second plurality of cells, wherein the library of barcoded nucleic acid molecules comprises the reporter sequence or complement thereof and sequences corresponding to immune receptors. In some embodiments, in the partition, the first nucleic acid barcode molecule is coupled to the reporter oligonucleotide, and the reporter oligonucleotide coupled to the first nucleic acid barcode molecule is used to generate a first barcoded nucleic acid molecule comprising the reporter sequence or a reverse complement thereof and the partition barcode sequence or a reverse complement thereof. In some embodiments, a second nucleic acid barcode molecule of the plurality of nucleic acid barcode molecules comprises the partition barcode sequence, and generating a library of barcoded nucleic acid molecules from the first plurality of cells and the second plurality of cells further includes, in the partition, coupling the second nucleic acid barcode molecule to a nucleic acid analyte of the cell bound to the antigen, the nucleic acid analyte comprising a sequence of an immune receptor expressed by the cell bound to the antigen, or a reverse complement thereof, and using the nucleic acid analyte of the cell bound to the antigen coupled to the second nucleic acid barcode molecule to generate a second barcoded nucleic acid molecule comprising the sequence of the immune receptor expressed by the cell bound to the antigen, or a reverse complement thereof. In some embodiments, the methods further comprise sequencing the library of barcoded nucleic acid molecules. Methods for preparing the reporter library, the V(D)J library, and the B cell receptor library can be done by methods generally known in the art, such as the Cell Surface Protein Labeling for Single Cell RNA Sequencing Protocols with Feature Barcoding technology (10X Genomics, CG000149, Rev B), the content of which is incorporated herein by reference in its entirety.

In some embodiments, the plurality of nucleic acid barcode molecules is attached to a bead, and the partition barcode sequence identifies the bead. In some embodiments, the first nucleic acid barcode molecule comprises a first capture sequence configured to couple to the reporter oligonucleotide. The reporter oligonucleotide can further include a capture handle sequence complementary to the first capture sequence.

In some embodiments, the second nucleic acid barcode molecule further includes a second capture sequence configured to couple to the nucleic acid analyte of the cell bound to the antigen. The nucleic acid analyte can be an mRNA analyte or a cDNA molecule generated from the mRNA analyte.

In some embodiments, the second capture sequence is a template switch oligonucleotide sequence. In some embodiments, the first capture sequence and the second capture sequence are identical. Alternatively, the first capture sequence and the second capture sequence can be different.

In some embodiments the partition is a droplet. The partition can also be a well.

In some embodiments, the methods described herein further involve contacting the first plurality of cells with a first cell group labeling agent comprising a first cell group reporter oligonucleotide, the first cell group reporter oligonucleotide comprising a first cell group reporter sequence that identifies the first plurality of cells, and contacting the second plurality of cells with a second cell group labeling agent, the second cell group labeling agent comprising a second cell group reporter oligonucleotide comprising a second cell group reporter sequence that identifies the second plurality of cells.

In some embodiments, a third nucleic acid barcode molecule of the plurality of nucleic acid barcode molecules includes the partition barcode sequence and generating a library of barcoded nucleic acid molecules from the first plurality of cells and the second plurality of cells further includes, in the partition, coupling the third nucleic acid barcode molecule to the first cell group reporter oligonucleotide or to the second cell group reporter oligonucleotide, and using the third nucleic acid barcode molecule coupled to the first cell group reporter oligonucleotide or to the second cell group reporter oligonucleotide to generate a third barcoded nucleic acid molecule comprising the first cell group reporter sequence or the second cell group reporter sequence, or a reverse complement thereof, and the partition barcode sequence or a reverse complement thereof.

In some embodiments, the methods further comprise determining a sequence of the first barcoded nucleic acid molecule or a derivative thereof, the second barcoded nucleic acid molecule or a derivative thereof, and/or the third barcoded nucleic acid molecule or a derivative thereof.

### Identifying Antigen Binding Molecules

In some embodiments, the methods provided herein comprise identifying the epitope specificity of the antigen binding molecules (e.g., the known antibodies and the candidate antibodies). Identifying an antigen binding molecule can include determining the identity of the antigen binding molecule. In some embodiments, an antigen binding molecule can be determined by comparing the antigen binding molecule to a library of antigen binding molecules, by sequencing the antigen binding molecule, using imaging, using histochemical techniques, using spectrometry, or by another technique. In some embodiments, an antigen binding molecule can be identified using a barcode associated with the reporter oligonucleotide (e.g., a reporter barcode, such as a barcode sequence, on the oligonucleotide conjugated to the antigen, or a partition-specific barcode appended to a partition-specific barcode molecule complementary to the reporter oligonucleotide conjugated to the antigen).

In some embodiments, the sequence (e.g., the amino acid sequence) of the antigen binding molecule can be determined. In some embodiments, the sequence of the entire antibody can be determined. In some embodiments, a portion of the sequence of the antibody, such as the variable region of the light chain or a fragment thereof, the variable region of the heavy chain or a fragment thereof, the constant region of the light chain or a fragment thereof, the constant region of the heavy chain or a fragment thereof, or a combination thereof can be determined. This can be accomplished, for example, by using mass spectrometry or by Edman degradation using a protein sequenator (sequencer).

In some embodiments, an isotype of the antigen binding molecule (e.g., IgA, IgD, IgG, IgE, or IgM) can be determined. Isotype can be determined, for example, by sequencing or by an assay designed to determine the isotype (e.g., an antibody based assay or other assay).

In some embodiments, the antigen binding molecule can be identified based on its affinity to an epitope of the antigen. In some such cases, the sequence of the antigen can be known, and techniques such as epitope mapping techniques can be implemented to identify the antigen binding molecule.

In some embodiments, more than one antigen binding molecules can be identified. For example, a sample from a subject can include more than one antigen binding molecule that can bind to an antigen (e.g., a therapeutic antibody) that can be identified by methods described herein. In some such cases, a first identified antigen binding molecule can have a higher affinity for the antigen than a second identified antigen binding molecule. The more than one antigen binding molecule can bind to the same epitope on the antigen or to different epitopes on the antigen. In some embodiments, 2, 3, 4, 5, 10, 50, 100, or more antibodies can be identified.

### Determining an Epitope

In some embodiments, methods provided herein can include identifying a site on the antigen binding to the antigen binding molecule. Identification of a site can include identification of at least one amino acid(s) that contribute of binding of the antigen to the antigen binding molecule.

In some embodiments, methods provided herein can include identifying an epitope on an antigen that has affinity to the antigen binding molecule. Such an epitope can partially or fully confer affinity of the antigen binding molecule to the antigen.

An epitope can be a portion of an antigen or other macromolecule capable of forming a binding interaction with the variable region binding pocket of an antigen binding molecule such as an antibody or antigen binding fragment thereof. Such binding interactions can be manifested as an intermolecular contact with one or more amino acid residues of one or more CDRs. Antigen binding can involve, for example, a CDR3, a CDR3 pair or, in some cases, interactions of up to all six CDRs of the V_{H} and V_{L} chains. An epitope can be a linear peptide sequence (e.g., “continuous”) or can be composed of noncontiguous amino acid sequences (e.g., “conformational” or “discontinuous”). An antigen binding molecule, such as a known antibody or a candidate antibody, can recognize one or more amino acid sequences; therefore, an epitope can define more than one distinct amino acid sequence. Epitopes recognized by antigen binding molecules such as antibodies or antigen binding fragments thereof can be determined by peptide mapping or sequence analysis techniques. In some embodiments, binding interactions can be manifested as intermolecular contacts between an epitope on an antigen and one or more amino acid residues of a CDR. Epitopes recognized by antigen binding molecules such as antibodies or antigen binding fragments thereof can be determined, for example, by peptide mapping or sequence analysis techniques. Binding interactions can manifest as intermolecular contacts between an epitope on an antigen and one or more amino acid residues of a complementarity determining region (CDR).

An epitope can be determined, for example, using one or more epitope mapping techniques. Epitope mapping can include experimentally identifying the epitope on an antigen. Epitope mapping can be performed by any acceptable method, for example X-ray co-crystallography, cryogenic electron microscopy, array-based oligo-peptide scanning, site-directed mutagenesis mapping, high-throughput shotgun mutagenesis epitope mapping, hydrogen-deuterium exchange, cross-linking coupled mass spectrometry, yeast display, phage display, proteolysis, or a combination thereof.

### Systems for Epitope Binning

“Epitope binning” as used herein generally refers to a competitive immunoassay used to characterize and sort a library of antibodies against a target antigen. Generally, in epitope binning, antibodies against a similar target antigen are tested in pairwise against all other antibodies in a library of antibodies to determine which antibodies block the same epitopes on a target antigen. After each antibody has a profile created against all of the other antibodies in the library, a competitive blocking profile is created for each antibody relative to the others in the library. Closely related binning profiles indicate that the antibodies bind to the same or a closely related epitope and are “binned” together.

In some embodiments, the methods provided herein include obtaining a first epitope specificity of a first plurality of cells engineered to express a known antibody. The methods further include obtaining a second epitope specificity of a second plurality of cells engineered to express a candidate antibody. In some embodiments, the first epitope specificity and the second epitope are obtained by contacting an antigen with the first plurality of cells and the second plurality of cells. In some embodiments, the antigen is labeled with a reporter oligonucleotide.

In some embodiments, the methods provided herein comprise generating, a first reduced dimension representation of the first epitope specificity of the first plurality of cells expressing the known antibody, and a second reduced dimension representation of the second epitope specificity of the second plurality of cells expressing the candidate antibody, as described herein in the following sections.

In some embodiments, the methods provided herein comprise determining, based at least on the first reduced dimension representation and the second reduced dimension representation, whether the second plurality of cells exhibit a novel epitope specificity or an epitope specificity associated with the first plurality of cells.

The present disclosure also provides computer systems configured to implement the various methods disclosed herein including, for example, methods to organize antibodies into “bins” corresponding to epitope specificity, identify antibodies targeting similar epitopes, identify a region of interest and set(s) of epitopes targeted by an antibody-based therapy, screen candidate antibodies, and/or the like. For example, the methods provided herein includes applying, to the first epitope specificity of the first plurality of cells engineered to express the known antibody and the second epitope specificity of the second plurality of cells engineered to express the candidate antibody, one or more data analysis techniques to determine whether the second plurality of cells exhibit a novel epitope specificity or an epitope specificity associated with the first plurality of cells.

In some example embodiments, the one or more data analysis techniques may be performed by at least one data processor included, for example, in a computer system. To further illustrate, FIG. 11 depicts a system diagram illustrating an example of an analysis system 1100, in accordance with some example embodiments. Referring to FIG. 11, the analysis system 1100 may include an analysis engine 1102, a sequencing platform 1104, and a client device 1106. As shown in FIG. 11, the analysis engine 1102, the sequencing platform 1104, and the client device 1106 may be communicatively coupled by a network 1105. The network 1105 may be a wired network and/or a wireless network including, for example, a local area network (LAN), a virtual local area network (VLAN), a wide area network (WAN), a public land mobile network (PLMN), the Internet, and/or the like.

Referring again to FIG. 11, in some example embodiments, the analysis engine 1102 may receive, from the sequencing platform 1104, data associated with a barcoded nucleic acid molecule containing sequence information from a partition-specific barcode molecule and a reporter oligonucleotide conjugated to each of a plurality of antigens. As noted, the reporter oligonucleotide conjugated to an antigen may include a sequence, such as a reporter barcode (e.g., a barcode sequence), that enables an identification of the antigen. Conjugating each of the plurality of antigens with a reporter oligonucleotide that includes a reporter barcode (e.g., a barcode sequence) may further enable a differentiation between different antigens, for example, during a multiplexed antigen assay. To further facilitate the processing and identification of the reporter barcode, such as a barcode sequence (e.g., through nucleic acid sequencing), the reporter oligonucleotide may be coupled with a partition-specific barcode molecule that includes one or more of a partition-specific barcode, and a template switching oligonucleotide (TSO) site.

In some example embodiments, the analysis engine 1102 may receive, from the sequencing platform 1104, data corresponding to a first epitope specificity of a first plurality of cells expressing a known antibody and a second epitope specificity of a second plurality of cells expressing a candidate antibody. As used herein, the “epitope specificity” of an antibody may correspond to the epitopes towards which the antibody exhibits a binding affinity. Moreover, antibodies, such as monoclonal antibodies, that target similar epitopes may share a similar function. As such, organizing known antibodies and candidate antibodies into epitope bins based on the respective epitope specificity of the antibodies may enable a determination of the epitope specificity of the candidate antibodies. For example, a candidate antibody that is organized into a same epitope bin as a known antibody may be determined to exhibit a same epitope specificity as the known antibody. The candidate antibody may therefore be determined to target similar epitopes and share similar functions as the known antibody. Contrastingly, a candidate antibody that is organized into a separate epitope bin as the known antibodies may be determined to exhibit a novel epitope specificity that is unlike the epitope specificity of any of the known antibodies. Such a candidate antibody may not target similar epitopes or share similar functions as any of the known antibodies.

To further illustrate, FIG. 12A depicts an example of a dataset 1000 representative of an epitope specificity of known antibodies and candidate antibodies, in accordance with some example embodiments. The dataset 1000 may include the epitope specificity of an n quantity of known antibodies (e.g., Ab #1, Ab #2, Ab #3, Ab #4, . . . , Ab #n) as well as the epitope specificity of a candidate antibody (e.g., candidate Ab). For example, as shown in FIG. 12A, the dataset 1000 may include an indication of whether each of the n quantity of known antibodies and the candidate antibody binds to one or more antigens.

In some example embodiments, the analysis engine 1102 may derive, based at least on the dataset 1000, one or more corresponding count matrices indicating a quantity of times an antigen bound to an antibody (or a cell expressing the antibody). To further illustrate, FIG. 12B depicts an example of a count matrix 1210 having an n quantity of rows corresponding to an n quantity of antigens and an m quantity of columns m quantity of antibodies (or cells expressing an antibody). Each element in the count matrix 1210 may correspond to a count of a quantity of times each of the n quantity of antigens bound to one of the m quantity of antibodies (or cells expressing an antibody). For example, as shown in FIG. 12B, a first antigen Antigen 1 bound to an antibody expressed by a second cell Cell 2 ten times and an antigen expressed by a third cell Cell 3 thirty times. It should be appreciated that the count matrix 1210 may also contain, alternatively and/or additionally, information relating to surface protein (non-antigen), intracellular protein (non-antigen), gene expression (targeted or untargeted RNA), DNA accessibility/ATAC, metabolite data, CRISPR guide, DNA repair activity, and/or the like.

In some example embodiments, the analysis engine 1102 may derive, based at least on the dataset 1000, multiple count matrices. For example, the analysis engine 1102 may derive, based at least on a first portion of the dataset 1000, a first count matrix including the epitope specificity of a plurality of known antibodies (or a first plurality of cells expressing the known antibodies). The analysis engine 1102 may further derive, based at least on a second portion of the dataset 1000, a second count matrix including an epitope specificity of a plurality of candidate antibodies (or a second plurality of cells expressing the candidate antibodies).

The dataset 1000, including the one or more count matrices derived therefrom, may correspond to high dimensional data. For example, the dataset 1000 may have a quantity of dimensions corresponding to the quantity of antigens each cell expressing a known antibody or a candidate antibody is contacted with. The high dimensionality of the dataset 1000 may obscure the relationships between the different antibodies (or cells expressing the antibodies) included in the dataset 1000. For example, the high dimensionality of the dataset 1000 may obscure the similarities between cells expressing antibodies that exhibit a same epitope specificity by binding to a same or similar group of antigens. Moreover, the high dimensionality of the dataset 1000 may thwart efforts to analyze and visualize the dataset 1000. For instance, the high dimensionality of the dataset 1000 may cause overfitting, in which the analysis is overly biased by the noise present in the dataset 1000. The high dimensionality of the dataset 1000 may therefore prevent the identification of cells expressing antibodies having a same epitope specificity and/or a novel epitope specificity.

As such, in some example embodiments, the analysis engine 1102 may apply one or more machine learning based techniques to analyze the dataset 1000 and determine the epitope specificity of one or more candidate antibodies. For instance, the analysis engine 1102 may apply a semi-unsupervised machine learning technique that includes generating a reduced dimension representations of the dataset 1000 received from the sequencing platform 1104, such as the first count matrix including an epitope specificity of a plurality of known antibodies (or a first plurality of cells expressing the known antibodies) and the second count matrix including an epitope specificity of a plurality of candidate antibodies (or a second plurality of cells expressing the candidate antibodies) derived from the dataset 1000. The analysis engine 1102 may reduce the dimensionality of each count matrix by applying one or more supervised decomposition techniques and/or unsupervised decomposition techniques to decompose the count matrix 1210. Examples of decomposition techniques may include principal component analysis (PCA), neighborhood component analysis, linear discriminant analysis, and non-negative matrix factorization.

In some example embodiments, the analysis engine 1102 may generate a visualization 1145 of the dataset 1000 to enable further analysis of the dataset 1000. For example, the visualization 1145 may be a graphic user interface (GUI) displayed at the client 1106 showing clusters (or other groupings) of cells expressing antibodies having a same epitope specificity. Each of these clusters (or groupings) of cells may correspond to a single epitope bin containing cells expressing antibodies targeting similar epitopes. However, even the reduced dimension representation of the dataset 1000 may still be difficult to visualize and analyze because the reduced dimension representation of the dataset 1000 may still occupy a high-dimensional space (e.g., greater than two or three dimensions). As such, in order to generate the visualization 1145 of the dataset 1000, the analysis engine 1102 may embed, in a low dimensional space (e.g., a two-dimensional space or a three-dimensional space), the reduced dimension representation of the dataset 1000. For instance, the analysis engine 1102 may embed the reduced dimension representation of the dataset 1000 by applying a uniform manifold approximation and projection (UMAP), a T-distributed Stochastic Neighbor Embedding (t-SNE), a generalized principal component analysis (GLM-PCA), and/or the like.

To further illustrate, FIG. 12C depicts an example of the visualization 1145 including one or more clusters (or other groupings) of cells expressing antibodies having a same epitope specificity. For example, the example of the visualization 1145 shown in FIG. 12C includes a first cluster 1220a corresponding to cells expressing a first known antibody Ab #1, a second cluster 1220b corresponding to cells expressing a second known antibody Ab #2, a third cluster 1220c corresponding to cells expressing a third known antibody Ab #3, and a fourth cluster 1220d corresponding to cells expressing a fourth known antibody Ab #n.

The epitope specificity of the cells expressing a candidate antibody (e.g., candidate Ab), forming a cluster 1220e, may be determined based at least on a distribution of these cells relative to the first cluster 1220a, the second cluster 1220b, the third cluster 1220c, and the fourth cluster 1220d. For instance, in the example of the visualization 1145 shown in FIG. 12C, the cells expressing the candidate antibody that form part of the first cluster 1220a are determined to exhibit the same epitope specificity as the cells expressing the first known antibody Ab #1. That is, the analysis engine 1102 may determine, based at least on the distribution of the cells expressing the candidate antibody relative to the first cluster 1220a including the cells expressing the known antibody Ab #1, that these candidate antibodies target a same or similar group of epitopes as the known antibody Ab #1. Contrastingly, the cells expressing the candidate antibody that do not form a part of any one of the first cluster 1220a, the second cluster 1220b, the third cluster 1220c, and the fourth cluster 1220d may be determined to exhibit a novel epitope specificity. These candidate antibodies do not target a same or similar group of epitopes as the first known antibody Ab #1, the second known antibody Ab #2, the third known antibody Ab #3, or the fourth known antibody Ab #4.

In addition to the techniques described above, graph-based methods may also be applied to reduce the dimensionality of the dataset 1000 and embed the dataset 1000 in a low-dimensional space for visualization. As noted, reducing the dimensionality of the dataset 1000 and embedding the reduced dimension representation of the dataset 1000 may organize the cells expressing the known antibodies as well as the cells expressing the candidate antibodies into one or more epitope bins, each of which including cells exhibiting a same epitope specificity. Doing so may enable the identification of antibody-expressing cells that target similar epitopes and thus exhibit a same epitope specificity. Moreover, whether a candidate antibody exhibits a novel epitope specificity or a same epitope specificity as a known antibody may be determined based at least on the epitope bin associated with the candidate antibody.

In some example embodiments, the results of the dimensionality reduction and the embedding may be validated. For example, the analysis engine 1102 may validate the results of reducing the dimensionality of the dataset 1000 and embedding the reduced dimension representation of the dataset 1000 by applying one or more clustering algorithms such as a Leiden algorithm, a Louvain algorithm, a phenotyping by accelerated refined community partitioning (PARC) algorithm, a density-based spatial clustering of applications with noise (DBSCAN) algorithm, and/or the like.

In some example embodiments, instead of the semi-unsupervised machine learning technique described above, the analysis engine 1202 may apply a semi-supervised machine learning technique to analyze the dataset 1000 and determine the epitope specificity of the one or more candidate antibodies. Transfer learning is one example of a semi-supervised machine learning technique in which a machine learning model trained to perform a different but related task is applied to perform the task of determining the epitope specificity of the one or more candidate antibodies. For example, a machine learning model that is trained, using training data that includes labeled antibodies, to identify antibodies may be used in the transfer learning context to determine the epitope specificity of the one or more candidate antibodies.

Alternatively and/or additionally, the analysis engine 1102 may apply a supervised machine learning technique to analyze the dataset 1000 and determine the epitope specificity of one or more candidate antibodies. The analysis engine 1102 may apply, to the dataset 1000, one or more machine learning models including for example, a neural network, a Bayesian classifier, a decision tree, a logistic regression model, a k-nearest neighbor model, a support vector machine, and/or the like. The dataset 1000 in this case may include, for each known antibody and candidate antibody, a corresponding count vector in which each element correspond to a count of a quantity of times the antibody bound to an antigen. Prior to being input into the machine learning model, the analysis engine 1102 may encode the count vectors included in the dataset 1000 based, for example, on the properties of the protein sequence of each antibody and the corresponding antigen count. For example, the machine learning model may be a neural network that includes one or more layers configured to perform the encoding (e.g., one-hot encoding and/or the like) such that further analysis of the dataset 1000 is performed on the encoded count vectors. It should be appreciated that encoding the count vectors may improve the performance of the machine learning model in predicting, based at least on the count vector associated with an antibody, the epitope specificity of that antibody.

In some example embodiments, the count vectors associated with the known antibodies may serve as training data for training the machine learning model. Once trained, the machine learning model may be deployed to predict, based at least on the count vector of a candidate antibody, the epitope specificity of that candidate antibody. For example, the machine learning model may receive, as an input, the count vector of the candidate antibody. The machine learning model may generate a corresponding output that includes a probability of the candidate antibody belonging to each of one or more epitope bins.

As noted, each epitope bin may correspond to a particular epitope specificity. Accordingly, the candidate antibody may be determined to exhibit a same epitope specificity as a known antibody if the probability of the candidate antibody belonging to the epitope bin associated with the known antibody exceeds a threshold value. Contrastingly, if the probability of the candidate antibody belonging to each of the epitope bins fails to exceed a threshold value, the analysis engine 1102 may determine that the candidate antibody exhibits a novel epitope specificity.

In some example embodiments, the dataset 1000 may also be visualized based on a density distribution of an antigen binding score that is computed for each possible antigen that a cell expressing an antibody is exposed to. For example, the score for each antigen may be computed by transforming, for each known antibody and candidate antibody, a count vector in which each element corresponds to a count of a quantity of times the antibody bound to an antigen. The count vectors may be transformed by applying a log transform such as, for example, a centered log ratio transform and/or the like. Moreover, the count vectors may be transformed without or without centering and/or scaling the transformed count vectors.

To further illustrate, FIG. 12D depicts another example of the visualization 1145, in accordance with some example embodiments. The example of the visualization 1145 shown in FIG. 12D shows the distribution of antigen scores for known antibodies Ab #2, Ab #3, and Ab #4. If the antibody expressed by a cell binds to a given antigen, the score for the antigen may shift to the right of the distribution whereas the score may shift to the left if the antibody expressed by the cell does not bind to the given antigen. Accordingly, the distribution of these scores may be similar for cells expressing antibodies having a same epitope specificity. Cells expressing antibodies having the same epitope specificity may therefore be identified based on a comparison of the distribution of these antigen scores. For instance, the distribution of antigen scores may be compared by applying one or more tests for evaluating differences in distribution density (e.g., a Kolmogorov-Smirnov test and/or the like. Alternatively and/or additionally, outlier detection techniques may be applied to these distributions in order to identify “outliers” that correspond to cells expressing antibodies that have a novel epitope specificity.

FIG. 14 depicts a flowchart illustrating an example of a process 1450 for analyzing and visualizing data associated with cells expressing one or more antigen binding molecules, in accordance with some example embodiments. Referring to FIGS. 11, 12A-12D, 13 and 14, the process 1450 may be performed by the analysis engine 1102 to analyze, for example, the dataset 1000 and determine the epitope specificity of one or more candidate antibodies (or cells expressing the candidate antibodies).

At 1452, the analysis engine 1102 may generate, based at least on a first dataset including a first epitope specificity of a first plurality of cells expressing a known antibody, a first count matrix. In some example embodiments, the analysis engine 1102 may receive, from the sequencing platform 1104, the dataset 1000, which may be associated with a barcoded nucleic acid molecule containing sequence information from a partition-specific barcode molecule and a reporter oligonucleotide conjugated to each of a plurality of antigens. The reporter oligonucleotide conjugated to an antigen may include a unique sequence, such as a reporter barcode (e.g., a barcode sequence), that enables an identification of the antigen as well as a differentiation between different antigens. As shown in FIG. 12A, the dataset 1000 may include the epitope specificity of one or more known antibodies. Accordingly, the analysis engine 1102 may generate, based at least the dataset 1000, a first count matrix indicating a count of a quantity of times each of a plurality of antigens bound to each of a plurality of cells expressing one or more known antibodies. For instance, in the example of the count matrix 1210 shown in FIG. 12B, the first antigen Antigen 1 bound to the antibody expressed by the second cell Cell 2 ten times and the antigen expressed by the third cell Cell 3 thirty times.

At 1454, the analysis engine 1102 may generate, based at least on a second dataset including a second epitope specificity of a second plurality of cells expressing a candidate antibody, a second count matrix. In some example embodiments, the dataset 1000 may also include the epitope specificity of one or more candidate antibodies. As such, the analysis engine 1102 may also generate, based at least the dataset 1000, a second count matrix indicating a count of a quantity of times each of a plurality of antigens bound to each of a plurality of cells expressing one or more candidate antibodies.

At 1456, the analysis engine 1102 may generate a first reduced dimension representation of the first matrix and a second reduced dimension representation of the second matrix. As noted, the high dimensionality of the dataset 1000 may prevent the identification of cells expressing antibodies having a same epitope specificity and/or a novel epitope specificity. Thus, in some example embodiments, the analysis engine 1102 may apply one or more machine learning based techniques to analyze the dataset 1000 and determine the epitope specificity of one or more candidate antibodies. The analysis engine 1102 may apply a semi-unsupervised machine learning technique that includes generating a reduced dimension representations of the dataset 1000. For example, the analysis engine 1102 may reduce the dimensionality of each count matrix by applying one or more supervised decomposition techniques and unsupervised decomposition techniques to decompose the count matrix 1210. As noted, examples of decomposition techniques may include principal component analysis (PCA), neighborhood component analysis, linear discriminant analysis, and non-negative matrix factorization.

At 1458, the analysis engine 1102 may embed, in a low dimensional space, the first reduced dimension representation of the first count matrix and the second reduced dimension representation of the second count matrix. As noted, even the reduced dimension representation of the dataset 1000 may still be difficult to analyze and visualize because the reduced dimension representation of the dataset 1000 may still occupy a high-dimensional space (e.g., greater than two or three dimensions). As such, in order to generate the visualization 1145 of the dataset 1000, the analysis engine 1102 may embed, in a low dimensional space (e.g., a two-dimensional space or a three-dimensional space), the reduced dimension representation of the dataset 1000. For example, the analysis engine 1102 may embed the reduced dimension representation of the dataset 1000 by applying a uniform manifold approximation and projection (UMAP), a T-distributed Stochastic Neighbor Embedding (t-SNE), a generalized principal component analysis (GLM-PCA), and/or the like.

At 1460, the analysis engine 1102 may determine, based at least on the embedded first reduced dimension representation of the first count matrix and the embedded second reduced dimension representation of the second count matrix, whether the second plurality of cells exhibit a novel epitope specificity or an epitope specificity associated with the first plurality of cells. In one exemplification, the example of the visualization 1145 shown in FIG. 12C includes the first cluster 1220a corresponding to cells expressing the first known antibody Ab #1, the second cluster 1220b corresponding to cells expressing the second known antibody Ab #2, the third cluster 1220c corresponding to cells expressing the third known antibody Ab #3, and the fourth cluster 1220d corresponding to cells expressing the fourth known antibody Ab #n. The epitope specificity of the cells expressing a candidate antibody (e.g., candidate Ab) may be determined based at least on a distribution of these cells relative to the first cluster 1220a, the second cluster 1220b, the third cluster 1220c, and the fourth cluster 1220d. For instance, in the example of the visualization 1145 shown in FIG. 12C, the cells expressing the candidate antibody that form part of the first cluster 1220a are determined to exhibit the same epitope specificity as the cells expressing the first known antibody Ab #1. Contrastingly, the cells expressing the candidate antibody that do not form a part of any one of the first cluster 1220a, the second cluster 1220b, the third cluster 1220c, and the fourth cluster 1220d may be determined to exhibit a novel epitope specificity. Accordingly, these candidate antibodies do not target similar epitopes as the first known antibody Ab #1, the second known antibody Ab #2, the third known antibody Ab #3, or the fourth known antibody Ab #4.

In some embodiments, a method for determining epitope specificity is provided. In some embodiments, the method comprises obtaining a first epitope specificity of a first plurality of cells and a second epitope specificity of a second plurality of cells by contacting an antigen with the first plurality of cells and the second plurality of cells, wherein the antigen is labeled with a reporter oligonucleotide comprising a reporter sequence, and wherein the first plurality of cells is engineered to express a known antibody and the second plurality of cells is engineered to express a candidate antibody. The method also comprises generating, a first reduced dimension representation of the first epitope specificity of the first plurality of cells expressing the known antibody; and a second reduced dimension representation of the second epitope specificity of the second plurality of cells expressing the candidate antibody. The method further comprises determining, based at least on the first reduced dimension representation and the second reduced dimension representation, whether the second plurality of cells exhibit a novel epitope specificity or an epitope specificity associated with the first plurality of cells.

In some embodiments, a system for determining epitope specificity is provided. In some embodiments, the system can include at least on data processor, and at least one memory storing instructions, which when executed by the at least one data processor, result in operations comprising generating, based at least on a first dataset indicating a first epitope specificity of a first plurality of cells expressing a known antibody, a first reduced dimension representation of the first dataset. The instructions, when executed by the at least one data processor, result in operations further comprising generating, based at least on a second dataset indicating a second epitope specificity of a second plurality of cells expressing a candidate antibody, a second reduced dimension representation of the second dataset. The instructions, when executed by the at least one data processor, result in operations further comprising determining, based at least on the first reduced dimension representation and the second reduced dimension representation, whether the second plurality of cells exhibit a novel epitope specificity or an epitope specificity of the epitope bin associated with the first plurality of cells.

In some embodiments, the first reduced dimension representation is generated by decomposing a first matrix comprising the first dataset, and wherein the second reduced dimension representation is generated by decomposing a second matrix comprising the second dataset. In some embodiments, the first matrix and/or the second matrix are decomposed by applying a principle component analysis (PCA), a neighborhood component analysis, a linear discriminant analysis, and/or a non-negative matrix factorization. In some embodiments, the first reduced dimension representation and the second reduced dimension representation is generated by embedding a reduced dimensional space. In some embodiments, the first reduced dimension representation and the second reduced dimension representation is generated by embedding a reduced dimensional space. In some embodiments, the reduced dimensional space is embedded by applying a t-distributed stochastic neighbor embedding (t-SNE), a uniform manifold approximation and projection (UMAP), and/or a generalized linear model principle component analysis (GLMPCA). In some embodiments, each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

In some embodiments, each element in the first matrix comprises a count of each of the first plurality of cells bound to the antigen, and wherein each element in the second matrix comprises a count of each of the second plurality of cells bound to the antigen. In some embodiments, the antigen is labeled with a reporter oligonucleotide. In some embodiments, the reporter oligonucleotide comprises at least one functional sequence, at least one common barcode, at least one capture sequence, and at least one UMI. In some embodiments, the antigen is selected from the group consisting of a protein, a viral-like particle, and a nanoparticle. In some embodiments, the antigen is a protein. In some embodiments, the antigen comprises a point mutation. In some embodiments, the point mutation alters the epitope specificity of the known antibody. In some embodiments, the first reduced dimension representation and the second reduced dimension representation are generated by applying a graph-based embedding and reduction technique. In some embodiments, at least one of the first dataset and the second dataset include one or more negative control antigens.

In some embodiments, the first reduced dimension representation includes a first cluster of cells corresponding to the first plurality of cells, and wherein the second reduced dimension representation includes a second cluster of cells corresponding to the second plurality of cells. In some embodiments, the second plurality of cells exhibit the novel epitope specificity or the epitope specificity associated with the first plurality of cells is determined based at least on a distribution of the second cluster of cells relative to the first cluster of cells.

In some embodiments, the instructions cause the at least one data processor to perform operations further comprises applying a clustering algorithm to validate the distribution of the second cluster of cells relative to the first cluster of cells. In some embodiments, the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity. In some embodiments, the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

In some embodiments, the instructions cause the at least one data processor to perform operations further comprising determining, for the first plurality of cells expressing the known antibody, a first plurality of scores representative of an epitope binding property of each of the first plurality of cells. The instructions cause the at least one data processor to perform operations further comprising determining, for the second plurality of cells expressing the candidate antibody, a second plurality of scores representative of the epitope binding property of each of the second plurality of cells. The instructions cause the at least one data processor to perform operations further comprising generating a first distribution of the first plurality of scores and a second distribution of the second plurality of scores. The instructions cause the at least one data processor to perform operations further comprising determining, based at least on a comparison between the first distribution and the second distribution, whether the second plurality of cells expressing the candidate antibody exhibits the novel epitope specificity or a same epitope specificity as one or more of the first plurality of cells.

In some embodiments, the comparison between the first distribution and the second distribution is performed by applying a Kolmogorov-Smirnov test and/or an outlier detection algorithm. In some embodiments, the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

In some embodiments, the first dataset and the second dataset are generated by at least engineering the first plurality of cells to express the known antibody and the second plurality of cells to express the candidate antibody; incubating the antigen with the first plurality of cells and the second plurality of cells, wherein the antigen is labeled with the reporter oligonucleotide; generating a plurality of single cell suspensions, wherein each of the plurality of single cell suspensions comprises one of either the first plurality of cells or the second plurality of cells bound to or not bound to the antigen; generating a library of barcoded nucleic acid molecules from the first plurality of cells and the second plurality of cells, wherein the library of barcoded nucleic acid molecules comprises the reporter sequence or complement thereof and sequences corresponding to immune receptors; and sequencing the library of barcoded nucleic acid molecules.

In some embodiments a computer-implemented method for determining an epitope specificity is provided. In some embodiments, the computer-implemented method comprises generating, based at least on a first dataset indicating a first epitope specificity of a first plurality of cells expressing a known antibody, a first reduced dimension representation of the first dataset. The computer-implemented method also comprises generating, based at least on a second dataset indicating a second epitope specificity of a second plurality of cells expressing a candidate antibody, a second reduced dimension representation of the second dataset. The computer-implemented method further comprises determining, based at least on the first reduced dimension representation and the second reduced dimension representation, whether the second plurality of cells exhibit a novel epitope specificity or an epitope specificity of the epitope bin associated with the first plurality of cells.

In some embodiments, the first reduced dimension representation is generated by decomposing a first matrix comprising the first dataset, and wherein the second reduced dimension representation is generated by decomposing a second matrix comprising the second dataset. In some embodiments, first matrix and/or the second matrix are decomposed by applying a principle component analysis (PCA), a neighborhood component analysis, a linear discriminant analysis, and/or a non-negative matrix factorization. In some embodiments, the first reduced dimension representation and the second reduced dimension representation is generated by embedding a reduced dimensional space. In some embodiments, the reduced dimensional space is embedded by applying a t-distributed stochastic neighbor embedding (t-SNE), a uniform manifold approximation and projection (UMAP), and/or a generalized linear model principle component analysis (GLMPCA). In some embodiments, each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

In some embodiments, each element in the first matrix comprises a count of each of the first plurality of cells bound to the antigen, and wherein each element in the second matrix comprises a count of each of the second plurality of cells bound to the antigen. In some embodiments, the antigen is labeled with a reporter oligonucleotide. In some embodiments, the reporter oligonucleotide comprises at least one functional sequence, at least one common barcode, at least one capture sequence, and at least one UMI. In some embodiments, the antigen is selected from the group consisting of a protein, a viral-like particle, and a nanoparticle. In some embodiments, the antigen is a protein. In some embodiments, the antigen comprises a point mutation. In some embodiments, the point mutation alters the epitope specificity of the known antibody. In some embodiments, the first reduced dimension representation and the second reduced dimension representation are generated by applying a graph-based embedding and reduction technique. In some embodiments, at least one of the first dataset and the second dataset include one or more negative control antigens.

In some embodiments, the first reduced dimension representation includes a first cluster of cells corresponding to the first plurality of cells, and wherein the second reduced dimension representation includes a second cluster of cells corresponding to the second plurality of cells. In some embodiments, whether the second plurality of cells exhibit the novel epitope specificity or the epitope specificity associated with the first plurality of cells is determined based at least on a distribution of the second cluster of cells relative to the first cluster of cells.

In some embodiments, the computer implemented method further comprises applying a clustering algorithm to validate the distribution of the second cluster of cells relative to the first cluster of cells. In some embodiments, the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity. In some embodiments, the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

In some embodiments, the computer implemented method further comprises determining, for the first plurality of cells expressing the known antibody, a first plurality of scores representative of an epitope binding property of each of the first plurality of cells; determining, for the second plurality of cells expressing the candidate antibody, a second plurality of scores representative of the epitope binding property of each of the second plurality of cells; generating a first distribution of the first plurality of scores and a second distribution of the second plurality of scores; and determining, based at least on a comparison between the first distribution and the second distribution, whether the second plurality of cells expressing the candidate antibody exhibits the novel epitope specificity or a same epitope specificity as one or more of the first plurality of cells.

In some embodiments, the comparison between the first distribution and the second distribution is performed by applying a Kolmogorov-Smirnov test and/or an outlier detection algorithm. In some embodiments, the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

In some embodiments, the first dataset and the second dataset are generated by at least engineering the first plurality of cells to express the known antibody and the second plurality of cells to express the candidate antibody; incubating the antigen with the first plurality of cells and the second plurality of cells, wherein the antigen is labeled with the reporter oligonucleotide; generating a plurality of single cell suspensions, wherein each of the plurality of single cell suspensions comprises one of either the first plurality of cells or the second plurality of cells bound to or not bound to the antigen; generating a library of barcoded nucleic acid molecules from the first plurality of cells and the second plurality of cells, wherein the library of barcoded nucleic acid molecules comprises the reporter sequence or complement thereof and sequences corresponding to immune receptors; and sequencing the library of barcoded nucleic acid molecules.

In some embodiments, a non-transitory computer readable medium for determining epitope specificity is provided. In some embodiments, the non-transitory computer readable medium storing instructions, which when executed by at least one data processor, result in operations comprising generating, based at least on a first dataset indicating a first epitope specificity of a first plurality of cells expressing a known antibody, a first reduced dimension representation of the first dataset; generating, based at least on a second dataset indicating a second epitope specificity of a second plurality of cells expressing a candidate antibody, a second reduced dimension representation of the second dataset; and determining, based at least on the first reduced dimension representation and the second reduced dimension representation, whether the second plurality of cells exhibit a novel epitope specificity or an epitope specificity of the epitope bin associated with the first plurality of cells.

In some embodiments, a method for determining epitope specificity is provided. The method comprising generating a plurality of single cell suspensions, wherein each of the plurality of single cell suspensions comprises one of either a first plurality of cells or a second plurality of cells bound to or not bound to the antigen; generating a library of barcoded nucleic acid molecules from the first plurality of cells and the second plurality of cells, wherein the library of barcoded nucleic acid molecules comprises a reporter sequence or complement thereof and sequences corresponding to immune receptors; sequencing the library of barcoded nucleic acid molecules; obtaining a first epitope specificity of the first plurality of cells and a second epitope specificity of the second plurality of cells by contacting the antigen with the first plurality of cells and the second plurality of cells, wherein the antigen is labeled with a reporter oligonucleotide comprising the reporter sequence, and wherein the first plurality of cells is engineered to express a known antibody and the second plurality of cells is engineered to express a candidate antibody; generating, a first reduced dimension representation of the first epitope specificity of the first plurality of cells expressing the known antibody; and a second reduced dimension representation of the second epitope specificity of the second plurality of cells expressing the candidate antibody; and determining, based at least on the first reduced dimension representation and the second reduced dimension representation, whether the second plurality of cells exhibit a novel epitope specificity or an epitope specificity associated with the first plurality of cells.

FIG. 13 depicts a block diagram illustrating an example of a computer system 1301, in accordance with some example embodiments. Referring to FIGS. 11 and 12A-D, the computer system 1301 may be configured to implement one or more of the analysis engine 1102, the sequencing platform 1104, and the client device 1106. The computer system 1301 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, the electronic storage unit 1315, interface 1320 and peripheral devices 1325 are in communication with the CPU 1305 through a communication bus (solid lines), such as a motherboard. The electronic storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The electronic storage unit 1315 can store files, such as drivers, libraries and saved programs. The electronic storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the CPU 1305. In some cases, the code can be retrieved from the electronic storage unit 1315 and stored on the memory 1310 for ready access by the CPU 1305. In some situations, the electronic storage unit 1315 can be precluded, and machine-executable instructions are stored on memory 1310.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that includes a user interface (UI) 1340 for providing, for example, results of the assay, such as a summary of one or more antigen binding molecules that bind the antigens, a summary of one or more antigens not bound by an antigen binding molecule in the composition, a site on the antigen that binds to the antigen binding molecule, or proposed modifications to the antigen that can reduce affinity of the antigen binding fragment for the antigen. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, contact an antigen with an antigen binding molecule, isolate the antigen binding molecule, or identify the antigen binding molecule as described herein. In some embodiments, an algorithm can determine a site on the antigen binding to the antigen binding molecule, such as an epitope. In some embodiments, an algorithm can determine a modification of an antigen or to modify an antigen to reduce affinity of the antigen binding molecule for the antigen.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

What is claimed is:

1. A method, comprising:
(a) obtaining a first epitope specificity of a first plurality of cells and a second epitope specificity of a second plurality of cells by contacting an antigen with the first plurality of cells and the second plurality of cells, wherein the antigen is labeled with a reporter oligonucleotide comprising a reporter sequence, and wherein the first plurality of cells is engineered to express a known antibody and the second plurality of cells is engineered to express a candidate antibody;
(b) generating,
i). a first reduced dimension representation of the first epitope specificity of the first plurality of cells expressing the known antibody; and
ii). a second reduced dimension representation of the second epitope specificity of the second plurality of cells expressing the candidate antibody;
(c) determining, based at least on the first reduced dimension representation and the second reduced dimension representation, whether the second plurality of cells exhibit a novel epitope specificity or an epitope specificity associated with the first plurality of cells.

2. A method, comprising:
(a) obtaining a first epitope specificity of a first plurality of cells and a second epitope specificity of a second plurality of cells by contacting an antigen with the first plurality of cells and the second plurality of cells, wherein the antigen is labeled with a reporter oligonucleotide comprising a reporter sequence, and wherein the first plurality of cells is engineered to express a known antibody and the second plurality of cells is engineered to express a candidate antibody; and
(b) applying, by at least one data processor, one or more data analysis techniques to determine whether the second plurality of cells exhibit a novel epitope specificity or an epitope specificity associated with the first plurality of cells.

3. The method of claim 1 or 2, wherein the obtaining the first epitope specificity and the second epitope specificity further comprises:
i). generating a plurality of single cell suspensions, wherein each of the plurality of single cell suspensions comprises one of either the first plurality of cells or the second plurality of cells bound to or not bound to the antigen;
ii). generating a library of barcoded nucleic acid molecules from the first plurality of cells and the second plurality of cells, wherein the library of barcoded nucleic acid molecules comprises the reporter sequence or complement thereof and sequences corresponding to immune receptors; and
iii). sequencing the library of barcoded nucleic acid molecules.

4. The method of claim 3, wherein (i) comprises:
following the contacting the antigen with the first plurality of cells and the second plurality of cells, partitioning the first plurality of cells and the second plurality of cells into a plurality of partitions, wherein a partition of the plurality of partitions comprises a cell of the first plurality of cells or the second plurality of cells bound to the antigen, and a plurality of nucleic acid barcode molecules wherein a first nucleic acid barcode molecule of the plurality of nucleic acid barcode molecules comprises a partition barcode sequence.

5. The method of claim 3, wherein (ii) comprises:
a. in the partition, coupling the first nucleic acid barcode molecule to the reporter oligonucleotide, and
b. using the reporter oligonucleotide coupled to the first nucleic acid barcode molecule to generate a first barcoded nucleic acid molecule comprising the reporter sequence or a reverse complement thereof and the partition barcode sequence or a reverse complement thereof

6. The method of claim 3, wherein a second nucleic acid barcode molecule of the plurality of nucleic acid barcode molecules comprises the partition barcode sequence, and wherein (ii) further comprises:
a. in the partition, coupling the second nucleic acid barcode molecule to a nucleic acid analyte of the cell bound to the antigen, the nucleic acid analyte comprising a sequence of an immune receptor expressed by the cell bound to the antigen, or a reverse complement thereof, and
b. using the nucleic acid analyte of the cell bound to the antigen coupled to the second nucleic acid barcode molecule to generate a second barcoded nucleic acid molecule comprising the sequence of the immune receptor expressed by the cell bound to the antigen, or a reverse complement thereof.

7. The method of claim 3, wherein the plurality of nucleic acid barcode molecules is attached to a bead, and wherein the partition barcode sequence identifies the bead.

8. The method of claim 3, wherein the first nucleic acid barcode molecule comprises a first capture sequence configured to couple to the reporter oligonucleotide.

9. The method of claim 8, wherein the reporter oligonucleotide further comprises a capture handle sequence complementary to the first capture sequence.

10. The method of claim 3, wherein the second nucleic acid barcode molecule further comprises a second capture sequence configured to couple to the nucleic acid analyte of the cell bound to the antigen.

11. The method of claim 10, wherein the nucleic acid analyte is an mRNA analyte or a cDNA molecule generated from the mRNA analyte.

12. The method of claim 11, wherein the second capture sequence is a template switch oligonucleotide sequence.

13. The method of claim 3, wherein the first capture sequence and the second capture sequence are identical.

14. The method of claim 3, wherein the first capture sequence and the second capture sequence are different.

15. The method of claim 3, wherein the partition is a droplet.

16. The method of claim 3, wherein the partition is a well.

17. The method of claim 3, further comprising:
contacting the first plurality of cells with a first cell group labeling agent comprising a first cell group reporter oligonucleotide, the first cell group reporter oligonucleotide comprising a first cell group reporter sequence that identifies the first plurality of cells, and
contacting the second plurality of cells with a second cell group labeling agent, the second cell group labeling agent comprising a second cell group reporter oligonucleotide comprising a second cell group reporter sequence that identifies the second plurality of cells.

18. The method of claim 17, wherein a third nucleic acid barcode molecule of the plurality of nucleic acid barcode molecules comprises the partition barcode sequence, and wherein (ii) comprises
a. in the partition, coupling the third nucleic acid barcode molecule to the first cell group reporter oligonucleotide or to the second cell group reporter oligonucleotide, and
b. using the third nucleic acid barcode molecule coupled to the first cell group reporter oligonucleotide or to the second cell group reporter oligonucleotide to generate a third barcoded nucleic acid molecule comprising the first cell group reporter sequence or the second cell group reporter sequence, or a reverse complement thereof, and the partition barcode sequence or a reverse complement thereof.

19. The method of claim 3, further comprising determining a sequence of the first barcoded nucleic acid molecule or a derivative thereof, the second barcoded nucleic acid molecule or a derivative thereof, and/or the third barcoded nucleic acid molecule or a derivative thereof.

20. The method of claim 1 or 2, wherein the first reduced dimension representation is generated by decomposing a first matrix including a first dataset indicating the first epitope specificity of the first plurality of cells, and wherein the second reduced dimension representation is generated by decomposing a second matrix including a second dataset indicating the second epitope specificity of the second plurality of cells.

21. The method of claim 20, wherein the first matrix and/or the second matrix are decomposed by applying a principle component analysis (PCA), a neighborhood component analysis, a linear discriminant analysis, and/or a non-negative matrix factorization.

22. The method of any one of claims 20-21, wherein each of the first matrix and the second matrix includes a row corresponding to the antigen, wherein each column in the first matrix corresponds to one of the first plurality of cells expressing the known antibody, and wherein each column in the second matrix corresponds to one of the second plurality of cells expressing the candidate antibody.

23. The method of any one of claims 21-22, wherein each element in the first matrix comprises a count of each of the first plurality of cells bound to the antigen, and wherein each element in the second matrix comprises a count of each of the second plurality of cells bound to the antigen.

24. The method of any one of claims 1-23, wherein the first reduced dimension representation and the second reduced dimension representation are generated by embedding a reduced dimensional space.

25. The method of claim 24, wherein the reduced dimensional space is embedded by applying a t-distributed stochastic neighbor embedding (t-SNE), a uniform manifold approximation and projection (UMAP), and/or a generalized linear model principle component analysis (GLMPCA).

26. The method of any one of claims 1-25, wherein the first reduced dimension representation and the second reduced dimension representation are generated by applying a graph-based embedding and reduction technique.

27. The method of any one of claims 1-26, the reporter oligonucleotide comprises an additional functional sequence.

28. The method of claim 27, wherein said additional functional sequence is selected from at least one functional sequence, at least one common barcode, at least one capture sequence, and at least one UMI.

29. The method of any of claims 1-28, wherein the antigen is selected from the group consisting of a protein, a viral-like particle, and a nanoparticle.

30. The method of any of claims 1-29, wherein the antigen is a protein.

31. The method of any of claims 1-30, wherein the antigen comprises a point mutation.

32. The method of any of claims 30-31, wherein the point mutation alters the epitope specificity of the known antibody.

33. The method of any one of claims 1-32, wherein the first reduced dimension representation includes a first cluster of cells corresponding to the first plurality of cells, and wherein the second reduced dimension representation includes a second cluster of cells corresponding to the second plurality of cells.

34. The method of claim 33, wherein whether the second plurality of cells exhibit the novel epitope specificity or the epitope specificity associated with the first plurality of cells is determined based at least on a distribution of the second cluster of cells relative to the first cluster of cells.

35. The method of claim 34, further comprising applying a clustering algorithm to validate the distribution of the second cluster of cells relative to the first cluster of cells.

36. The method of any one of claims 33-35, wherein the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

37. The method of any one of claims 33-36, wherein the second plurality of cells are determined to exhibit the epitope specificity of the first plurality of cells based at least on the second cluster of cells being within a threshold distance of the first cluster of cells.

38. The method of any one of claims 33-37, wherein the second plurality of cells are determined to exhibit the novel epitope specificity based at least on the second cluster of cells being more than a threshold distance away from the first cluster of cells.

39. The method of any one of claims 1-38, wherein at least one of the first dataset and the second dataset include one or more negative control antigens.

40. The method of any one of claims 1-39, further comprising:
determining, for the first plurality of cells expressing the known antibody, a first plurality of scores representative of an epitope binding property of each of the first plurality of cells;
determining, for the second plurality of cells expressing the candidate antibody, a second plurality of scores representative of the epitope binding property of each of the second plurality of cells;
generating a first distribution of the first plurality of scores and a second distribution of the second plurality of scores; and
determining, based at least on a comparison between the first distribution and the second distribution, whether the second plurality of cells expressing the candidate antibody exhibits the novel epitope specificity or a same epitope specificity as one or more of the first plurality of cells.

41. The method of claim 40, wherein the comparison between the first distribution and the second distribution is performed by applying a Kolmogorov-Smirnov test and/or an outlier detection algorithm.

42. The method of any one of claims 40-41, wherein the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

43. A system comprising:
at least one data processor; and
at least one memory storing instructions, which when executed by the at least one data processor, result in operations comprising:
generating, based at least on a first dataset indicating a first epitope specificity of a first plurality of cells expressing a known antibody, a first reduced dimension representation of the first dataset;
generating, based at least on a second dataset indicating a second epitope specificity of a second plurality of cells expressing a candidate antibody, a second reduced dimension representation of the second dataset; and
determining, based at least on the first reduced dimension representation and the second reduced dimension representation, whether the second plurality of cells exhibit a novel epitope specificity or an epitope specificity of the epitope bin associated with the first plurality of cells.

44. The system of claim 43, wherein the first reduced dimension representation is generated by decomposing a first matrix comprising the first dataset, and wherein the second reduced dimension representation is generated by decomposing a second matrix comprising the second dataset.

45. The system of claim 44, wherein the first matrix and/or the second matrix are decomposed by applying a principle component analysis (PCA), a neighborhood component analysis, a linear discriminant analysis, and/or a non-negative matrix factorization.

46. The system of any of one of claims 43-45, wherein the first reduced dimension representation and the second reduced dimension representation is generated by embedding a reduced dimensional space.

47. The system of claim 46, wherein the reduced dimensional space is embedded by applying a t-distributed stochastic neighbor embedding (t-SNE), a uniform manifold approximation and projection (UMAP), and/or a generalized linear model principle component analysis (GLMPCA).

48. The system of any of one of claims 44-47, wherein each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

49. The system of claim 48, wherein each element in the first matrix comprises a count of each of the first plurality of cells bound to the antigen, and wherein each element in the second matrix comprises a count of each of the second plurality of cells bound to the antigen.

50. The system of any one of claims 48-49, wherein the antigen is labeled with a reporter oligonucleotide.

51. The system of claim 50, the reporter oligonucleotide comprises at least one functional sequence, at least one common barcode, at least one capture sequence, and at least one UMI.

52. The system of any one of claims 48-51, wherein the antigen is selected from the group consisting of a protein, a viral-like particle, and a nanoparticle.

53. The system of any one of claims 48-52, wherein the antigen is a protein.

54. The system of any one of claims 48-53, wherein the antigen comprises a point mutation.

55. The system of claim 54, wherein the point mutation alters the epitope specificity of the known antibody.

56. The system of any one of claims 43-55, wherein the first reduced dimension representation and the second reduced dimension representation are generated by applying a graph-based embedding and reduction technique.

57. The system of any one of claims 43-56, wherein at least one of the first dataset and the second dataset include one or more negative control antigens.

58. The system of any one of claims 43-57, wherein the first reduced dimension representation includes a first cluster of cells corresponding to the first plurality of cells, and wherein the second reduced dimension representation includes a second cluster of cells corresponding to the second plurality of cells.

59. The system of claim 58, wherein whether the second plurality of cells exhibit the novel epitope specificity or the epitope specificity associated with the first plurality of cells is determined based at least on a distribution of the second cluster of cells relative to the first cluster of cells.

60. The system of claim 59, further comprising applying a clustering algorithm to validate the distribution of the second cluster of cells relative to the first cluster of cells.

61. The system of any one of claims 58-60, wherein the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

62. The system of any one of claims 58-61, wherein the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

63. The system of any one of claims 43-62, further comprising:
determining, for the first plurality of cells expressing the known antibody, a first plurality of scores representative of an epitope binding property of each of the first plurality of cells;
determining, for the second plurality of cells expressing the candidate antibody, a second plurality of scores representative of the epitope binding property of each of the second plurality of cells;
generating a first distribution of the first plurality of scores and a second distribution of the second plurality of scores; and
determining, based at least on a comparison between the first distribution and the second distribution, whether the second plurality of cells expressing the candidate antibody exhibits the novel epitope specificity or a same epitope specificity as one or more of the first plurality of cells.

64. The system of claim 63, wherein the comparison between the first distribution and the second distribution is performed by applying a Kolmogorov-Smirnov test and/or an outlier detection algorithm.

65. The system of claim 63 or 64, wherein the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

66. The system of any one of claims 43-65, wherein the first dataset and the second dataset are generated by at least:
(a) engineering the first plurality of cells to express the known antibody and the second plurality of cells to express the candidate antibody;
(b) incubating the antigen with the first plurality of cells and the second plurality of cells, wherein the antigen is labeled with the reporter oligonucleotide;
(c) generating a plurality of single cell suspensions, wherein each of the plurality of single cell suspensions comprises one of either the first plurality of cells or the second plurality of cells bound to or not bound to the antigen;
(d) generating a library of barcoded nucleic acid molecules from the first plurality of cells and the second plurality of cells, wherein the library of barcoded nucleic acid molecules comprises the reporter sequence or complement thereof and sequences corresponding to immune receptors; and
(e) sequencing the library of barcoded nucleic acid molecules.

67. An antibody identified by the system of any one of claims 43-66, wherein the antibody is identified based at least on the second cluster of cells exhibiting the novel epitope specificity or the epitope specificity of the epitope bin associated with the first cluster of cells.

68. The antibody of claim 67, wherein the antibody is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody.

69. The antibody of claim 67 or 68, wherein the antibody is an oligoclonal antibody.

70. A computer-implemented method, comprising:
generating, based at least on a first dataset indicating a first epitope specificity of a first plurality of cells expressing a known antibody, a first reduced dimension representation of the first dataset;
generating, based at least on a second dataset indicating a second epitope specificity of a second plurality of cells expressing a candidate antibody, a second reduced dimension representation of the second dataset; and
determining, based at least on the first reduced dimension representation and the second reduced dimension representation, whether the second plurality of cells exhibit a novel epitope specificity or an epitope specificity of the epitope bin associated with the first plurality of cells.

71. The computer implemented method of claim 70, wherein the first reduced dimension representation is generated by decomposing a first matrix comprising the first dataset, and wherein the second reduced dimension representation is generated by decomposing a second matrix comprising the second dataset.

72. The computer implemented method of claim 71, wherein the first matrix and/or the second matrix are decomposed by applying a principle component analysis (PCA), a neighborhood component analysis, a linear discriminant analysis, and/or a non-negative matrix factorization.

73. The computer implemented method of any of one of claims 70-72, wherein the first reduced dimension representation and the second reduced dimension representation is generated by embedding a reduced dimensional space.

74. The computer implemented method of claim 73, wherein the reduced dimensional space is embedded by applying a t-distributed stochastic neighbor embedding (t-SNE), a uniform manifold approximation and projection (UMAP), and/or a generalized linear model principle component analysis (GLMPCA).

75. The computer implemented method of any of one of claims 71-74, wherein each row of the first matrix and the second matrix corresponds to an antigen, wherein each column in the first matrix corresponds to one of the first plurality of cells expressing the known antibody, and wherein each column in the second matrix corresponds to one of the second plurality of cells expressing the candidate antibody.

76. The computer implemented method of claim 75, wherein each element in the first matrix comprises a count of each of the first plurality of cells bound to the antigen, and wherein each element in the second matrix comprises a count of each of the second plurality of cells bound to the antigen.

77. The computer implemented method of any one of claims 75-76, wherein the antigen is labeled with a reporter oligonucleotide.

78. The computer implemented method of claim 77, the reporter oligonucleotide comprises at least one functional sequence, at least one common barcode, at least one capture sequence, and at least one UMI.

79. The computer implemented method of any one of claims 75-78, wherein the antigen is selected from the group consisting of a protein, a viral-like particle, and a nanoparticle.

80. The computer implemented method of any one of claims 75-79, wherein the antigen is a protein.

81. The computer implemented method of any one of claims 75-80, wherein the antigen comprises a point mutation.

82. The computer implemented method of claim 81, wherein the point mutation alters the epitope specificity of the known antibody.

83. The computer implemented method of any one of claims 70-82, wherein the first reduced dimension representation and the second reduced dimension representation are generated by applying a graph-based embedding and reduction technique.

84. The computer implemented method of any one of claims 70-83 wherein at least one of the first dataset and the second dataset include one or more negative control antigens.

85. The computer implemented method of any one of claims 70-84, wherein the first reduced dimension representation includes a first cluster of cells corresponding to the first plurality of cells, and wherein the second reduced dimension representation includes a second cluster of cells corresponding to the second plurality of cells.

86. The computer implemented method of claim 85, wherein whether the second plurality of cells exhibit the novel epitope specificity or the epitope specificity associated with the first plurality of cells is determined based at least on a distribution of the second cluster of cells relative to the first cluster of cells.

87. The computer implemented method of claim 86, further comprising applying a clustering algorithm to validate the distribution of the second cluster of cells relative to the first cluster of cells.

88. The computer implemented method of any one of claims 85-87, wherein the first cluster of cells and the second cluster of cells form a same epitope bin when the second plurality of cells are determined to exhibit the epitope specificity of the first plurality of cells, and wherein the first cluster of cells and the second cluster of cells form different epitope bins when the second plurality of cells are determined to exhibit the novel epitope specificity.

89. The computer implemented method of any one of claims 85-88, wherein the second plurality of cells are determined to exhibit the epitope specificity of the first plurality of cells based at least on the second cluster of cells being within a threshold distance of the first cluster of cells, and wherein the second plurality of cells are determined to exhibit the novel epitope specificity based at least on the second cluster of cells being more than the threshold distance away from the first cluster of cells.

90. The computer implemented method of any one of claims 70-89, further comprising:
determining, for the first plurality of cells expressing the known antibody, a first plurality of scores representative of an epitope binding property of each of the first plurality of cells;
determining, for the second plurality of cells expressing the candidate antibody, a second plurality of scores representative of the epitope binding property of each of the second plurality of cells;
generating a first distribution of the first plurality of scores and a second distribution of the second plurality of scores; and
determining, based at least on a comparison between the first distribution and the second distribution, whether the second plurality of cells expressing the candidate antibody exhibits the novel epitope specificity or a same epitope specificity as one or more of the first plurality of cells.

91. The computer implemented method of claim 90, wherein the comparison between the first distribution and the second distribution is performed by applying a Kolmogorov-Smirnov test and/or an outlier detection algorithm.

92. The computer implemented method of any of claims 90-91, wherein the first plurality of scores and the second plurality of scores are generated by applying a centered log ratio transformation to a plurality of vectors, and wherein each of the plurality of vectors include one or more counts corresponding to the epitope binding property of the first plurality of cells or the second plurality of cells.

93. The computer implemented method of any one of claims 70-92, wherein the first dataset and the second dataset are generated by at least:
(f) engineering the first plurality of cells to express the known antibody and the second plurality of cells to express the candidate antibody;
(g) incubating the antigen with the first plurality of cells and the second plurality of cells, wherein the antigen is labeled with the reporter oligonucleotide;
(h) generating a plurality of single cell suspensions, wherein each of the plurality of single cell suspensions comprises one of either the first plurality of cells or the second plurality of cells bound to or not bound to the antigen; and
(i) generating a library of barcoded nucleic acid molecules from the first plurality of cells and the second plurality of cells, wherein the library of barcoded nucleic acid molecules comprises the reporter sequence or complement thereof and sequences corresponding to immune receptors; and
(j) sequencing the library of barcoded nucleic acid molecules.

94. An antibody identified by the system of any one of claims 70-93, wherein the antibody is identified based at least on the second cluster of cells exhibiting the novel epitope specificity or the epitope specificity of the epitope bin associated with the first cluster of cells.

95. The antibody of claim 94, wherein the antibody is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody.

96. The antibody of any of claims 94-95, wherein the antibody is an oligoclonal antibody.

97. A non-transitory computer readable medium storing instructions, which when executed by at least one data processor, result in operations comprising:
generating, based at least on a first dataset indicating a first epitope specificity of a first plurality of cells expressing a known antibody, a first reduced dimension representation of the first dataset;
generating, based at least on a second dataset indicating a second epitope specificity of a second plurality of cells expressing a candidate antibody, a second reduced dimension representation of the second dataset; and
determining, based at least on the first reduced dimension representation and the second reduced dimension representation, whether the second plurality of cells exhibit a novel epitope specificity or an epitope specificity of the epitope bin associated with the first plurality of cells.

98. A method, comprising:
(a) generating a plurality of single cell suspensions, wherein each of the plurality of single cell suspensions comprises one of either a first plurality of cells or a second plurality of cells bound to or not bound to the antigen;
(b) generating a library of barcoded nucleic acid molecules from the first plurality of cells and the second plurality of cells, wherein the library of barcoded nucleic acid molecules comprises a reporter sequence or complement thereof and sequences corresponding to immune receptors;
(c) sequencing the library of barcoded nucleic acid molecules;
(d) obtaining a first epitope specificity of the first plurality of cells and a second epitope specificity of the second plurality of cells by contacting the antigen with the first plurality of cells and the second plurality of cells, wherein the antigen is labeled with a reporter oligonucleotide comprising the reporter sequence, and wherein the first plurality of cells is engineered to express a known antibody and the second plurality of cells is engineered to express a candidate antibody;
(e) generating,
i). a first reduced dimension representation of the first epitope specificity of the first plurality of cells expressing the known antibody; and
ii). a second reduced dimension representation of the second epitope specificity of the second plurality of cells expressing the candidate antibody; and
(f) determining, based at least on the first reduced dimension representation and the second reduced dimension representation, whether the second plurality of cells exhibit a novel epitope specificity or an epitope specificity associated with the first plurality of cells.
